(19) United States
(12) Patent Application Publication
(10) Pub. No.: US 2014/0137284 A1
(43) Pub. Date: May 15, 2014

(54) Method of Producing Plants Having Increased Resistance to Pathogens

(71) Applicant: BASF Plant Science Company GmbH, Ludwigshafen (DE)

(72) Inventors: Holger SCHULTHEISS, Boehl-Iggelheim (DE); Tobias MENTZEL, Roemerberg (DE); Patrick SCHWEIZER, Ballenstedt (DE); Dimitar DOUCHKOV, Hedersleben (DE); Axel HIMMELBACH, Thale (DE)

(73) Assignee: BASF Plant Science Company GmbH, Ludwigshafen (DE)

(21) Appl. No.: 14/074,143

(22) Filed: Nov. 7, 2013

Related U.S. Application Data
(60) Provisional application No. 61/726,601, filed on Nov. 15, 2012.

(51) Int. Cl. C12N 15/82

(52) U.S. Cl. CPC C12N 15/8282
USPC 800/265; 435/468; 435/419; 435/320.1; 800/276; 800/279; 800/301; 426/622

(57) ABSTRACT

The present invention relates to a method of producing a transgenic plant cell, a transgenic plant or a transgenic part thereof having an increased resistance to pathogens, wherein the content and/or activity of a receptor-like protein kinase is increased.

### FIELD OF THE INVENTION

The present invention relates to a method of producing a transgenic plant cell, a transgenic plant or a transgenic part thereof having an increased resistance to pathogens, wherein the content and/or activity of a receptor-like protein kinase is increased.

### BACKGROUND OF THE INVENTION

Plant diseases, which are caused by various pathogens such as viruses, bacteria and fungi, may lead to significant crop losses of cultivated plants, resulting in economic consequences and in threatening human food supply. For example, infestation of cereals with Blumeria graminis, the pathogen that causes powdery mildew, may cause yield losses of up to 30%.

Since the last century, chemical fungicides have been utilised for controlling fungal diseases. A different approach is to examine the natural pathogen defence of plants against different pathogens and to use the same specifically for the production of pathogen resistant plants by gene technological manipulation, e.g. by means of introducing external resistance genes or by means of manipulating the endogenous gene expression of the plants.

Resistance is the ability of a plant to inhibit or at least limit any infestation or population of a pest. The plants have a certain degree of natural resistance which is imparted by the formation of specific defence substances, such as isoprenoids, flavonoids, enzymes and reactive oxygen species.

Therefore, one approach for producing pathogen resistant plants is the (over)expression of a transgene in said plants, resulting in the formation of specific defence substances. For example, chitinase (WO 92/17591) and pathogenesis-related genes (WO 92/20800) as well as genes for various oxidizing enzymes, such as glucose oxidase (WO 95/21924) and oxalate oxidase (WO 99/04013), have already been overexpressed in plants, thus creating plants having increased fungal resistance.

Conversely, it could be shown that some of the plant genes help a fungus to enter the plant. Thus, an alternative approach for producing transgenic plants having increased fungal resistance is to inhibit the expression of said plant genes which code for example for a polyphenoloxidase (WO 02/061101), NADPH oxidase (WO 2004/009820) and the Mlo gene (WO 00/01722) in transgenic plants.

Another alternative for causing resistance to pathogenic fungi is to introduce gene constructs into plants which inhibit the expression and/or activity of fungal genes that are essential for the proliferation and/or development of fungi (US 2007/0061918).

One type of resistance is the nonhost resistance which is usually defined as the durable resistance of all known genotypes of a plant species to all known races or isolates of a pathogen species. However, it may also operate at the subspecies level, for example with respect to formae speciales of Blumeria graminis. Hence, barley (Hordeum vulgare) shows resistance to B. graminis fsp. tritici, but is susceptible to B. graminis fsp. hordei. Conversely, wheat (Triticum aestivum) shows resistance to B. graminis fsp. hordei, but is susceptible to B. graminis fsp. tritici. Hence, it would be desirable to provide methods with which resistance can be transferred from plant species which are resistant to a specific pathogen to those which are susceptible to said pathogen.

Further, there is still a need to identify further genes which code for polypeptides involved in pathogen resistance and to develop methods for producing transgenic plants with increased pathogen resistance by using these genes.

### OBJECT AND SUMMARY OF THE INVENTION

It is thus an object of the present invention to identify genes which are involved in the pathogen resistance of plants.

It is a further object of the present invention to provide a method for producing transgenic plants with increased pathogen resistance, preferably resistance to fungal pathogens such as Blumeria graminis, Septoria tritici and/or Puccinia triticina.

These and further objects of the invention, as will become apparent from the description, are attained by the subject-matter of the independent claims.

Some of the preferred embodiments of the present invention form the subject-matter of the dependent claims.

The present inventors have found that the transgenic expression of a receptor-like kinase leads to an enhanced resistance of wheat cells to Blumeria graminis f.sp. tritici.

Receptor-like protein kinases (RLKs) are a large group of kinases with an extracellular domain, a single transmembrane domain and a cytoplasmic kinase domain. Due to this structure, they resemble the receptor tyrosine kinases in animals. In Arabidopsis more than 600 RLKs have been identified (Shiu and Bleecker (2001) Proc. Natl. Acad. Sci. USA 98(19): 10763-10768). They transduce extracellular signals into the cell and are thus involved in cellular signaling pathways regulating plant development, disease resistance and self-incompatibility (Baudino et al. (2001) Planta 213: 1-10).

In barley, the receptor-like protein kinase HvLysMR1 is induced during leaf senescence and heavy metal stress (Ouelhadj et al. (2007) J. Exp. Bot. 58(6): 1381-1396). Other receptor-like proteins from barley have been associated with pathogen resistance (Nirmala et al. (2007) Proc. Natl. Acad. Sci. USA 104(24): 10276-10281; Rayapuram et al. (2012) Mol. Plant Pathol. 13(2): 135-147). Further, in wheat it has been demonstrated that knocking down the receptor-like kinases TaRLK-R1, 2 or 3 compromises the hypersensitive reaction of wheat to stripe rust fungus (Zhou et al. (2007) Plant J. 52(3): 420-434).

The present invention provides a method of producing a transgenic plant cell, a transgenic plant or a transgenic part thereof having an increased resistance to pathogens compared to a control plant cell, plant or plant part, wherein in the transgenic plant cell, the transgenic plant or the transgenic part thereof the content and/or activity of a receptor-like protein kinase which is encoded by a nucleic acid sequence selected from the group consisting of:
a) a nucleic acid sequence comprising the sequence according to any of SEQ ID Nos. 1-5, 7-9, 11-13, 15-17, 19-21, 23 and 24 or a fragment of any of these sequences;
b) a nucleic acid sequence encoding a protein comprising the amino acid sequence according to any of SEQ ID Nos. 6, 10, 14, 18, 22 and 25;
c) a nucleic acid sequence comprising a sequence which is at least 70% identical to the sequence according to any of SEQ ID Nos. 1-5, 7-9, 11-13, 15-17, 19-21, 23 and 24 or a fragment of any of these sequences; and
d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences
   is increased in comparison to the control plant cell, plant or plant part.

The present invention also provides a method for increasing pathogen resistance in a transgenic plant cell, a transgenic plant or a transgenic part thereof compared to a control plant cell, plant or plant part, wherein in the transgenic plant cell, the transgenic plant or the transgenic part thereof the content and/or activity of a receptor-like protein kinase which is encoded by a nucleic acid sequence selected from the group consisting of:
a) a nucleic acid sequence comprising the sequence according to any of SEQ ID Nos. 1-5, 7-9, 11-13, 15-17, 19-21, 23 and 24 or a fragment of any of these sequences;
b) a nucleic acid sequence encoding a protein comprising the amino acid sequence according to any of SEQ ID Nos. 6, 10, 14, 18, 22 and 25;
c) a nucleic acid sequence comprising a sequence which is at least 70% identical to the sequence according to any of SEQ ID Nos. 1-5, 7-9, 11-13, 15-17, 19-21, 23 and 24 or a fragment of any of these sequences; and
d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences
   is increased in comparison to the control plant cell, plant or plant part.

Preferably, the method comprises the steps of
(a) introducing into a plant cell a vector which comprises:
   (i) a promoter functional in plant cells,
   (ii) operatively linked thereto at least one nucleic acid sequence as defined above;
   (iii) optionally, a termination sequence; to produce a transformed plant cell; and
(b) optionally, regenerating a transgenic plant from the transformed cell.

In a preferred embodiment the method further comprises step (b) or, if step (b) is present in the above method, step (c) of selecting transgenic plant cells or transgenic plants.

Preferably, the promoter is a tissue-specific and/or a pathogen-inducible promoter.

In another preferred embodiment, the method further comprises reducing the content and/or activity of at least one protein which mediates pathogen susceptibility or increasing the content and/or activity of at least one further protein which mediates pathogen resistance.

In another embodiment the method further comprises the step of crossing the transgenic plant produced by the above method with another plant in which the content and/or the activity of the receptor-like protein kinase as defined herein is not increased and selecting transgenic progeny in which the content and/or the activity of the receptor-like protein kinase as defined herein is increased.

In a preferred embodiment the method is for producing true breeding plants and comprises inbreeding the transgenic progeny of the above crossing and repeating this inbreeding step until a true breeding plant is obtained.

Another embodiment of the present invention relates to a method of producing or obtaining mutant plants, plant cells or plant parts having an increased resistance to pathogens compared to control plants, plant cells or plant parts, comprising the steps of:
(a) mutagenizing plant material;
(b) identifying plant material having at least one point mutation in an endogenous nucleic acid sequence having at least 70%, at least 80%, at least 90%, at least 95% or even 100% sequence identity to the nucleic acid sequence according to any of SEQ ID Nos. 1-5, 7-9, 11-13, 15-17, 19-21, 23 and 24 compared to the corresponding endogenous nucleic acid sequence in the control plant.

In a preferred embodiment, the method for producing or obtaining mutant plants, plant cells, or plant parts having an increased resistance to pathogens compared to control plants, plant cells, or plant parts, respectively, further comprises step (c) of obtaining a plant, plant cell or plant part from said plant material having at least one point mutation in the endogenous nucleic acid sequence having at least 70%, at least 80%, at least 90%, at least 95% or even 100% sequence identity to the nucleic acid sequence according to any of SEQ ID Nos. 1-5, 7-9, 11-13, 15-17, 19-21, 23 and 24 and/or the step of (d) selecting a plant, plant cell or plant part which has an increased resistance to pathogens compared to control plants, plant cells or plant parts.

In a further preferred embodiment the transgenic or mutant plant is a monocotyledonous plant, more preferably it is a barley or a wheat plant, most preferably it is a wheat plant.

Preferably, the transgenic or mutant plant has an increased resistance to a fungal pathogen, more preferably to Blumeria graminis, Septoria tritici and/or Puccinia triticina.

Even more preferably, the transgenic or mutant plant is a wheat plant and the pathogen is Blumeria graminis fsp. tritici. Hence, the resistance conferred by the present invention is non-host resistance.

In another embodiment the present invention relates to an expression construct comprising at least one nucleic acid sequence selected from the group consisting of:
a) a nucleic acid sequence comprising the sequence according to any of SEQ ID Nos. 1-5, 7-9, 11-13, 15-17, 19-21, 23 and 24 or a fragment of any of these sequences;
b) a nucleic acid sequence encoding a protein comprising the amino acid sequence according to any of SEQ ID Nos. 6, 10, 14, 18, 22 and 25;
c) a nucleic acid sequence comprising a sequence which is at least 70% identical to the sequence according to any of SEQ ID Nos. 1-5, 7-9, 11-13, 15-17, 19-21, 23 and 24 or a fragment of any of these sequences; and
d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences.
   operatively linked to a promoter functional in plant cells.

In a preferred embodiment the expression construct further comprises regulatory sequences which can act as termination and/or polyadenylation signal in the plant cell and which are operably linked to the DNA sequence as defined herein.

In another preferred embodiment the promoter is a tissue-specific and/or a pathogen-inducible promoter.

In another embodiment the invention relates to a vector comprising the expression construct as defined above.

A preferred embodiment is the use of an expression construct or vector as described herein for the transformation of a plant, plant part, or plant cell to provide a pathogen resistant plant, plant part, or plant cell. Thus, a preferred embodiment is the use of an expression construct or a vector as described herein for increasing pathogen resistance in a plant, plant part, or plant cell compared to a control plant, plant part, or plant cell.

In still a further embodiment the invention relates to a transgenic or mutant plant, plant cell or plant part with an increased resistance to pathogens compared to a control plant, plant cell or plant part, which plant is produced by the method of the present invention or contains an expression construct or a vector of the present invention.

In another embodiment the invention relates to the use of the transgenic or mutant plant or parts thereof as fodder material or to produce feed material.

The present invention also relates to transgenic or mutant seed produced from the transgenic or mutant plant and to flour produced from said transgenic or mutant seed, wherein the presence of the transgene, the expression construct, the vector or the mutation which increases the content and/or the activity of the receptor-like protein kinase as defined herein can be detected in said transgenic or mutant seed or in said flour.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a plant is defined to be obtainable by a specific method, this is also to be understood to disclose a plant which is obtained by this method.

Where an indefinite or definite article is used when referring to a singular noun, e.g. “a”, “an” or “the”, this includes a plural of that noun unless something else is specifically stated.

The term “transgenic” means that a plant cell, plant or plant part has been altered using recombinant DNA technology to contain a nucleic acid sequence which would otherwise not be present in said plant cell, plant, or plant part or which would be expressed to a considerably lower extent. Within the scope of the present invention, the transgenic plant cell, plant or plant part contains a nucleic acid sequence selected from the group consisting of
(a) a nucleic acid sequence comprising the sequence according to any of SEQ ID Nos. 1-5, 7-9, 11-13, 15-17, 19-21, 23 and 24 or a fragment of any of these sequences;
(b) a nucleic acid sequence encoding a protein comprising the amino acid sequence according to any of SEQ ID Nos. 6, 10, 14, 18, 22 and 25;
(c) a nucleic acid sequence comprising a sequence which is at least 70% identical to the sequence according to any of SEQ ID Nos 1-5, 7-9, 11-13, 15-17, 19-21, 23 and 24 or a fragment of any of these sequences; and
(d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences;
   which is not present at the natural locus of this sequence in the genome of the control plant and/or which has been linked to sequences to which the nucleic acid sequence is not linked in the genome of the control plant and/or which is present in another 5′ to 3′ orientation compared to the orientation of this sequence in the natural locus of the control plant. Natural locus means the location on a specific chromosome, preferably the location between certain genes, more preferably the same sequence background as in the original plant which is transformed.

Preferably, the nucleic acid sequence is introduced by means of a vector. Also preferably, the nucleic acid sequence is stably integrated into the genome of the transgenic plant. In particular, the transgenic plant cell, plant or plant part of the present invention contains a nucleic acid sequence which increases the content and/or the activity of the receptor-like protein kinase compared to a control plant cell, plant or plant part. In addition to the nucleic acid sequence which increases the content and/or the activity of the receptor-like protein kinase, the transgenic plant cell, plant or plant part may contain one or more other transgenic nucleic acid sequences, for example nucleic acid sequences conferring resistance to biotic or abiotic stress and/or altering the chemical composition of the transgenic plant cell, plant or plant part. The term “transgenic” does not refer to plants having alterations in the genome which are the result of naturally occurring events, such as spontaneous mutations or of induced mutagenesis followed by breeding and selection.

The term “mutant” means that a plant cell, plant or plant part has been altered by mutagenesis so that an endogenous nucleic acid sequence selected from the group consisting of
(a) a nucleic acid sequence comprising the sequence according to any of SEQ ID Nos. 1-5, 7-9, 11-13, 15-17, 19-21, 23 and 24 or a fragment of any of these sequences;
(b) a nucleic acid sequence encoding a protein comprising the amino acid sequence according to any of SEQ ID Nos. 6, 10, 14, 18, 22 and 25;
(c) a nucleic acid sequence comprising a sequence which is at least 70% identical to the sequence according to any of SEQ ID Nos. 1-5, 7-9, 11-13, 15-17, 19-21, 23 and 24 or a fragment of any of these sequences; and
(d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences;
   contains at least one point mutation, i.e. at least one nucleotide substitution, deletion and/or addition, in comparison to the corresponding nucleic acid sequence in a control plant, plant cell or part thereof which has been used as a starting material in the mutagenesis and which has not been mutagenized. Preferably, the mutant plant contains at least one nucleotide substitution in the nucleic acid sequence according to any of SEQ ID Nos. 1-5, 7-9, 11-13, 15-17, 19-21, 23 and 24 or a fragment of any of these sequences.

The transgenic plant of the present invention may be a monocotyledonous or a dicotyledonous plant.

Examples of monocotyledonous plants are plants belonging to the genera Avena (oat), Triticum (wheat), Secale (rye), Hordeum (barley), Oryza (rice), Panicum, Pennisetum, Setaria, Sorghum (millet), Zea (maize), and the like.

Useful dicotyledonous plants comprise, inter alia, cotton, legumes, like leguminous plants and in particular alfalfa, soy bean, rape, tomato, sugar beet, potato, ornamental plants, and trees. Further useful plants can comprise fruit (in particular apples, pears, cherries, grapes, citrus, pineapple, and bananas), pumpkin, cucumber, wine, oil palms, tea shrubs, cacao trees, and coffee shrubs, tobacco, sisal, as well as, with medicinal plants, rauwolfia and digitalis.

Particularly preferred are the cereals wheat, rye, oat, barley, rice, maize and millet, sugar beet, rape, soy, tomato, potato, cotton and tobacco. Further useful plants can be taken from U.S. Pat. No. 6,137,030.

More preferably the transgenic or mutant plants are oat, barley, rye, wheat or rice plants, even more preferably the transgenic or mutant plants are barley or wheat plants and most preferably the transgenic or mutant plants are wheat plants.

Within the meaning of the present invention the term “transgenic plant” also includes the transgenic progeny of the transgenic plant and the term “mutant plant” also includes the mutant progeny of the mutant plant. The transgenic progeny of the transgenic plant comprises the nucleic acid sequence which increases the content and/or activity of the receptor-like protein kinase of the present invention. The mutant progeny of the mutant plant comprises at least one point mutation which increases the content and/or activity of the receptor-like protein kinase of the present invention. The transgenic or mutant progeny of the transgenic or mutant plant may be the result of a cross of the transgenic or mutant plant with another transgenic or mutant plant of the present invention or it may be the result of a cross with a wild-type plant or a transgenic plant having a transgene other than the transgene of the present invention. In particular, the term “transgenic plant” also comprises true breeding transgenic plants which are obtained by repeated inbreeding steps as described below.

Plant parts include, but are not limited to, stems, roots, ovules, stamens, leaves, embryos, meristematic regions, callus tissue, gametophytes, sporophytes, pollen, microspores, seeds and the like.

The term “cell” or “plant cell” as used herein refers to a single cell and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

According to the present invention, “pathogen resistance” means reducing or attenuating disease symptoms of a plant as a result of attack by a pathogen, preferably by a fungus. While said symptoms can be manifold, they preferably comprise such symptoms directly or indirectly leading to impairment of plant quality, yield quantity, or suitability for use as feed or food, or impeding sowing, cultivation, harvest, or processing of the crop. Furthermore, “resistance” also means that pests and/or a pathogen and preferably a fungus and especially preferably the fungi described below display reduced growth in a plant and reduced or absent propagation. The term “resistance” also includes a so-called transient resistance, i.e. the transgenic plants or plant cells of the present invention have an increased resistance to pests and/or pathogens or fungi compared to the corresponding control plants only for a limited period of time.

Preferably, the resistance conferred is a nonhost resistance which is the durable resistance of all known genotypes of a plant species to all known races or isolates of a pathogen species. Hence, the resistance is transferred from a plant species which is resistant to a specific pathogen to a plant species which is susceptible to said pathogen.

According to the present invention, the term “increased pathogen resistance” is understood to denote that the transgenic plants or plant cells of the present invention are infected less severely and/or less frequently by plant pathogens.

In one embodiment the reduced frequency and the reduced extent of pathogen infection, respectively, on the transgenic plants or plant cells according to the present invention is determined as compared to the corresponding control plant. According to the present invention, an increase in resistance means that an infection of the plant by the pathogen occurs less frequently or less severely by at least 5%, preferably by at least 20%, also preferably by at least 50%, 60% or 70%, especially preferably by at least 80%, 90% or 100%, also especially preferably by the factor 5, particularly preferably by at least the factor 10, also particularly preferably by at least the factor 50, and more preferably by at least the factor 100, and most preferably by at least the factor 1000, as compared to the control plant.

Alternatively, the pathogen resistance may be described by reference to a relative susceptibility index (SI) which compares the susceptibility of a plant of the present invention to a pathogen with the susceptibility of a control plant to said pathogen, the latter being set to 100%. The relative susceptibility index of the plants of the present invention is less than 90%, preferably less than 85 or 80%, more preferably less than 75 or 70% and most preferably less than 68%.

When used in connection with transgenic plants, the terms “control plant”, “control plant cell” and “control plant part” refer to a plant cell, an explant, seed, plant component, plant tissue, plant organ, or whole plant used to compare against a transgenic plant which has been modified by the method of the present invention for the purpose of identifying an enhanced phenotype or a desirable trait in the transgenic plant. A “control plant” may in some cases be a transgenic plant line that comprises an empty vector or marker gene, but does not contain the recombinant polynucleotide of interest that is present in the transgenic plant being evaluated, i.e. the nucleic acid sequence increasing the content and/or the activity of receptor-like protein kinase. A control plant may be a plant of the same line or variety as the transgenic plant being tested, or it may be of another line or variety, such as a plant known to have a specific phenotype, characteristic, or known genotype. Another suitable control plant is a genetically unaltered or non-transgenic plant of the parental line used to generate the transgenic plant of the present invention, i.e. the wild-type plant.

When used in connection with mutant plants, the terms “control plant”, “control plant cell” and “control plant part” refer to a plant cell, an explant, seed, plant component, plant tissue, plant organ, or whole plant used to compare against a mutant plant and which has been used as starting material for the mutagenization and which does not contain the at least one point mutation of the mutant plant in the nucleic acid sequence of the present invention.

The infection of test plants with pathogens such as fungi in order to examine potential resistance phenomena is a method well-known to those skilled in the art. The test plants used must be responsive to the pathogen used, i.e. they must be able to serve as host plant for said pathogen, and the pathogen attack must be detectable by simple means. Preferred test plants are wheat or barley plants, which are, for example, inoculated with the powdery mildew fungus Blumeria graminis, preferably with the corresponding forma specialis of the plant to be inoculated, i.e. the pathogen which is adapted to the specific host used. Hence, wheat is preferably inoculated with Blumeria graminis f.sp. tritici and barley is preferably inoculated with Blumeria graminis f.sp. hordei. “Inoculating” denotes contacting the plant with the fungus the plant is to be infected with, or with infectious parts thereof, under conditions in which the fungus may enter a wild-type plant.

The fungal infestation of the plant may then be evaluated by means of a suitable evaluation procedure. The visual inspection, in which the formed fungal structures are detected in the plant and quantified, is particularly suitable. In order to identify successfully transformed cells in transient experiments, a reporter gene, such as the beta-glucuronidase (GUS) gene from E. coli, a fluorescence gene, the green fluorescence protein (GFP) gene from Aequorea victoria, the luciferase gene from Photinus pyralis or the beta-galactosidase (lacZ) gene from E. coli, the expression of which in the plant cells may be proven by simple methods, is co-transformed in a suitable vector with the vector mediating the expression of the receptor-like protein kinase. Optionally, the formed fungal structures may be stained by methods well-known to those skilled in the art in order to improve the determination thereof, e.g. by staining with coomassie or trypan blue. Then, the number of infected plants transformed with the nucleic acid molecule to be tested is compared to the number of infected wild-type or control plants and the degree of pathogen resistance is calculated. Alternatively, fungal resistance may be scored by determining the symptoms of fungal infection on the infected plant, for example by eye, and calculating the diseased leaf area, The diseased leaf area is the percentage of the leaf area showing symptoms of fungal infection, such as fungal pycnidia or fungal colonies. The diseased leaf area of infected plants transformed with the vector mediating the expression of the receptor-like protein kinase is lower than the diseased leaf area of infected control plants. Preferably, the diseased leaf area of infected plants transformed with the vector mediating the expression of the receptor-like protein kinase is 90%, 85%, 80%, 75% or 70%, more preferably it is 65%, 60%, 55% or 50%, even more preferably it is 45%, 40%, 35% or 30% and most preferably it is 25%, 20%, 15% or 10% of the diseased leaf area of the infected control plants.

According to the present invention, the term “plant pathogens” includes viral, bacterial, fungal and other pathogens. Preferably, the term “plant pathogens” comprises fungal pathogens.

According to the present invention, the term “plant pathogens” includes biotrophic, hemibiotrophic and necrotrophic pathogens. Preferably, the plant pathogen is a biotrophic pathogen, more preferably a biotrophic fungal pathogen.

The biotrophic phytopathogenic fungi, such as many rusts, depend for their nutrition on the metabolism of living cells of the plants. This type of fungi belongs to the group of biotrophic fungi, like other rust fungi, powdery mildew fungi or oomycete pathogens like the genus Phytophthora or Peronopora. The necrotrophic phytopathogenic fungi, e.g. species from the genus Fusarium, Rhizoctonia or Mycospaerella, depend for their nutrition on dead cells of the plants. Soybean rust has occupied an intermediate position, since it penetrates the epidermis directly, whereupon the penetrated cell becomes necrotic. After the penetration, the fungus changes over to an obligatory-biotrophic lifestyle. The subgroup of the biotrophic fungal pathogens which follows essentially such an infection strategy is hemibiotrophic.

20

TABLE 1
Diseases caused by biotrophic phytopathogenic fungi

| Disease | Pathogen |
|---|---|
| Black rust | Puccinia triticina |
| Leaf rust | Puccinia recondita |
| Yellow rust | Puccinia striiformis |
| Powdery mildew | Erysiphe graminis/Blumeria graminis |
| Rust (common corn) | Puccinia sorghi |
| Rust (Southern corn) | Puccinia polysora |
| Tobacco leaf spot | Cercospora nicotianae |
| Rust (soybean) | Phakopsora pachyrhizi, P. meibomiae |
| Rust (tropical corn) | Physopella pallescens, P. zeae = |
| | Angiopsora zeae |

TABLE 2
Diseases caused by necrotrophic and/or hemibiotrophic fungi and Oomycetes

| Disease | Pathogen |
|---|---|
| Plume blotch | Septoria (Stagonospora) nodorum |
| Leaf blotch | Septoria tritici |
| Ear fusarioses | Fusarium spp. |
| Eyespot | Pseudocercosporella herpotrichoides |
| Smut | Ustilago spp. |
| Late blight | Phytophthora infestans |
| Bunt | Tilletia caries |
| Take-all | Gaeumannomyces graminis |
| Anthrocnose leaf blight | Colletotrichum graminicola (teleomorph: |
| Anthracnose stalk rot | Glomerella graminicola Politis); Glomerella |
| | tucumanensis (anamorph: Glomerella falcatum |
| | Went) |
| Aspergillus ear and | Aspergillus flavus |
| kernel rot | |
| Banded leaf and sheath spot | Rhizoctonia solani Kuhn = Rhizoctonia |
| | microsclerotia J. Matz (telomorph: |
| | Thanatephorus cucumeris) |
| Black bundle disease | Acremonium strictum W. Gams = |
| | alosporium acremonium Auct. non Corda |
| Black kernel rot | Lasiodiplodia theobromae = |
| | Botryodiplodia theobromae |
| Borde blanco | Marasmiellus sp. |
| Brown spot (black spot, stalk rot) | Physoderma maydis |
| Cephalosporium kernel rot | Acremonium strictum = Cephalosporium |
| | acremonium |
| Charcoal rot | Macrophomina phaseolina |
| Corticium ear rot | Thanatephorus cucumeris = |
| | Corticium sasakii |
| Curvularia leaf spot | Curvularia clavata, C. eragrostidis, = C. maculans |
| | (teleomorph: Cochliobolus |
| | eragrostidis), Curvularia inaequalis, C. intermedia |
| | (teleomorph: Cochliobolus |
| | intermedius), Curvularia lunata (teleomorph: |
| | Cochliobolus lunatus), Curvularia pallescens |
| | (teleomorph: Cochliobolus pallescens), |
| | Curvularia senegalensis, C. tuberculata |
| | (teleomorph: Cochliobolus tuberculatus) |
| Didymella leaf spot | Didymella exitalis |
| Diplodia ear and stalk rot | Diplodia frumenti (teleomorph: Botryosphaeria |
| | festucae) |
| Diplodia ear and stalk rot, seed rot | Diplodia maydis = |
| and seedling blight | Stenocarpella maydis |
| Diplodia leaf spot or streak | Stenocarpella macrospora = |
| | Diplodialeaf macrospora |
| Brown stripe downy | Sclerophthora rayssiae var. zeae |
| mildew | |
| Crazy top downy mildew | Sclerophthora macrospora = |
| | Sclerospora macrospora |
| Green ear downy mildew (graminicola | Sclerospora graminicola |
| downy mildew) | |
| Dry ear rot (cob, | Nigrospora oryzae |
| kernel and stalk rot) | (teleomorph: Khuskia oryzae) |
| Ear rots (minor) | Alternaria altemata = A. tenuis, |
| | Aspergillus glaucus, A. niger, |
| | Aspergillus spp., Botrytis cinerea (teleomorph: |
| | Botryotinia fuckeliana), Cunninghamella sp., |
| | Curvularia pallescens, |
| | Doratomyces stemonitis = |
| | Cephalotrichum stemonitis, |
| | Fusarium culmorum, |
| | Gonatobotrys simplex, |
| | Pithomyces maydicus, |
| | Rhizopus microsporus Tiegh., |
| | R. stolonifer = R. nigricans, |
| | Scopulariopsis brumptii |
| Ergot (horse's tooth) | Claviceps gigantea |
| | (anamorph: Sphacelia sp.) |
| Eyespot | Aureobasidium zeae = Kabatiella zeae |
| Fusarium ear and stalk rot | Fusarium subglutinans = |
| | F. moniliforme var.subglutinans |
| Fusarium kernel, root and stalk rot, | Fusarium moniliforme |
| seed rot and seedling blight | (teleomorph: Gibberella fujikuroi) |
| Fusarium stalk rot, | Fusarium avenaceum |
| seedling root rot | (teleomorph: Gibberella avenacea) |
| Gibberella ear and stalk rot | Gibberella zeae |
| | (anamorph: Fusarium graminearum) |
| Gray ear rot | Botryosphaeria zeae = Physalospora zeae |
| | (anamorph: Macrophoma zeae) |
| Gray leaf spot | Cercospora sorghi = C. sorghi var. maydis, C. zeae- |
| (Cercospora leaf spot) | maydis |
| Helminthosporium root rot | Exserohilum pedicellatum = Helminthosporium |
| | pedicellatum (teleomorph: Setosphaeria |
| | pedicellata) |
| Hormodendrum ear rot | Cladosporium cladosporioides = |
| (Cladosporium rot) | Hormodendrum cladosporioides, C. herbarum |
| | (teleomorph: Mycosphaerella tassiana) |
| Leaf spots, minor | Alternaria alternata, |
| | Ascochyta maydis, A. tritici, |
| | A. zeicola, Bipolaris victoriae = |
| | Helminthosporium victoriae |
| | (teleomorph: Cochliobolus victoriae), C. sativus |
| | (anamorph: Bipolaris sorokiniana = H. sorokinianum = |
| | H. sativum), Epicoccum nigrum, |
| | Exserohilum prolatum = Drechslera prolata |
| | (teleomorph: Setosphaeria prolata) |
| | Graphium penicillioides, |
| | Leptosphaeria maydis, Leptothyrium zeae, |
| | Ophiosphaerella herpotricha, (anamorph: |
| | Scolecosporiella sp.), |
| | Paraphaeosphaeria michotii, Phoma sp., |
| | Septoria zeae, S. zeicola, |
| | S. zeina |
| Northern corn leaf blight (white blast, | Setosphaeria turcica (anamorph: Exserohilum |
| crown stalk rot, stripe) | turcicum = Helminthosporium turcicum) |
| Northern corn leaf spot | Cochliobolus carbonum (anamorph: Bipolaris |
| Helminthosporium ear rot (race 1) | zeicola = Helminthosporium carbonum) |
| Penicillium ear rot (blue eye, blue | Penicillium spp., P. chrysogenum, |
| mold) | P. expansum, P. oxalicum |
| Phaeocytostroma stalk and root rot | Phaeocytostroma ambiguum, = |
| | Phaeocytosporella zeae |
| Phaeosphaeria leaf spot | Phaeosphaeria maydis = Sphaerulina maydis |
| Physalospora ear rot (Botryosphaeria | Botryosphaeria festucae = Physalospora |
| ear rot) | zeicola (anamorph: Diplodia frumenti) |
| Purple leaf sheath | Hemiparasitic bacteria and fungi |
| Pyrenochaeta stalk and root rot | Phoma terrestris = |
| | Pyrenochaeta terrestris |
| Pythium root rot | Pythium spp., P. arrhenomanes, |
| | P. graminicola |
| Pythium stalk rot | Pythium aphanidermatum = |
| | P. butleri L. |
| Red kernel disease (ear mold, leaf | Epicoccum nigrum |
| and seed rot) | |
| Rhizoctonia ear rot (sclerotial rot) | Rhizoctonia zeae (teleomorph: Waitea |
| | circinata) |
| Rhizoctonia root and stalk rot | Rhizoctonia solani, Rhizoctonia zeae |
| Root rots (minor) | Alternaria alternata, Cercospora sorghi, |
| | Dictochaeta fertilis, Fusarium acuminatum |
| | (teleomorph: Gibberella acuminata), F. equiseti |
| | (teleomorph: G. intricans), F. oxysporum, |
| | F. pallidoroseum, F. poae, F. roseum, G. cyanogena, |
| | (anamorph: F. sulphureum), |
| | Microdochium bolleyi, Mucor sp., Periconia |
| | circinata, Phytophthora cactorum, P. drechsleri, |
| | P. nicotianae var. parasitica, Rhizopus arrhizus |
| Rostratum leaf spot | Setosphaeria rostrata, (anamorph: |
| (Helminthosporium leaf disease, ear | xserohilum rostratum = Helminthosporium |
| and stalk rot) | rostratum) |
| Java downy mildew | Peronosclerospora maydis = |
| | Sclerospora maydis |
| Philippine downy mildew | Peronosclerospora philippinensis = Sclerospora |
| | philippinensis |
| Sorghum downy mildew | Peronosclerospora sorghi = |
| | Sclerospora sorghi |
| Spontaneum downy mildew | Peronosclerospora spontanea = |
| | Sclerospora spontanea |
| Sugarcane downy mildew | Peronosclerospora sacchari = |
| | Sclerospora sacchari |
| Sclerotium ear rot (southern blight) | Sclerotium rolfsii Sacc. (teleomorph: Athelia |
| | rolfsii) |
| Seed rot-seedling blight | Bipolaris sorokiniana, B. zeicola = |
| | Helminthosporium carbonum, Diplodia maydis, |
| | Exserohilum pedicillatum, Exserohilum turcicum = |
| | Helminthosporium turcicum, Fusarium |
| | avenaceum, F. culmorum, F. moniliforme, |
| | Gibberella zeae (anamorph: F. graminearum), |
| | Macrophomina phaseolina, Penicillium spp., |
| | Phomopsis sp., Pythium spp., Rhizoctonia |
| | solani, R. zeae, Sclerotium rolfsii, Spicaria sp. |
| Selenophoma leaf spot | Selenophoma sp. |
| Sheath rot | Gaeumannomyces graminis |
| Shuck rot | Myrothecium gramineum |
| Silage mold | Monascus purpureus, M ruber |
| Smut, common | Ustilago zeae = U. maydis |
| Smut, false | Ustilaginoidea virens |
| Smut, head | Sphacelotheca reiliana = Sporisorium |
| | holcisorghi |
| Southern corn leaf blight and stalk rot | Cochliobolus heterostrophus (anamorph: |
| | Bipolaris maydis = Helminthosporium maydis) |
| Southern leaf spot | Stenocarpella macrospora = Diplodia |
| | macrospora |
| Stalk rots (minor) | Cercospora sorghi, Fusarium episphaeria, F. merismoides, |
| | F. oxysporum Schlechtend, F. poae, |
| | F. roseum, F. solani (teleomorph: Nectria |
| | haematococca), F. tricinctum, Mariannaea |
| | elegans, Mucor sp., Rhopographus zeae, |
| | Spicaria sp. |
| Storage rots | Aspergillus spp., Penicillium spp. und weitere |
| | Pilze |
| Tar spot | Phyllachora maydis |
| Trichoderma ear rot and root rot | Trichoderma viride = T. lignorum teleomorph: |
| | Hypocrea sp. |
| White ear rot, root and stalk rot | Stenocarpella maydis = Diplodia zeae |
| Yellow leaf blight | Ascochyta ischaemi, Phyllosticta maydis |
| | (teleomorph: Mycosphaerella zeae-maydis) |
| Zonate leaf spot | Gloeocercospora sorghi |

Preferably, fungal pathogens or fungal-like pathogens (like for example Chromista) are from the group comprising Plasmodiophoromycetes, Oomycetes, Ascomycetes, Chytridiomycetes, Zygomycetes, Basidiomycetes, and Deuteromycetes (Fungi imperfecti). The fungal pathogens listed in Tables 1 and 2 as well as the diseases associated therewith are to be mentioned in an exemplary, yet not limiting manner.

Particularly preferred are:
Plasmodiophoromycetes like Plasmodiophora brassicae (clubroot of crucifers), Spongospora subterranea (powdery scab of potato tubers), Polymyxa graminis (root disease of cereals and grasses),
Oomycetes like Bremia lactucae (downy mildew of lettuce), Peronospora (downy mildew) of snapdragon (P. antirrhini), onion (P. destructor), spinach (P. effusa), soy bean (P. manchurica), tobacco (“blue mold”, P. tabacina) alfalfa and clover (P. trifolium), Pseudoperonospora humuli (downy mildew of hop), Plasmopara (downy mildew) of grapes (P. viticola) and sun flower (P. halstedii), Sclerophthora macrospora (downy mildew of cereals and grasses), Pythium (seed rot, seedling damping-off, and root rot and all types of plants, for example black root disease of beet caused by P. debaryanum), Phytophthora infestans (potato light blight, tomato late blight, etc.), Albugo spec. (white rust on cruciferous plants)
Ascomycetes like Microdochium nivale (snow mold of rye and wheat), Fusarium graminearum, Fusarium culmorum (head blight, in particular of wheat), Fusarium oxysporum (fusarium wilt of tomato), Blumeria graminis (powdery mildew of barley (f. sp. hordei) and wheat (f. sp. tritici)), Erysiphe pisi (pea mildew), Nectria galligena (Nectria canker of fruit trees), Unicnula necator (grapevine powdery mildew), Pseudopeziza tracheiphila (grapevine red fire disease), Claviceps purpurea (ergot on, for example, rye and grasses), Gaeumannomyces graminis (black leg disease of wheat, rye and, inter alia, grasses), Magnaporthe grisea (rice blast disease), Pyrenophora graminea (leaf stripe disease of barley), Pyrenophora teres (net blotch disease of barley), Pyrenophora tritici-repentis (tan spot disease) Septoria tritici (leaf spot of wheat), Venturia inaequalis (apple scab disease), Sclerotinia sclerotium (white mold, stem canker of rape), Pseudopeziza medicaginis (leaf spot diseases of lucerne, white and red clover).
Basidiomycetes like Typhula incarnata (typhula snow mold of barley, rye, and wheat), Ustilago maydis (corn smut), Ustilago nuda (loose smut of barley), Ustilago tritici (loose smut of wheat and spelt), Ustilago avenae (loose smut of oat), Rhizoctonia solani (taproot lesions of potatoes), Sphacelotheca spp. (head smut of sorghum), Melampsora lini (rust of flax), Puccinia graminis (stem rust of wheat, barley, rye, oat), Puccinia recondita (brown rust of wheat), Puccinia triticina (wheat leaf rust), Puccinia dispersa (brown rust of rye), Puccinia hordei (brown rust of barley), Puccinia coronata (crown rust of oat), Puccinia striiformis (yellow rust of wheat, barley, rye, and various grasses), Uromyces appendiculatus (bean rust), Phakopsora pachyrhizi (Asian soybean rust), Sclerotium rolfsii (root and stem rots of many plants).
Deuteromycetes (Fungi imperfecti) like Septoria nodorum (glume blotch) of wheat (Septoria tritici), Pseudocercosporella herpotrichoides (stem break disease in wheat, barley, rye), Rynchosporium secalis (scald disease in rye and barley), Alternaria solani (early blight of potato and tomato), Phoma betae (black rot of beet), Cercospora beticola (Cercospora leaf spot of beet), Alternaria brassicae (dark leaf spot of rape, cabbage and other cruciferous plants), Verticillium dahliae (Verticillium wilt and stalk rot of rape), Colletotrichum lindemuthianum (bean anthracnose), Phoma lingam-phoma stem canker (black leg disease of cabbage; crown and stem canker of rape), Botrytis cinerea (gray mold diseases of grapevine, strawberry, tomato, hop, etc.).

Likewise preferred are: Phytophthora infestans (late blight of tomato, root and foot rot of tomato, etc.), Microdochium nivale (formerly Fusarium nivale; snow mold of rye and wheat), Fusarium graminearum, Fusarium culmorum (head blight of wheat), Fusarium oxysporum (Fusarium wilt of tomato), Blumeria graminis (powdery mildew of barley (f. sp. hordei) and wheat (f. sp. tritici)), Puccinia triticina (wheat leaf rust), Magnaporthe grisea (rice blast disease), Sclerotinia sclerotium (white mold, stem canker of rape), Septoria nodorum and Septoria tritici (glume blotch of wheat), Alternaria brassicae (dark leaf spot of rape, cabbage and other cruciferous plants), Phakopsora pachyrhizi (Asian soybean rust), Phoma lingam (phoma stem canker, black leg disease of cabbage; crown and stem canker of rape).

The pathogens listed in Table 3 as well as the diseases associated therewith are to be mentioned as bacterial pathogens in an exemplary, yet not limiting manner.

TABLE 3
Bacterial diseases

| Disease | Pathogen |
|---|---|
| Bacterial leaf blight | Pseudomonas avenae subsp. avenae |
| and stalk rot | |
| Bacterial leaf spot | Xanthomonas campestris pv. holcicola |
| Bacterial stalk rot | Enterobacter dissolvens = |
| | Erwinia dissolvens |
| Bacterial stalk and top rot | Erwinia carotovora subsp. carotovora, |
| | Erwinia chrysanthemi pv. zeae |
| Bacterial stripe | Pseudomonas andropogonis |
| Chocolate spot | Pseudomonas syringae pv. coronafaciens |
| Goss's bacterial | Clavibacter michiganensis subsp. |
| wilt and blight (leaf | nebraskensis = |
| freckles and wilt) | Corynebacterium michiganense |
| | pv. andnebraskense |
| Holcus spot | Pseudomonas syringae pv. syringae |
| Purple leaf sheath | Hemiparasitic bacteria |
| Seed rot-seedling blight | Bacillus subtilis |
| Stewart's disease | Pantoea stewartii = |
| (bacterial wilt) | Erwinia stewartii |
| Corn stunt (achapparramiento, | Spiroplasma kunkelii |
| maize stunt, Mesa Central | |
| or Rio Grande maize stunt) | |

Particularly preferably, the transgenic plants produced according to the present invention are resistant to the following pathogenic bacteria:

Corynebacterium sepedonicum (bacterial ring rot of potato), Erwinia carotovora (black leg rot of potato), Erwinia amylovora (fire blight of pear, apple, quince), Streptomyces scabies (common scab of potato), Pseudomonas syringae pv. tabaci (wild fire disease of tobacco), Pseudomonas syringae pv. phaseolicola (halo blight disease of dwarf bean), Pseudomonas syringae pv. tomato (“bacterial speck” of tomato), Xanthomonas campestris pv. malvacearum (angular leaf spot of cotton), and Xanthomonas campestris pv. oryzae (bacterial blight of rice and other grasses).

The term “viral pathogens” includes all plant viruses, like for example tobacco or cucumber mosaic virus, ringspot virus, necrosis virus, maize dwarf mosaic virus, etc.

The pathogens listed in Table 4 as well as the diseases associated therewith are to be mentioned as viral pathogens in an exemplary, yet not limiting manner.

TABLE 4
Viral diseases

| Disease | Pathogen |
|---|---|
| American wheat striate | American wheat striate mosaic virus (AWSMV) |
| (wheat striate mosaic) | |
| Barley stripe mosaic | Barley stripe mosaic virus (BSMV) |
| Barley yellow dwarf | Barley yellow dwarf virus (BYDV) |
| Brome mosaic | Brome mosaic virus (BMV) |
| Cereal chlorotic mottle | Cereal chlorotic mottle virus (CCMV) |
| Corn chlorotic vein banding | Corn chlorotic vein banding virus (CCVBV) |
| (Braizilian maize mosaic) | |
| Corn lethal necrosis | Virus complex from Maize chlorotic mottle virus |
| | (MCMV) and Maize dwarf mosaic virus (MDMV) A |
| | or B or Wheat streak mosaic virus(WSMV) |
| Cucumber mosaic | Cucumber mosaic virus (CMV) |
| Cynodon chlorotic streak | Cynodon chlorotic streak virus (CCSV) |
| Johnsongrass mosaic | Johnsongrass mosaic virus (JGMV) |
| Maize bushy stunt | Mycoplasma-like organism (MLO) associated |
| Maize chlorotic dwarf | Maize chlorotic dwarf virus (MCDV) |
| Maize chlorotic mottle | Maize chlorotic mottle virus (MCMV) |
| Maize dwarf mosaic | Maize dwarf mosaic virus (MDMV) strains A, D, E |
| | and F |
| Maize leaf fleck | Maize leaf fleck virus (MLFV) |
| Maize line | Maize line virus (MLV) |
| Maize mosaic (corn leaf stripe, | Maize mosaic virus (MMV) |
| enanismo rayado) | |
| Maize mottle and chlorotic stunt | Maize mottle and chlorotic stunt virus |
| Maize pellucid ringspot | Maize pellucid ringspot virus (MPRV) |
| Maize raya gruesa | Maize raya gruesa virus (MRGV) |
| maize rayado fino (fine striping | Maize rayado fino virus (MRFV) |
| disease) | |
| Maize red leaf and red stripe | Mollicute |
| Maize red stripe | Maize red stripe virus (MRSV) |
| Maize ring mottle | Maize ring mottle virus (MRMV) |
| Maize rio IV | Maize rio cuarto virus (MRCV) |
| Maize rough dwarf | Maize rough dwarf virus (MRDV) |
| (nanismo ruvido) | (Cereal tillering disease virus) |
| Maize sterile stunt | Maize sterile stunt virus |
| | (strains of barley yellow striate virus) |
| Maize streak | Maize streak virus (MSV) |
| Maize stripe (maize chlorotic | Maize stripe virus |
| stripe, maize hoja blanca) | |
| Maize stunting | Maize stunting virus |
| Maize tassel abortion | Maize tassel abortion virus (MTAV) |
| Maize vein enation | Maize vein enation virus (MVEV) |
| Maize wallaby ear | Maize wallaby ear virus (MWEV) |
| Maize white leaf | Maize white leaf virus |
| Maize white line mosaic | Maize white line mosaic virus (MWLMV) |
| Millet red leaf | Millet red leaf virus (MRLV) |
| Northern cereal mosaic | Northern cereal mosaic virus (NCMV) |
| Oat pseudorosette (zakuklivanie) | Oat pseudorosette virus |
| Oat sterile dwarf | Oat sterile dwarf virus (OSDV) |
| Rice black-streaked dwarf | Rice black-streaked dwarf virus (RBSDV) |
| Rice stripe | Rice stripe virus (RSV) |
| Sorghum mosaic | Sorghum mosaic virus (SrMV) (also: sugarcane |
| | mosaic virus (SCMV) strains H, I and M) |
| Sugarcane Fiji disease | Sugarcane Fiji disease virus (FDV) |
| Sugarcane mosaic | Sugarcane mosaic virus (SCMV) strains A, B, D, E, |
| | SC, BC, Sabi and MB (formerly MDMV-B) |
| Wheat spot mosaic | Wheat spot mosaic virus (WSMV) |

The plants and plant cells according to the present invention can also be resistant to animal pests like insects and nematodes. Insects, like for example beetles, caterpillars, lice, or mites are to be mentioned in an exemplary, yet not limiting manner.

Preferably, the plants according to the present invention are resistant to insects of the species of Coleoptera, Diptera, Hymenoptera, Lepidoptera, Mallophaga, Homoptera, Hemiptera, Orthoptera, Thysanoptera. Dermaptera, Isoptera, Anoplura, Siphonaptera, Trichoptera, etc. Insects of the following species are particularly preferred: Coleoptera and Lepidoptera, like, for example, the European corn borer (ECB), Diabrotica barberi (Northern corn rootworm), Diabrotica undecimpunctata (Southern corn rootworm), Diabrotica virgifera (Western corn rootworm), Agrotis ipsilon (black cutworm), Crymodes devastator (glassy cutworm), Feltia ducens (dingy cutworm), Agrotis gladiaria (claybacked cutworm), Melanotus spp., Aeolus mellillus (wireworm), Aeolus mancus (wheat wireworm), Horistonotus uhlerii (sand wireworm), Sphenophorus maidis (maize billbug), Sphenophorus zeae (timothy billbug), Sphenophorus parvulus (bluegrass billbug), Sphenophorus callosus (southern corn billbug), Phyllogphaga spp. (white grubs), Anuraphis maidiradicis (corn root aphid), Delia platura (seedcorn maggot), Colaspis brunnea (grape colaspis), Stenolophus lecontei (seedcorn beetle), and Clivinia impressifrons (lender seedcorn beetle).

Furthermore, there are to be mentioned: the cereal leaf beetle (Oulema melanopus), the frit fly (Oscinella kit), wireworms (Agrotis lineatus), and aphids (like for example the bird cherry-oat aphid Rhopalosiphum padi, the grain aphid Sitobion avenae).

The pathogens listed in Table 5 as well as the diseases associated therewith are to be mentioned as nematode pests in an exemplary, yet not limiting manner.

TABLE 5
Parasitic nematodes

| Damage | Pathogenic nematode |
|---|---|
| Awl | Dolichodorus spp., D. heterocephalus |
| Bulb and stem nematode, | Ditylenchus dipsaci |
| beet eelworm | |
| (“Bulb and stem”; | |
| Europe) | |
| Burrowing | Radopholus similis |
| Cereal cyst nematode | Heterodera avenae, H. zeae, |
| (“Cyst”) | Punctodera chalcoensis |
| Dagger | Xiphinema spp., X. americanum, |
| | X. mediterraneum |
| False root-knot | Nacobbus dorsalis |
| Lance, Columbia | Hoplolaimus columbus |
| Lance | Hoplolaimus spp., H. galeatus |
| Lesion | Pratylenchus spp., P. brachyurus, |
| | P. crenatus, P. hexincisus, P. neglectus, |
| | P. penetrans, P. scribneri, P. thornei, P. zeae |
| Needle | Longidorus spp., L. breviannulatus |
| Ring | Criconemella spp., C. ornata |
| Root-knot nematode | Meloidogyne spp., M. chitwoodi, |
| | M. incognita, M. javanica |
| Spiral | Helicotylenchus spp. |
| Sting | Belonolaimus spp., B. longicaudatus |
| Stubby-root | Paratrichodorus spp., P. christiei, P. minor, |
| | Quinisulcius acutus, Trichodorus spp. |
| Stunt | Tylenchorhynchus dubius |

Particularly preferably, the transgenic plants produced according to the present invention are resistant to Globodera rostochiensis and G. pallida (cyst nematodes of potato, tomato, and other solanaceae), Heterodera schachtii (beet cyst nematodes of sugar and fodder beets, rape, cabbage, etc.), Heterodera avenae (cereal cyst nematode of oat and other types of cereal), Ditylenchus dipsaci (bulb and stem nematode, beet eelworm of rye, oat, maize, clover, tobacco, beet), Anguina tritici (wheat seed gall nematode), seed galls of wheat (spelt, rye), Meloidogyne hapla (root-knot nematode of carrot, cucumber, lettuce, tomato, potato, sugar beet, lucerne).

In individual species of particular agricultural importance, the plants according to the present invention are preferably resistant to the following pathogens:

In barley, the plants are resistant to the fungal, bacterial, and viral pathogens Puccinia hordei (barley stem rust), Blumeria (Erysiphe) graminis f. sp. hordei (barley powdery mildew), Rhynchosporium secalis (barley scald), barley yellow dwarf virus (BYDV), and the pathogenic insects/nematodes Ostrinia nubilalis (European corn borer); Agrotis ipsilon (black cutworm); Schizaphis graminum (greenbug); Blissus leucopterus (chinch bug); Acrosternum hilare (green stink bug); Euschistus servus (brown stink bug); Deliaplatura (seedcorn maggot); Mayetiola destructor (Hessian fly); Petrobia latens (brown wheat mite).

In soy bean, the plants are resistant to the fungal, bacterial, or viral pathogens Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium roffsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffussa, Fusarium semitectum, Phialophora gregata, soy bean mosaic virus, Glomerella glycines, tobacco ring spot virus, tobacco streak virus, Phakopsora pachyrhizi, Phakopsora meibomiae, Pythium aphanidermatum, Pythium ultimum, Pythium debaryanum, tomato spotted wilt virus, Heterodera glycines, Fusarium solani and the pathogenic insects/nematodes Pseudoplusia includens (soybean looper); Anticarsia gemmatalis (velvetbean caterpillar); Plathypena scabra (green cloverworm); Ostrinia nubilalis (European corn borer); Agrotis ipsilon (black cutworm); Spodoptera exigua (beet armyworm); Heliothis virescens (cotton budworm); Helicoverpa zea (cotton bollworm); Epilachna varivestis (Mexican bean beetle); Myzus persicae (green peach aphid); Empoasca fabae (potato leaf hopper); Acrosternum hilare (green stink bug); Melanoplus femurrubrum (redlegged grasshopper); Melanoplus differentialis (differential grasshopper); Hylemya platura (seedcom maggot); Sericothrips variabilis (soybean thrips); Thrips tabaci (onion thrips); Tetranychus turkestani (strawberry spider mite); Tetranychus urticae (twospotted spider mite).

In canola, the plants are resistant to the fungal, bacterial, or viral pathogens Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum and Alternaria altemata.

In alfalfa, the plants are resistant to the fungal, bacterial, or viral pathogens Clavibacter michiganensis subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae.

In wheat, the plants are resistant to the fungal, bacterial, or viral pathogens Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Blumeria (Erysiphe) graminis f. sp. tritici, Puccinia graminis f. sp. tritici, Puccinia recondita f. sp. tritici, Puccinia striiformis, Puccinia triticina, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Pythium gramicola, High Plains Virus, European wheat striate virus and to the pathogenic insects/nematodes Pseudaletia unipunctata (army worm); Spodoptera frugiperda (fall armyworm); Elasmopalpus lignosellus (lesser cornstalk borer); Agrotis orthogonia (western cutworm); Elasmopalpus Zignosellus (lesser cornstalk borer); Oulema melanopus (cereal leaf beetle); Hypera punctata (clover leaf weevil); Diabrotica undecimpunctata howardi (southern corn rootworm); Russian wheat aphid; Schizaphis graminum (greenbug); Macrosiphum avenae (English grain aphid); Melanoplus femurrubrum (redlegged grasshopper); Melanoplus differentialis (differential grasshopper); Melanoplus sanguinipes (migratory grasshopper); Mayetiola destructor (Hessian fly); Sitodiplosis mosellana (wheat midge); Meromyza americana (wheat stem maggot); Hylemya coarctata (wheat bulb fly); Frankliniella fusca (tobacco thrips); Cephus cinctus (wheat stem sawfly); Aceria tulipae (wheat curl mite).

In sun flower, the plants are resistant to the fungal, bacterial, or viral pathogens Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum p.v. Carotovora, Cephalosporium acremonium, Phytophthora cryptogea, Albugo tragopogonis and to the pathogenic insects/nematodes Suleima helianthana (sunflower bud moth); Homoeosoma electellum (sunflower moth); Zygogramma exclamationis (sunflower beetle); Bothyrus gibbosus (carrot beetle); Neolasioptera murtfeldtiana (sunflower seed midge).

In maize, the plants are resistant to the fungal, bacterial, or viral pathogens Fusarium moniliforme var. subglutinans, Erwinia stewartii, Fusarium moniliforme, Gibberella zeae (Fusarium graminearum), Stenocarpella maydis (Diplodia maydis), Pythium irregulare, Pythium debaryanum, Pythium graminicola, Pythium splendens, Pythium ultimum, Pythium aphanidermatum, Aspergillus flavus, Bipolaris maydis 0, T (Cochliobolus heterostrophus), Helminthosporium carbonum I, II & Ill (Cochliobolus carbonum), Exserohilum turcicum I, II & Ill, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi p.v. Zea, Erwinia corotovora, Cornstunt spiroplasma, Diplodia macrospora, Sclerophthora macrospora, Peronosclerospora sorghi, Peronosclerospora philippinesis, Peronosclerospora maydis, Peronosclerospora sacchari, Spacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus (MSV, Maisstrichel-Virus), Maize Stripe Virus, Maize Rough Dwarf Virus, and the pathogenic insects/nematodes Ostrinia nubilalis (European corn borer); Agrotis ipsilon (black cutworm); Helicoverpa zea (corn earworm); Spodoptera frugiperda. (fall armyworm); Diatraea grandiosella (southwestern corn borer); Elasmopalpus lignosellus (lesser cornstalk borer); Diatraea saccharalis (surgarcane borer); Diabrotica virgifera (western corn rootworm); Diabrotica longicomis barberi (northern corn rootworm); Diabrotica undecimpunctata howardi (southern corn rootworm); Melanotus spp. (wireworms); Cyclocephala borealis (northern masked chafer; white grub); Cyclocephala immaculata (southern masked chafer; white grub); Popillia japonica (Japanese beetle); Chaetocnema pulicaria (corn flea beetle); Sphenophorus maidis (maize billbug); Rhopalosiphum maidis (corn leaf aphid); Anuraphis maidiradicis (corn root aphid); Blissus leucopterus leucopterus (chinch bug); Melanoplus femurrubrum (redlegged grasshopper); Melanoplus sanguinipes (migratory grasshopper); Hylemva platura (seedcom maggot); Agromyza parvicornis (corn blot leafminer); Anaphothrips obscurus (grass thrips); Solenopsis milesta (thief ant); Tetranychus urticae (twospotted spider mite).

In sorghum, the plants are resistant to the fungal, bacterial, or viral pathogens Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola and to the pathogenic insects/nematodes Chilo partellus (sorghum borer); Spodoptera frugiperda (fall armyworm); Helicoverpa zea (corn earworm); Elasmopalpus lignosellus (lesser cornstalk borer); Feltia subterranea (granulate cutworm); Phyllophaga crinita (white grub); Eleodes, Conoderus and Aeolus spp. (wireworm); Oulema melanopus (cereal leaf beetle); Chaetocnema pulicaria (corn flea beetle); Sphenophorus maidis (maize billbug); Rhopalosiphum maidis (corn leaf aphid); Siphaflava (yellow sugarcane aphid); Blissus leucopterus leucopterus (chinch bug); Contarinia sorghicola (sorghum midge); Tetranychus cinnabarinus (carmine spider mite); Tetranychus urticae (two-spotted spider mite).

In cotton, the plants are resistant to the pathogenic insects/nematodes: Heliothis virescens (cotton budworm); Helicoverpa zea (cotton bollworm); Spodoptera exigua (beet armyworm); Pectinophora gossypiella (pink bollworm); Anthonomus grandis grandis (boll weevil); Aphis gossypii (cotton aphid); Pseudatomoscelis seriatus (cotton fleahopper); Trialeurodes abutilonea (bandedwinged whitefly); Lygus lineolaris (tarnished plant bug); Melanoplus femurrubrum (redlegged grasshopper); Melanoplus differentialis (differential grasshopper); Thrips tabaci (onion thrips); Franklinkiella fusca (tobacco thrips); Tetranychus cinnabarinus (carmine spider mite); Tetranychus urticae (two-spotted spider mite).

In rice, the plants are resistant to the pathogenic insects/nematodes Diatraea saccharalis (sugarcane borer); Spodoptera frugiperda (fall armyworm); Helicoverpa zea (corn earworm); Colaspis brunnea (grape colaspis); Lissorhoptrus oryzophilus (rice water weevil); Sitophilus oryzae (rice weevil); Nephotettix nigropictus (rice leafhopper); Blissus leucopterus leucopterus (chinch bug); Acrosternum hilare (green stink bug).

In rape, the plants are resistant to the pathogenic insects/nematodes Brevicoryne brassicae (cabbage aphid); Phyllotreta cruciferae (Flea beetle); Mamestra configurata (Bertha armyworm); Plutella xylostella (Diamond-back moth); Delia ssp. (Root maggots).

Particularly preferably, the term “plant pathogen” comprises pathogens selected from the group consisting of Blumeria graminis f. sp. hordei, tritici, avenae, secalis, lycopersici, vitis, cucumis, cucurbitae, pisi, pruni, solani, rosae, fragariae, rhododendri, mali, and nicotianae as well as Septoria tritici and Puccinia triticina.

Within the meaning of the present invention a “receptor-like protein kinase” is a protein having an extracellular domain, a transmembrane domain and an intracellular kinase domain which protein catalyzes the transfer of phosphate to a substrate protein. The receptor-like kinase of the present invention has an amino acid sequence selected from the group consisting of:

(a) an amino acid sequence comprising the sequence according to any of SEQ ID Nos. 6, 10, 14, 18, 22 and 25; and
(b) an amino acid sequence which is at least 60% identical to the amino acid sequence according to any of SEQ ID Nos. 6, 10, 14, 18, 22 and 25.

Preferably, the receptor-like kinase of the present invention is encoded by a nucleic acid sequence selected from the group consisting of:

(a) a nucleic acid sequence comprising the sequence according to any of SEQ ID Nos. 1-5, 7-9, 11-13, 15-17, 19-21, 23 and 24 or a fragment of any of these sequences;
(b) a nucleic acid sequence encoding a protein comprising the amino acid sequence according to any of SEQ ID Nos. 1-5, 7-9, 11-13, 15-17, 19-21, 23 and 24;
(c) a nucleic acid sequence comprising a sequence which is at least 70% identical to the sequence according to any of SEQ ID Nos. 1-5, 7-9, 11-13, 15-17, 19-21, 23 and 24 or a fragment of any of these sequences; and
(d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences.

The protein with the amino acid sequence according to SEQ ID No. 6 is encoded by a nucleic acid sequence according to any of SEQ ID NOs. 1-5, preferably by the nucleic acid sequence according to SEQ ID No. 1 or 3. The protein with the amino acid sequence according to SEQ ID No. 10 is encoded by a nucleic acid sequence according to any of SEQ ID NOs. 7-9, preferably by the nucleic acid sequence according to SEQ ID No. 7 or 8. The protein with the amino acid sequence according to SEQ ID No. 14 is encoded by a nucleic acid sequence according to any of SEQ ID NOs. 11-13, preferably by the nucleic acid sequence according to SEQ ID No. 11 or 12. The protein with the amino acid sequence according to SEQ ID No. 18 is encoded by a nucleic acid sequence according to any of SEQ ID NOs. 15-17, preferably by the nucleic acid sequence according to SEQ ID No. 15 or 16. The protein with the amino acid sequence according to SEQ ID No. 22 is encoded by a nucleic acid sequence according to any of SEQ ID NOs. 19-21, preferably by the nucleic acid sequence according to SEQ ID No. 19 or 20. The protein with the amino acid sequence according to SEQ ID No. 25 is encoded by a nucleic acid sequence according to any of SEQ ID NOs. 23 and 24, preferably by the nucleic acid sequence according to SEQ ID No. 23.

The content of a protein within a plant cell is usually determined by the expression level of the protein. Hence, in most cases the terms “content” and “expression” may be used interchangeably. The content of a protein within a cell can be influenced on the level of transcription and/or the level of translation.

The person skilled in the art knows that the activity of a protein is not only influenced by the expression level, but also by other mechanisms such as post-translational modifications such as phosphorylations and acetylations or the interaction with other proteins. The present invention also encompasses methods of increasing the activity of the receptor-like protein kinase which do not affect the content of this protein, such as the expression of a protein which modifies the receptor-like protein kinase by, e.g., phosphorylation, and thereby increases its activity.

The expression level of the nucleic acid coding for the receptor-like protein kinase may be determined in the control plants as well as in the transgenic plants, for example, by RT-PCR analysis or Northern Blot analysis with specific primers or probes. A person skilled in the art knows how to select said probes or primers in order to examine the expression of said nucleic acid. The expression of the protein can also be quantified by determining the strength of the signal in the Northern Blot analysis or by performing a quantitative PCR. Preferably, the expression of the nucleic acid coding for the receptor-like protein kinase is statistically significantly increased by at least the factor 2, 3 or 4, preferably by at least the factor 5, 7 or 10, more preferably by at least the factor 12, 15 or 18, even more preferably by at least the factor 20, 22 or 25 and most preferably by at least the factor 30, 35, 40, 45 or 50. The expression level of the receptor-like protein kinase protein may also be determined by Western Blot analysis using suitable antibodies. Preferably, the amount of the receptor-like protein kinase protein is statistically significantly increased by at least the factor 2, 3 or 4, preferably by at least the factor 5, 7 or 10, more preferably by at least the factor 12, 15 or 18, even more preferably by at least the factor 20, 22 or 25 and most preferably by at least the factor 30, 35, 40, 45 or 50.

The activity of the receptor-like protein kinase may be determined by isolating the receptor-like protein kinase protein from a cell containing it, e.g. by immuno-precipitation, and incubating the protein with a target protein which is phosphorylated by the receptor-like protein kinase and radiolabelled ATP. Then, a sample of the reaction is separated on an SDS-PAGE gel, dried and examined by autoradiography. If the kinase is active, the target protein was phosphorylated and the radiogram will show a corresponding signal which can be quantified and compared to the signal in the control plant.

The increased activity of the receptor-like protein kinase will lead to an increase in target protein phosphorylation by at least the factor 1.5 or 2, preferably by at least the factor 3 or 4, more preferably by at least the factor 5 or 6, even more preferably by at least the factor 7 or 8 and most preferably by at least the factor 9 or 10.

The person skilled in the art is familiar with methods for increasing the content of a given protein. Typically, the method involves introducing into a plant or plant cell a vector which comprises:
(i) a promoter functional in plant cells,
(ii) operatively linked thereto at least one nucleic acid sequence encoding the receptor-like protein kinase as defined herein, and
(iii) optionally, a termination sequence.

According to the present invention, increasing the content and/or the activity of a receptor-like protein kinase is also understood to denote the manipulation of the expression of the endogenous receptor-like protein kinase inherent to the plant/s. This can, for example, be achieved by altering the promoter DNA sequence of a nucleic acid sequence coding for the receptor-like protein kinase. Such a modification, which leads to an increased expression rate of at least one endogenous receptor-like protein kinase, can be effected by deleting or inserting DNA sequences in the promoter region.

Furthermore, an increased expression of at least one endogenous receptor-like protein kinase can be achieved by means of a regulator protein, which is not present in the control plant and which interacts with the promoter of the gene encoding the endogenous receptor-like protein kinase. Such a regulator can be a chimeric protein, which consists of a DNA binding domain and a transcription activator domain, as is described, for example, in WO 96/06166.

A further possibility for increasing the activity and/or the content of the endogenous receptor-like protein kinase is to upregulate transcription factors, which are involved in the transcription of the endogenous genes coding for the receptor-like protein kinase, for example by overexpression. The measures for overexpressing transcription factors are known to the person skilled in the art and within the scope of the present invention are also disclosed for the receptor-like protein kinase.

An alteration of the activity of the endogenous receptor-like protein kinase can also be achieved by influencing the post-translational modifications of the receptor-like protein kinase protein. This can, for example, be done by regulating the activity of enzymes like kinases or phosphatases, which are involved in the post-translational modification of the receptor-like protein kinase, by means of corresponding measures like overexpression or gene silencing.

The expression of the endogenous receptor-like protein kinase can also be regulated via the expression of aptamers specifically binding to the promoter sequences of the receptor-like protein kinase. If the aptamers bind to stimulating promoter regions, the amount and thus, in this case, the activity of the endogenous receptor-like protein kinase is increased.

The skilled person also knows other methods for increasing the content and/or activity of a protein, such as the receptor-like protein kinase encoded by the nucleic acid sequence according to any of SEQ ID Nos. 1-5, 7-9, 11-13, 15-17, 19-21, 23 and 24. For example, a nucleic acid sequence for increasing the content and/or the activity of a protein may be integrated into the natural locus of the sequence by targeted homologous recombination. Such methods are for example described in WO 00/46386 A3, WO 01/89283A1, WO 02/077246 A2 and WO 2007/135022 A1. A method for introducing a targeting sequence differing from the target sequence by 0.1 to 10% by homeologous recombination is described for example in WO 2006/134496 A2.

To cleave DNA sequences within the genomic DNA for introducing a nucleic acid sequence for increasing the content and/or the activity of a protein different enzymes such as meganucleases (WO 2009/114321 A2), zink finger nucleases (WO 2009/042164 A1), transcription activator-like effector nucleases (WO 2011/072246 A2) and chimeric nucleases which comprise a DNA binding domain targeting the nuclease to a specific sequence within the genome (WO 2009/130695 A2) may be used. Such sequence-specific nucleases may also be used to cut the sequence of interest, thereby introducing one or more mutations into said sequence.

Within the scope of the present invention, the method for producing mutant plants, plant cells or plant parts having an increased resistance to pathogens is preferably the TILLING (Targeting Induced Local Lesions IN Genomes) method. In a first step of this method, plant material is mutagenized to introduce at least one mutation into the genome of the plant material. This mutagenesis may be chemical mutagenesis, for example with ethyl methane sulfonate (EMS), mutagenesis by irradiation such as ionizing irradiation or mutagenesis by using sequence-specific nucleases. Single base mutations or point mutations lead to the formation of heteroduplexes which are then cleaved by single strand nucleases such as Cell at the 3′ side of the mutation. The precise position of the at least one mutation within the nucleic acid sequence according to any of SEQ ID Nos. 1-5, 7-9, 11-13, 15-17, 19-21, 23 and 24 can then be determined by denaturing gel electrophoresis or the LICOR gel based system (see, e.g., McCallum et al. (2000) Plant Physiol. 123(2): 439-442; Uauy et al. (2009) BMC Plant Biol. 9:115). If necessary, it can then be determined whether the mutant plant having the at least one point mutation within the nucleic acid sequence according to any of SEQ ID Nos. 1-5, 7-9, 11-13, 15-17, 19-21, 23 and 24 has an increased resistance to pathogens and optionally, suitable plants can be selected.

In the method, the expression construct, the vector and the transgenic plant of the present invention a nucleic acid sequence is used which is selected from the group consisting of:

(a) a nucleic acid sequence comprising the sequence according to any of SEQ ID Nos. 1-5, 7-9, 11-13, 15-17, 19-21, 23 and 24 or a fragment of any of these sequences;
(b) a nucleic acid sequence encoding a protein comprising the amino acid sequence according to any of SEQ ID Nos. 6, 10, 14, 18, 22 and 25;
(c) a nucleic acid sequence comprising a sequence which is at least 70% identical to the sequence according to any of SEQ ID Nos. 1-5, 7-9, 11-13, 15-17, 19-21, 23 and 24 or a fragment of any of these sequences; and
(d) a nucleic acid sequence hybridizing under stringent conditions with a nucleic acid sequence according to any of SEQ ID Nos. 1-5, 7-9, 11-13, 15-17, 19-21, 23 and 24 or a fragment of any of these sequences.

Preferably, a nucleic acid sequence selected from the group consisting of SEQ ID Nos. 1, 3, 7, 8, 11, 12, 15, 16, 19, 20 and 23 is used.

A “fragment” of the nucleic acid sequence according to any of SEQ ID Nos. 1-5, 7-9, 11-13, 15-17, 19-21, 23 and 24 is understood to refer to a smaller part of this nucleic acid sequence which consists of a contiguous nucleotide sequence found in SEQ ID Nos. 1-5, 7-9, 11-13, 15-17, 19-21 and 23-25 and which encodes a protein having the activity of a receptor-like protein kinase.

In case the fragment is described to be a fragment of a sequence with a certain degree of sequence identity to a particular sequence, the fragment shall be a fragment of the sequence which has a certain degree of sequence identity to the particular sequence. Thus, for instance, in expressions like “a nucleic acid sequence comprising a sequence which is at least 70% identical to the sequence according to SEQ ID No. 1 or a fragment of this sequence” the “fragment” in the second alternative refers to a fragment of the sequence which sequence is at least 70% identical to the sequence according to SEQ ID No. 1.

The fragment of any of SEQ ID Nos. 1 and 2 has a length of at least 1000 or 1300 nucleotides, preferably of at least 1500, 1800 or 2000 nucleotides, more preferably of at least 2300, 2600 and 2900 nucleotides and most preferably of at least 3100, 3200 or 3300 nucleotides. The fragment of any of SEQ ID Nos. 4, 5, 17 and 24 has a length of at least 3000 nucleotides, preferably of at least 3500 or 3800 nucleotides, more preferably of at least 4000, 4300 or 4600 nucleotides and most preferably of at least 4700, 4800 or 5000 nucleotides. The fragment of any of SEQ ID Nos. 9 and 13 has a length of at least 12000, 12500, 13000 or 13500 nucleotides, preferably of at least 14000, 14500, 15000, 15500 or 16000 nucleotides, more preferably of at least 16200, 16400, 16600, 16800 or 17000 nucleotides and most preferably of at least 17200, 17400, 17600, 17800, 18000, 18200, 18400 or 18600 nucleotides. The fragment of any of SEQ ID Nos. 7, 15 and 19 has a length of at least 1000, 1100, 1200, 1300, 1400 or 1500 nucleotides, preferably of at least 1600, 1700 or 1800 nucleotides, more preferably of at least 1850, 1900 or 1950 nucleotides and most preferably of at least 2000, 2050, 2080 or 2100 nucleotides. The fragment of any of SEQ ID Nos. 8, 16 and 20 has a length of at least 800, 850, 900 or 950 nucleotides, preferably of at least 1000, 1050, 1100, 1150, 1200, 1250 or 1300 nucleotides, more preferably of at least 1350, 1400, 1450, 1500 or 1550 nucleotides and most preferably of at least 1600, 1650, 1700 or 1750 nucleotides. The fragment of any of SEQ ID Nos. 3 and 11 has a length of at least 1200, 1300, 1350, 1400, 1450 or 1500 nucleotides, preferably of at least 1550, 1600, 1650, 1700, 1750 or 1800 nucleotides, more preferably of at least 1850, 1900, 1950, 2000, 2050, 2100, 2150 or 2200 nucleotides and most preferably of at least 2250, 2300, 2350, 2400, 2450 or 2500 nucleotides. The fragment of SEQ ID No. 12 has a length of at least 700, 750, 800, 850, 900 or 950 nucleotides, preferably of at least 1000, 1050, 1100, 1150, 1200 or 1250 nucleotides, more preferably of at least 1300, 1320, 1340, 1360, 1380, 1400, 1420 or 1440 nucleotides and most preferably of at least 1460, 1480 or 1500 nucleotides. The fragment of SEQ ID No. 21 has a length of at least 5000, 5500, 6000, 6500, 7000 or 7500 nucleotides, preferably of at least 8000, 8200, 8400, 8600, 8800 or 9000 nucleotides, more preferably of at least 9200, 9400, 9600 or 9800 nucleotides and most preferably of at least 10000, 10100, 10200 or 10300 nucleotides. The fragment of SEQ ID No. 23 has a length of at least 500, 550, 600, 650 or 700 nucleotides, preferably of at least 720, 740, 760, 780, 800, 820, 840, 860 or 880 nucleotides, more preferably of at least 900, 910, 920, 930, 940 or 950 nucleotides and most preferably of at least 960, 970, 980, 990, 1000, 1010 or 1020 nucleotides.

The present invention further relates to the use of nucleic acid sequences which are at least 70%, 75% or 80% identical, preferably at least 81, 82, 83, 84, 85 or 86% identical, more preferably at least 87, 88, 89 or 90% identical, even more preferably at least 91, 92, 93, 94 or 95% identical and most preferably at least 96, 97, 98, 99 or 100% identical to the complete sequence according to any of SEQ ID Nos. 1-5, 7-9, 11-13, 15-17, 19-21, 23 and 24 or a fragment of any of these sequences and which encode a protein having the activity of a receptor-like protein kinase.

Within the meaning of the present invention, “sequence identity” denotes the degree of conformity with regard to the 5′-3′ sequence within a nucleic acid molecule in comparison to another nucleic acid molecule. Preferably, the “percentage of sequence identity” is calculated by comparing two optimally aligned sequences over a particular region, determining the number of positions at which the identical base or amino acid is present in both sequences in order to yield the number of matched positions, dividing the number of those matched positions by the total number of positions in the segment being compared and multiplying the result by 100. The sequence identity may be determined using a series of programs, which are based on various algorithms, such as BLASTN, ScanProsite, the laser gene software, etc. As an alternative, the BLAST program package of the National Center for Biotechnology Information (http://www.ncbi.nlm.nih.gov/) may be used applying the default parameters. Here, in addition, the program Sequencher (Gene Codes Corp., Ann Arbor, Mich., USA) using the “dirtydata”-algorithm for sequence comparisons was employed.

The sequence identity refers to the degree of the sequence identity over a length of 1000 or 1300 nucleotides, preferably of 1500, 1800 or 2000 nucleotides, more preferably of 2300, 2600, 2900, 3100, 3200 or 3300 nucleotides and most preferably the whole length of any of SEQ ID Nos. 1 and 2. The sequence identity refers to the degree of the sequence identity over a length of 3000 nucleotides, preferably of 3500 or 3800 nucleotides, more preferably of 4000, 4300, 4600, 4700, 4800 or 5000 nucleotides and most preferably over the whole length of any of SEQ ID Nos. 4, 5, 17 and 24. The sequence identity refers to the degree of the sequence identity over a length of 12000, 12500, 13000 or 13500 nucleotides, preferably of 14000, 14500, 15000, 15500 or 16000 nucleotides, more preferably of 16200, 16400, 16600, 16800, 17000, 17200, 17400, 17600, 17800, 18000, 18200, 18400 or 18600 nucleotides and most preferably the whole length of any of SEQ ID Nos. 9 and 13. The sequence identity refers to the degree of the sequence identity over a length of 1000, 1100, 1200, 1300, 1400 or 1500 nucleotides, preferably of 1600, 1700 or 1800 nucleotides, more preferably of 1850, 1900, 1950, 2000, 2050, 2080 or 2100 nucleotides and most preferably the whole length of any of SEQ ID Nos. 7, 15 and 19. The sequence identity refers to the degree of the sequence identity over a length of 800, 850, 900 or 950 nucleotides, preferably of 1000, 1050, 1100, 1150, 1200, 1250 or 1300 nucleotides, more preferably of 1350, 1400, 1450, 1500, 1550, 1600, 1650, 1700 or 1750 nucleotides and most preferably the whole length of any of SEQ ID Nos. 8, 16 and 20. The sequence identity refers to the degree of the sequence identity over a length of 1200, 1300, 1350, 1400, 1450 or 1500 nucleotides, preferably of 1550, 1600, 1650, 1700, 1750 or 1800 nucleotides, more preferably of 1850, 1900, 1950, 2000, 2050, 2100, 2150, 2200, 2250, 2300, 2350, 2400, 2450 or 2500 nucleotides and most preferably the whole length of any of SEQ ID Nos. 3 and 11. The sequence identity refers to the degree of the sequence identity over a length of 700, 750, 800, 850, 900 or 950 nucleotides, preferably of 1000, 1050, 1100, 1150, 1200 or 1250 nucleotides, more preferably of 1300, 1320, 1340, 1360, 1380, 1400, 1420, 1440, 1460, 1480 or 1500 nucleotides and most preferably the whole length of SEQ ID No. 12. The sequence identity refers to the degree of the sequence identity over a length of 5000, 5500, 6000, 6500, 7000 or 7500 nucleotides, preferably of 8000, 8200, 8400, 8600, 8800 or 9000 nucleotides, more preferably of 9200, 9400, 9600, 9800, 10000, 10100, 10200 or 10300 nucleotides and most preferably the whole length of SEQ ID No. 21. The sequence identity refers to the degree of the sequence identity over a length of 500, 550, 600, 650 or 700 nucleotides, preferably of 750, 800, 850 or 900 nucleotides, more preferably of 920, 940, 960, 980, 1000, 1010, 1020 or 1030 nucleotides and most preferably the whole length of SEQ ID No.

23.

The present invention further relates to the use of nucleic acid sequences which hybridize under stringent conditions with a nucleic acid sequence according to any of SEQ ID Nos. 1-5, 7-9, 11-13, 15-17, 19-21, 23 and 24 or a fragment of any of these sequences and which encode an amino acid sequence having the activity of a receptor-like protein kinase.

The term “hybridizing under stringent conditions” denotes in the context of the present invention that the hybridization is implemented in vitro under conditions which are stringent enough to ensure a specific hybridization. Stringent in vitro hybridization conditions are known to those skilled in the art and may be taken from the literature (e.g. Sambrook and Russell (2001) Molecular Cloning: A Laboratory Manual, 3rd edition, Cold Spring Harbour Laboratory Press, Cold Spring Harbour, N.Y.). The term “specific hybridization” refers to the circumstance that a molecule, under stringent conditions, preferably binds to a certain nucleic acid sequence, i.e. the target sequence, if the same is part of a complex mixture of, e.g. DNA or RNA molecules, but does not, or at least very rarely, bind to other sequences.

Stringent conditions depend on the circumstances. Longer sequences hybridize specifically at higher temperatures. In general, stringent conditions are chosen such that the hybridization temperature is about 5° C. below the melting point (Tₘ) of the specific sequence at a defined ionic strength and at a defined pH value. Tₘ is the temperature (at a defined pH value, a defined ionic strength and a defined nucleic acid concentration), at which 50% of the molecules complementary to the target sequence hybridize to the target sequence in the state of equilibrium. Typically, stringent conditions are conditions, where the salt concentration has a sodium ion concentration (or concentration of a different salt) of at least about 0.01 to 1.0 M at a pH value between 7.0 and 8.3, and the temperature is at least 30° C. for small molecules (i.e. 10 to 50 nucleotides, for example). In addition, stringent conditions may include the addition of substances, such as, e.g., formamide, which destabilise the hybrids. At hybridization under stringent conditions, as used herein, normally nucleotide sequences which are at least 60% homologous to each other hybridize to each other. Preferably, said stringent conditions are chosen such that sequences which are about 65%, preferably at least about 70%, and especially preferably at least about 75% or higher homologous to each other, normally remain hybridized to each other. A preferred but non-limiting example of stringent hybridization conditions is hybridizations in 6× sodium chloride/sodium citrate (SSC) at about 45° C., followed by one or more washing steps in 0.2×SSC, 0.1% SDS at 50 to 65° C. The temperature depends on the type of the nucleic acid and is between 42° C. and 58° C. in an aqueous buffer having a concentration of 0.1 to 5×SSC (pH value 7.2).

If an organic solvent, e.g. 50% formamide, is present in the above-mentioned buffer, the temperature is about 42° C. under standard conditions. Preferably, the hybridisation conditions for DNA:DNA hybrids are, for example, 0.1×SSC and 20° C. to 45° C., preferably 30° C. to 45° C. Preferably, the hybridisation conditions for DNA:RNA hybrids are, for example, 0.1×SSC and 30° C. to 55° C., preferably between 45° C. and 55° C. The above-mentioned hybridization temperatures are determined, for example, for a nucleic acid which is 100 base pairs long and has a G/C content of 50% in the absence of formamide. Those skilled in the art know how to determine the required hybridization conditions using text books such as those mentioned above or the following textbooks: Current Protocols in Molecular Biology, John Wiley & Sons, N.Y. (1989), Hames and Higgins (publ.) 1985, Nucleic Acids Hybridization: A Practical Approach, IRL Press at Oxford University Press, Oxford; Brown (publ.) 1991, Essential Molecular Biology: A Practical Approach, IRL Press at Oxford University Press, Oxford.

Typical hybridization and washing buffers for example have the following composition:

Pre-hybridization solution: 0.5% SDS
5×SSC
50 mM NaPO₄, pH 6.8
0.1% sodium pyrophosphate
5×Denhardt's solution
100 μg/mL salmon sperm DNA
   Hybridization solution: pre-hybridization solution
1×10⁶ cpm/mL probe (5-10 min 95° C.)

### 20×SSC: 3 M NaCl

0.3 M sodium citrate
ad pH 7 with HCl
   50×Denhardt's reagent: 5 g Ficoll
5 g polyvinylpyrrolidone
5 g bovine serum albumin
ad 500 mL aqua destillata

A typical procedure for hybridization is as follows:

Optional: wash blot 30 min in 1×SSC/0.1% SDS at 65° C.
Pre-hybridization: at least 2 h at 50-55° C.
Hybridization: over night at 55-60° C.

| | | | |
|---|---|---|---|
| Washing: | 05 min | 2x SSC/0.1% SDS | hybridization temp. |
| | 30 min | 2x SSC/0.1% SDS | hybridization temp. |
| | 30 min | 1x SSC/0.1% SDS | hybridization temp. |
| | 45 min | 0.2x SSC/0.1% SDS | 65° C. |
| | 5 min | 0.1x SSC | room temperature |

Those skilled in the art know that the given solutions and the presented protocol may be modified or have to be modified, depending on the application.

The nucleic acid sequence hybridizing to a fragment of the sequence according to any of SEQ ID Nos. 1 and 2 under stringent conditions has a length of at least 1000 or 1300 nucleotides, preferably of at least 1500, 1800 or 2000 nucleotides, more preferably of at least 2300, 2600, 2900 nucleotides and most preferably of at least 3100, 3200 or 3300 nucleotides. The nucleic acid sequence hybridizing to a fragment of the sequence according to any of SEQ ID Nos. 4, 5, 17 and 24 under stringent conditions has a length of at least 3000 nucleotides, preferably of at least 3500 or 3800 nucleotides, more preferably of at least 4000, 4300, 4600 nucleotides and most preferably of at least 4700, 4800 or 5000 nucleotides. The nucleic acid sequence hybridizing to a fragment of the sequence according to any of SEQ ID Nos. 9 and 13 under stringent conditions has a length of at least 12000, 12500, 13000 or 13500 nucleotides, preferably of at least 14000, 14500, 15000, 15500 or 16000 nucleotides, more preferably of at least 16200, 16400, 16600, 16800, 17000, 17200, 17400 nucleotides and most preferably of at least 17600, 17800, 18000, 18200, 18400 or 18600 nucleotides. The nucleic acid sequence hybridizing to a fragment of the sequence according to any of SEQ ID Nos. 7, 15 and 19 under stringent conditions has a length of at least 1000, 1100, 1200, 1300, 1400 or 1500 nucleotides, preferably of at least 1600, 1700 or 1800 nucleotides, more preferably of at least 1850, 1900, 1950, 2000 nucleotides and most preferably of at least 2050, 2080 or 2100 nucleotides. The nucleic acid sequence hybridizing to a fragment of the sequence according to any of SEQ ID Nos. 8, 16 and 20 under stringent conditions has a length of at least 800, 850, 900 or 950 nucleotides, preferably of at least 1000, 1050, 1100, 1150, 1200, 1250 or 1300 nucleotides, more preferably of at least 1350, 1400, 1450, 1500 nucleotides and most preferably of at least 1550, 1600, 1650, 1700 or 1750 nucleotides. The nucleic acid sequence hybridizing to a fragment of the sequence according to any of SEQ ID Nos. 3 and 11 under stringent conditions has a length of at least 1200, 1300, 1350, 1400, 1450 or 1500 nucleotides, preferably of at least 1550, 1600, 1650, 1700, 1750 or 1800 nucleotides, more preferably of at least 1850, 1900, 1950, 2000, 2050, 2100 nucleotides and most preferably of at least 2150, 2200, 2250, 2300, 2350, 2400, 2450 or 2500 nucleotides. The nucleic acid sequence hybridizing to a fragment of the sequence according to SEQ ID No. 12 under stringent conditions has a length of at least 700, 750, 800, 850, 900 or 950 nucleotides, preferably of at least 1000, 1050, 1100, 1150, 1200 or 1250 nucleotides, more preferably of at least 1300, 1320, 1340, 1360, 1380, 1400 nucleotides and most preferably of at least 1420, 1440, 1460, 1480 or 1500 nucleotides. The nucleic acid sequence hybridizing to a fragment of the sequence according to SEQ ID No. 21 under stringent conditions has a length of at least 5000, 5500, 6000, 6500, 7000 or 7500 nucleotides, preferably of at least 8000, 8200, 8400, 8600, 8800 or 9000 nucleotides, more preferably of at least 9200, 9400, 9600, 9800 or 10000 nucleotides and most preferably of at least 10100, 10200 or 10300 nucleotides. The nucleic acid sequence hybridizing to a fragment of the sequence according to SEQ ID No. 23 under stringent conditions has a length of at least 500, 550, 600, 650, 700 or 750 nucleotides, preferably of at least 800, 820, 840, 860, 880 or 900 nucleotides, more preferably of at least 920, 940, 960, 980 or1000 nucleotides and most preferably of at least 1010, 1020 or 1030 nucleotides.

In the context of the above, the term “encodes a protein having the activity of a receptor-like protein kinase” means that the encoded protein has essentially the same activity as the receptor-like protein kinase encoded by a nucleic acid sequence of any of SEQ ID Nos. 1-5, 7-9, 11-13, 15-17, 19-21, 23 and 24. “Essentially the same activity” means that the protein has at least 5 or 10%, preferably at least 20, 30 or 40%, more preferably 50, 60 or 70% and most preferably at least 80, 85, 88, 90, 95, or 98% of the activity of the receptor-like protein kinase encoded by a sequence of any of SEQ ID Nos. 1-5, 7-9, 11-13, 15-17, 19-21, 23 and 24. The activity of the receptor-like protein kinase can be determined as described above.

In order to produce the expression constructs or vectors of the present invention, a suitable nucleic acid sequence may for example be inserted into an appropriate expression construct or vector by restriction digestion and subsequent ligation using techniques well-known to the person skilled in the art and described in the textbooks referred to herein.

Within the scope of the present invention, the terms “expression construct” or “expression cassette” mean a nucleic acid molecule which contains all elements which are necessary for the expression of a nucleic acid sequence, i.e. the nucleic acid sequence to be expressed under the control of a suitable promoter and optionally further regulatory sequences such as termination sequences. An expression cassette of the present invention may be part of an expression vector which is transferred into a plant cell or may be integrated into the chromosome of a transgenic plant after transformation.

As used herein, the term “vector” refers to a nucleic acid molecule capable of transporting another nucleic acid to which it has been linked and may be used herein interchangeably with the term “recombinant nucleic acid molecule”. One type of vector is a “plasmid”, which refers to a circular double stranded DNA loop into which additional DNA segments can be ligated. In the present specification, “plasmid” and “vector” can be used interchangeably as the plasmid is the most commonly used form of vector. A vector can be a binary vector or a T-DNA that comprises a left and a right border and may include a gene of interest in between. The term “expression vector” means a vector capable of directing expression of a particular nucleotide sequence in an appropriate host cell. An expression vector comprises a regulatory nucleic acid element operably linked to a nucleic acid of interest, which is—optionally—operably linked to a termination signal and/or other regulatory element.

The term “promoter” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

The promoter used in the present invention may be a constitutive promoter, an inducible promoter or a tissue-specific promoter.

Constitutive promoters, include the 35S CaMV promoter (Franck et al. (1980) Cell 21: 285-294), the ubiquitin promoter (Binet et al. (1991) Plant Science 79: 87-94), the Nos promoter (An et al. (1990) The Plant Cell 3: 225-233), the MAS promoter (Velten et al. (1984) EMBO J. 3: 2723-230), the maize H3 histone promoter (Lepetit et al. (1992) Mol Gen. Genet 231: 276-285), the ALS promoter (WO 96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the Rubisco small subunit promoter (U.S. Pat. No. 4,962,028) and the actin promoter (McElroy et al. (1990) Plant Cell 2: 163-171).

In another embodiment, the promoter is a regulated promoter. A “regulated promoter” refers to a promoter that directs gene expression not constitutively, but in a temporally and/or spatially restricted manner, and includes both tissue-specific and inducible promoters. Different promoters may direct the expression of a polynucleotide or regulatory element in different tissues or cell types, or at different stages of development, or in response to different environmental conditions.

Wound-, light- or pathogen-induced promoters and other development-dependent promoters or control sequences may also be used (Xu et al. (1993) Plant Mol. Biol. 22: 573-588; Logemann et al. (1989) Plant Cell 1: 151-158; Stockhaus et al. (1989) Plant Cell 1: 805-813; Puente et al. (1996) EMBO J. 15: 3732-3734; Gough et al. (1995) Mol. Gen. Genet. 247: 323-337). A summary of useable control sequences may be found, for example, in Zuo et al. (2000) Curr. Opin. Biotech. 11: 146-151.

A “tissue-specific promoter” or “tissue-preferred promoter” refers to a regulated promoter that is not expressed in all plant cells, but only in one or more cell types in specific organs (such as leaves or seeds), specific tissues (such as embryo or cotyledon), or specific cell types (such as leaf parenchyma or seed storage cells).

Suitable tissue-specific promoters include, e.g., epidermis-specific promoters, such as the GSTA1 promoter (Altpeter et al. (2005) Plant Mol Biol. 57: 271-83), or promoters of photosynthetically active tissues, such as the ST-LS1 promoter (Stockhaus et al. (1987) Proc. Natl. Acad. Sci. USA 84: 7943-7947; Stockhaus et al. (1989) EMBO J. 8: 2445-2451). The promoters of phosphoenolpyruvate-carboxylase from corn (Hudspeth et al. (1989) Plant Mol. Biol. 12: 579) or of fructose-1,6-bisphosphatase from potato (WO 98/18940), which impart leaf-specific expression, are also considered to be tissue-specific promoters. Further preferred promoters are those which are in particular active in fruits. Examples of these are the promoter of a polygalacturonase gene, e.g. from tomato, which mediates expression during the ripening process of tomato fruits (Nicholass et al. (1995) Plant Mol. Biol. 28: 423-435), the promoter of an ACC oxidase, e.g. from apples, which mediates ripening and fruit specificity in transgenic tomatoes (Atkinson et al. (1998) Plant Mol. Biol. 38: 449-460), or the 2A11 promoter from tomato (van Haaren et al. (1991) Plant Mol. Biol. 17: 615-630). Further, the chemically inducible Tet repressor system (Gatz et al. (1991) Mol. Gen. Genet. 227: 229-237) may be used.

Other suitable promoters may be taken from the literature, e.g. Ward ((1993) Plant Mol. Biol. 22: 361-366). The same applies to inducible and cell- or tissue-specific promoters, such as meristem-specific promoters which have also been described in the literature and which are suitable within the scope of the present invention as well.

Particularly suitable promoters for the method of the present invention are pathogen-inducible promoters, and especially those, which are induced by pathogenic fungi and not by useful fungi (e.g. mycorrhiza in the soil, such as the GER4 promoter (WO 2006/128882). Further promoters which are inducible by fungi include promoters such as the GAFP-2 promoter (Sa et al. (2003) Plant Cell Rep. 22: 79-84), which, e.g., is induced by the fungus Trichoderma viride, or the PAL promoter which is induced by inoculation with Pyricularia oryzae (Wang et al. (2004) Plant Cell Rep. 22: 513-518).

Also particularly suitable in the method of the present invention are promoters which are active on the site of pathogen entry, such as epidermis-specific promoters. Suitable epidermis-specific promoters include, but are not limited to, the GSTA1 promoter (Accession number X56012), the GLP4 promoter (Wei et al. (1998) Plant Mol. Biol. 36: 101), the GLP2a promoter (Accession number AJ237942), the Prx7 promoter (Kristensen et al. (2001) Mol. Plant Pathol. 2(6): 311), the GerA promoter (Wu et al. (2000) Plant Phys Biochem. 38: 685), the OsROC1 promoter (Accession number AP004656), the RTBV promoter (Kloeti et al. (1999) PMB 40: 249); the chitinase ChtC2 promoter (Ancillo et al. (2003) Planta 217(4): 566), the AtProT3 promoter (Grallath et al. (2005) Plant Physiol. 137(1): 117) and the SHN promoters from Arabidopsis (Aaron et al. (2004) Plant Cell 16(9): 2463).

Furthermore, those skilled in the art are able to isolate further suitable promoters by means of routine procedures.

The skilled person knows that the use of inducible promoters allows for the production of plants and plant cells which only transiently express the sequences of the present invention. Such transient expression allows for the production of plants which show only transiently increased pathogen resistance. Such transiently increased resistance may be desired, if, for example, there is an acute risk of fungal contamination, and therefore the plants only have to be resistant to the fungus for a certain period of time. Further situations, in which transient resistance is desirable, are known to those skilled in the art. The skilled person also knows that transient expression and transient resistance may be achieved using vectors which do not replicate stably in plant cells and which carry the respective sequences for silencing of fungal genes.

In a preferred embodiment of the method of the invention, the actin promoter from Oryza sativa providing constitutive transgene expression is used to express a nucleic acid sequence of the present invention.

The vectors which are used in the method of the present invention may further comprise regulatory elements in addition to the nucleic acid sequence to be transferred. Which specific regulatory elements must be included in said vectors depends on the procedure which is to be used for said vectors. Those skilled in the art, who are familiar with the various methods for producing transgenic plants in which the expression of a protein is inhibited know which regulatory elements and also other elements said vectors must include.

Typically, the regulatory elements which are contained in the vectors ensure the transcription and, if desired, the translation in the plant cell.

The term “transcription regulatory element” as used herein refers to a polynucleotide that is capable of regulating the transcription of an operably linked polynucleotide. It includes, but is not limited to, promoters, enhancers, introns, 5′ UTRs, and 3′ UTRs.

With respect to nucleic acid sequences or DNA sections in expression constructs or vectors the terms “operatively linked” and “operably linked” mean that nucleic acid sequences are linked to each other such that the function of one nucleic acid sequence is influenced by the other nucleic acid sequence. For example, if a nucleic acid sequence is operably linked to a promoter, its expression is influenced by said promoter.

So-called termination sequences are sequences which ensure that the transcription or the translation is terminated properly. If the introduced nucleic acids are to be translated, said nucleic acids are typically stop codons and corresponding regulatory sequences; if the introduced nucleic acids are only to be transcribed, said nucleic acids are normally poly-A sequences.

The vectors of the present invention may for example also comprise enhancer elements as regulatory elements, resistance genes, replication signals and further DNA regions which allow for a propagation of the vectors in bacteria, such as E. coli. Regulatory elements also comprise sequences which lead to a stabilization of the vectors in the host cells. In particular, such regulatory elements comprise sequences which enable a stable integration of said vector in the host genome of the plant or autonomous replication of said vector in the plant cells. Such regulatory elements are known to those skilled in the art.

A number of well-known techniques are available for introducing DNA into a plant host cell, and those skilled in the art may easily determine the suitable technique for each case. Said techniques comprise the transformation of plant cells with T-DNA using Agrobacterium tumefaciens or Agrobacterium rhizogenes as transformation means, viral infection by using viral vectors (EP 0 067 553; U.S. Pat. No. 4,407,956, WO 95/34668; WO 93/03161), the fusion of protoplasts, polyethylene glycol-induced DNA uptake, liposome-mediated transformation (U.S. Pat. No. 4,536,475), incubation of dry embryos in DNA-comprising solution, microinjection, the direct gene transfer of isolated DNA in protoplasts, the electroporation of DNA, the introduction of DNA by the biolistic procedure, as well as other possibilities. Thereby, stable as well as transient transformants may be produced.

For injection and electroporation of DNA in plant cells, the used plasmids do not need to fulfil special requirements per se. The same applies to direct gene transfer. Simple plasmids, such as pUC derivatives, may be used. If, however, whole plants are to be regenerated from cells which were transformed in such manner, the presence of a selectable marker gene may become necessary. Those skilled in the art know all commonly used selection markers, and thus there is no difficulty to select a suitable marker. Common selection markers create resistance in the transformed plant cells to a biocide or antibiotic, such as kanamycin, G418, bleomycin, hygromycin, methotrexate, glyphosate, streptomycin, sulfonyl urea, gentamycin or phosphinotricin and the like or may confer tolerance to D-amino acids such as D-alanine. However, it is also possible to select transformed cells by PCR, i.e. without the use of selection markers.

Depending on the introduction method of the desired genes into the plant cell, further DNA sequences may become necessary. If, for example, the Ti or Ri plasmid is used for the transformation of the plant cell, at least the right border, or very often both the right and the left border of the T-DNA contained in the Ti and Ri plasmid needs to be linked to the genes to be inserted.

If agrobacteria are used for the transformation, the DNA to be inserted needs to be cloned into special plasmids, i.e. either into an intermediate vector or into a binary vector. The intermediate vectors may be integrated into the Ti or Ri plasmid of the agrobacteria by means of homologous recombination due to sequences which are homologous to sequences in the T-DNA, which contains the vir region required for the transfer of the T-DNA. Intermediate vectors are not able to replicate in agrobacteria. By means of a helper plasmid, the intermediate vector may be transferred to Agrobacterium tumefaciens (conjugation). Binary vectors are able to replicate in both E. coli and in agrobacteria. Said vectors contain a selection marker gene and a linker or polylinker located between the right and left T-DNA border region. The vector may be transformed directly into the agrobacteria (Holsters et al. (1978) Molecular and General Genetics 163: 181-187). The agrobacterium, serving as host cell, is to contain a plasmid which includes a vir region. The vir region is necessary for the transfer of the T-DNA into the plant cell. In addition, T-DNA may be present. The agrobacterium transformed in such a manner is used for the transformation of plant cells.

For the transfer of the DNA into the plant cell, plant explants may be cultivated with Agrobacterium tumefaciens or Agrobacterium rhizogenes. From the infected plant material (e.g. leaf cuttings, stem sections, roots, but also protoplasts or suspension-cultivated plant cells) whole plants may be regenerated in a suitable medium which may contain antibiotics, biocides or D-amino acids for the selection of transformed cells, if a selection marker was used in the transformation. The regeneration of the plants is performed according to standard regeneration procedures using well-known culture media. The plants or plant cells obtained this way may then be examined for the presence of the introduced DNA.

Other possibilities for introducing foreign DNA using the biolistic method or by protoplast transformation are well-known to those skilled in the art (see L. Willmitzer (1993) Transgenic Plants in: Biotechnology, A Multi-Volume Comprehensive Treatise (publisher: H. J. Rehm et al.), volume 2, 627-659, VCH Weinheim, Germany).

Monocotyledonous plants or the cells thereof may also be transformed using vectors which are based on agrobacteria (see e.g. Chan et al. (1993) Plant Mol. Biol. 22: 491-506). Alternative systems for the transformation of monocotyledonous plants or the cells thereof are transformation by the biolistic approach (Wan and Lemaux (1994) Plant Physiol. 104: 37-48; Vasil et al. (1993) Bio/Technology 11: 1553-1558; Ritala et al. (1994) Plant Mol. Biol. 24: 317-325; Spencer et al. (1990) Theor. Appl. Genet. 79: 625-631), the protoplast transformation, the electroporation of partially permeabilized cells, and the insertion of DNA by means of glass fibres.

The vectors described herein can be directly transformed into the plastid genome. Plastid expression, in which genes are inserted by homologous recombination into the several thousand copies of the circular plastid genome present in each plant cell, takes advantage of the enormous copy number over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. No. 5,451,513; U.S. Pat. No. 5,545,817; U.S. Pat. No. 5,545,818 and U.S. Pat. No. 5,877,462, in WO 95/16783 and WO 97/32977, and in McBride et al. (1994) Proc. Natl. Acad. Sci. USA 91: 7301-7305.

The transformed cells grow within the plant in the usual manner (see also McCormick et al. (1986) Plant Cell Reports 5: 81-84). The resulting plants may be cultivated in the usual manner, and may be crossed with plants which have the same transformed genes or other genes. The hybrid individuals resulting therefrom have the respective phenotypical properties.

The method of the present invention may further comprise the step of crossing the transgenic plant produced by the method of the present invention with another plant in which the content and/or the activity of the receptor-like protein kinase is not increased and selecting transgenic progeny in which the content and/or the activity of the receptor-like protein kinase is increased. The other plant in which the content and/or the activity of the receptor-like protein kinase is not increased is preferably from the same species as the transgenic plant and may be a wild-type plant, i.e. a plant which does not contain any transgenic nucleic acid sequence, or it may be a transgenic plant which contains a transgenic nucleic acid sequence other than the nucleic acid sequences disclosed herein, e.g. a transgenic nucleic acid sequence coding for another protein involved in pathogen resistance or a protein conferring resistance to abiotic stress. The other plant is preferably an elite variety which is characterized by at least one favourable agronomic property which is stably present in said elite variety. Methods for determining whether the content and/or activity of the receptor-like protein kinase is increased are discussed above. An “elite variety” within the meaning of the present invention is a variety which is adapted to specific environmental conditions and/or which displays at least one superior characteristic such as an increased yield compared to non-elite varieties.

The transgenic progeny of the above crossing step can be further crossed with each other to produce true breeding lines. For this purpose the transgenic progeny of the above cross in which the content and/or the activity of the receptor-like protein kinase is increased is inbred and the transgenic progeny of this crossing step is selected and again inbred. This inbreeding step is repeated until a true breeding line is established, for example at least five times, six times or seven times. A “true breeding plant” or “inbred plant” is a plant which upon self-pollination produces only offspring which is identical to the parent with respect to at least one trait, in the present case the transgene which increases the content and/or the activity of the receptor-like protein kinase.

The true breeding lines can then be used in hybrid breeding yielding F1 hybrids which can be marketed. This method is particularly suitable for example for maize and rice plants.

Alternatively, the true breeding lines can be further inbred in a linebreeding process. This method is particularly suitable for example for wheat and barley plants.

According to common procedures, transgenic lines which are homozygous for the introduced nucleic acid molecules may also be identified and examined with respect to pathogen resistance compared to the pathogen resistance of hemizygous lines.

Of course, plant cells which contain the expression constructs, vectors or recombinant nucleic acid molecules of the present invention may also be further cultivated as plant cells (including protoplasts, calli, suspension cultures and the like).

The method of the present invention may additionally comprise the reduction of the content and/or the activity of at least one, for example two or three, plant proteins which mediate pathogen susceptibility. Suitable genes include the Mlo gene (WO 00/01722), the Bax inhibitor-1 gene (Eichmann et al. (2010) Mol. Plant Microbe Interact. 23(9): 1217-1227) and the Pmr genes (Vogel and Somerville (2000) Proc. Natl. Acad. Sci. USA 97(4): 1897-1902).

The transgenic plants of the present invention or parts thereof can be used as fodder plants or for producing feed. Fodder is intended to mean any agricultural foodstuff which is specifically used to feed domesticated animals such as cattle, goats, sheep and horses. It includes includes hay, straw, silage and also sprouted grains and legumes. The person skilled in the art knows that it may be necessary to treat the transgenic plants of the present invention to make them suitable for use as fodder. The term feed is intended to mean a dry feed which can be blended from various raw materials and additives such as soybean shred or barley shred in a feed mill.

The transgenic or mutant seed of the transgenic or mutant plants of the present invention can be used to prepare flour, in particular if the transgenic or mutant plants are monocotyledonous plants such as barley or wheat.

Hence, another embodiment of the present invention is a method for the production of a product comprising the steps of:
(a) growing the plants of the present invention or plants obtainable by the methods of the present invention; and
(b) producing said product from or by the plants of the invention and/or parts, e.g. seeds of these plants.

In a further embodiment the method comprises the steps of:
(a) growing the plants of the present invention or plants obtainable by the methods of the present invention;
(b) removing the harvestable parts from the plants and
(c) producing said product from or by the harvestable parts of the plants of the invention.

In one embodiment the product produced by said methods of the invention is flour comprising the nucleic acid sequence which increases the content and/or activity of a receptor-like protein kinase.

The flour prepared from the transgenic seed of the present invention can be distinguished from the flour prepared from other plants by the presence of the transgenic nucleic acid sequence, the expression construct or the vector of the present invention. For example, if the transgenic nucleic acid sequence is expressed under the control of a promoter which is not endogenous to the transgenic plant, the presence of the promoter can be detected in the flour prepared from the transgenic seed.

The flour prepared from the mutant seed of the present invention can be distinguished from the flour prepared from other plants by the presence of the at least one point mutation within the nucleic acid sequence defined herein.

Harvestable parts of the transgenic plants of the present invention are also a subject of the invention. Preferably, the harvestable parts comprise a nucleic acid sequence which increases the content and/or activity of a receptor-like protein kinase, i.e. this nucleic acid sequence is detectable in the harvestable parts by conventional means. The harvestable plants may be seeds, roots, leaves, stems, and/or flowers comprising the nucleic acid sequence which increases the content of a receptor-like protein kinase. Preferred harvestable parts are seeds comprising the nucleic acid sequence which increases the content of a receptor-like protein kinase.

The identification of receptor-like protein kinases as proteins involved in pathogen resistance and the use thereof for producing transgenic plants with increased pathogen resistance will be described in the following. The following examples shall not limit the scope of the present invention. The content of all literature references, patent applications, patent specifications and patent publications, which are cited in this patent application, is incorporated herein by reference.

### Examples

### 1. Transformation of Wheat Leaves with BAC Clones

### 1.1. Transformation

BAC clones from a barley BAC library (Yu et al. (2000) TAG 101: 1093-1099) which carry genomic DNA encoding the receptor-like protein kinases of the present invention were transformed into wheat leaves using biolistic transformation with a gene gun (Bio-Rad-model PDS-1000/He, hepta adapter) and the method according to Douchkov et al. (2005) Mol. Plant-Microbe Interact 18: 755-761. The following plasmid mixtures were used for transformation (Table 6):

| Plasmid | transformation 1 | transformation 2 | transformation 3 | transformation 4 | transformation 5 |
|---|---|---|---|---|---|
| pUbiGUS | 7 μg/bombardement | 7 μg/bombardement | 7 μg/bombardement | 7 μg/bombardement | 7 μg/bombardement |
| (reporter gene | | | | | |
| construct) | | | | | |
| pIPKTA9 (empty | 7 μg/bombardement | — | — | — | — |
| vector) | | | | | |
| pJP01 | — | — | 7 μg/bombardement | — | — |
| (TaPrx103; | | | | | |
| positive control) | | | | | |
| BAC 632F23 | — | 14 μg/bombardement | — | — | — |
| (negative | | | | | |
| control) | | | | | |
| Test BAC | — | — | — | 14 μg/bombardement | — |

Per bombardment, 2.18 mg of gold particles, (1.0 mm diameter, particle density 25 mg/mL in 50% of glycerol) were mixed with 14 or 21 μg of supercoiled DNA mixed as described in Table 6 above and then 1 M Ca(NO₃)₂ pH 10 was added so that the final concentration of Ca(NO₃)₂ was 0.5M. The suspension was centrifuged and the pellet was washed with 70% (v/v) ethanol, before the particles were resuspended in 96% (v/v) ethanol and distributed on 7 macrocarriers.

For biolistic transformation vacuum (3.6×10³ Pa) was applied to seven leaf segments of seven days old wheat plants (variety Kanzler) per transformation with a helium pressure wave of 7.6×10⁶ Pa. For transformation the leaf segments were placed on a petri dish with 0.5% phytoagar containing 20 μg/ml benzimidazole. Then the leaves were incubated at 20° C. and indirect daylight for four hours.

### 1.2. Inoculation of Leaf Segments

The bombarded leaves were transferred to large, square Petri dishes containing 1% w/v phytoagar with 20 ppm of benzimidazole. The inoculation with wheat mildew conidia was performed in an inoculation tower by shaking conidia from strongly infected wheat leaves (about 200 conidia/mm²) into the tower. After five minutes the dishes were removed, closed and incubated at 20° C. and indirect daylight for 40 hours.

### 1.3. GUS Staining (for Staining the Transformed Cells)

40 h after inoculation, the leaves were contacted with the GUS detection solution (100 mM sodium phosphate, pH 7.0; 10 mM EDTA; 5 mM K₃[Fe(CN)₆], 5 mM K₄[Fe(CN)₆]; 0.1% Triton X-100; 20% methanol and 1 mg/ml 3-bromo-4-chloro-3-indolyl-β-D-glucuronic acid) under vacuum and incubated in this solution over night at 37° C. After removing the detection solution the leaves were destained with a solution containing 7.5% TCA and 50% (v/v) methanol for 15 minutes at 20° C.

The staining of the cells was detected with a Zeiss Axiolab microscope at a magnifaction of 200. Cells expressing GUS were stained blue. Using quantitative microscopy the number of total GUS-stained cells and the number of GUS-stained cells carrying at least one haustorium of wheat mildew per transformation was determined. The susceptibility index is calculated as the number of haustorium-carrying GUS-positive cells per total number of GUS-positive cells.

Using this approach, the following results were obtained (Table 7):

| | Rel. | p two- | p one- | |
|---|---|---|---|---|
| transformed DNA | SI (%)^{a} | sided^{b} | sided | N^{c} |
| BAC632F23 (negative | 100 | — | — | 10 |
| control)^{d} | | | | |
| pJP01 (positive control)^{e} | 12.6 | 6.292E−07 | 3.146E−07 | 10 |
| BAC PHENOME_WP3_103 | 51.0 | 0.0430 | 0.0215 | 4 |
| comprising SEQ ID Nos. | | | | |
| 7, 8, 19, 20, 23 and 24 | | | | |
| BAC PHENOME_WP3_107 | 55.2 | 0.0813 | 0.0406 | 5 |
| comprising SEQ ID Nos. 11, | | | | |
| 12, 15 and 16 | | | | |
| BAC PHENOME_WP3_104 | 62.9 | 0.0256 | 0.0128 | 5 |
| comprising SEQ ID Nos. 1-4 | | | | |

^{a}relative susceptibility index in % of negative control
^{b}one-sample t-test relative to the hypothetical value 100
^{c}number of independent bombardements
^{d}sequenced BAC clone from barley which does not contain identified genes
^{e}vector expressing class III peroxidase cDNA sequence TaPrx103 from wheat under the control of the cauliflower mosaic virus 35S promoter and terminator (see Altpeter et al. (2005) Plant. Mol. Biol. 57: 271-283).

This experiment shows that the transient expression of the sequences of the present invention in wheat leaves increases the resistance to Blumeria graminis f.sp. tritici in comparison to leaves transformed with the corresponding empty vector by about 30-50%. Hence, the expression of a receptor-like protein kinase leads to non-host resistance in wheat.

### 2. Transformation of Wheat Leaves with Expression Vectors Encoding the Receptor-Like Protein Kinases (RLKs)

### 2.1. Cloning of an Expression Vector Encoding RLK Comp) 3Hcluster 2

To obtain an expression vector for transient transformation of plant tissue, the RLK_compl_{—}3 Hcluster_{—}2 sequence was amplified using the BAC clone BAC PHENOME_WP3_{—}103 as PCR template. For this amplification specific primers were designed each containing a gene specific part as well as a nucleotide overhang for the addition of an restriction endonuclease recognition site (forward primer according to SEQ ID NO. 26 including NotI recognition site and the start codon; reverse primer according to SEQ ID NO. 27 including XmaI recognition site and the stop codon).

The PCR reaction was run using 100 ng of BAC DNA-template, 0.2 mM of each dNTP, 50 pmol forward primer, 50 pmol reverse primer, 1 U Phusion DNA polymerase (NEB) and 1× Phusion HF reaction buffer, following a cycle protocol as follows: 1 cycle of 60 seconds at 98° C., followed by 35 cycles of in each case 10 seconds at 98° C., 30 seconds at 55° C. and 60 at 72° C., followed by 1 cycle of 10 minutes at 72° C., then 4° C.

The resulting fragment was purified on an agarose gel, and subjected to a restriction digest using the restriction endonucleases XmaI and NotI.

As target vector the pIPKTA9 plasmid (Dong et al. (2006) Plant Cell 18(11): 3321-3331) was used. This vector is based on the pUC18-Vector and contains a CaMV 35S promotor and a 35S terminator which are separated by a multiple cloning site.

To insert the DNA sequence coding for RLK_compl_{—}3 Hcluster_{—}2 into the pIPKTA9 plasmid, the vector was cut within the multiple cloning site using the restriction endonuclease NotI and XmaI, followed by a purification on a agarose gel. Vector and PCR fragment were combined and subjected to ligation. After isolation of the resulting vector, correctness of sequence was confirmed by standard sequencing techniques.

### 2.2. Cloning of an Expression Vector Encoding RLK7

To obtain an expression vector for transient transformation of plant tissue, the RLK_{—}7 DNA sequence was amplified using the BAC clone BAC PHENOME_WP3_{—}104 as PCR template. For this amplification specific primers were designed each containing a gene specific part as well as a nucleotide overhang for the addition of an restriction endonuclease recognition site (forward primer according to SEQ ID No. 28, including a NotI recognition site and the start codon; reverse primer according to SEQ ID No. 29 including a XmaI recognition site and the stop codon).

The PCR reaction was run using 100 ng of BAC DNA-template, 0.2 mM of each dNTP, 50 pmol forward primer, 50 pmol reverse primer, 1 U Phusion DNA polymerase (NEB) and 1× Phusion HF reaction buffer, following a cycle protocol as follows: 1 cycle of 60 seconds at 98° C., followed by 35 cycles of in each case 10 seconds at 98° C., 30 seconds at 55° C. and 60 at 72° C., followed by 1 cycle of 10 minutes at 72° C., then 4° C.

The resulting fragment was purified on an agarose gel, and subjected to a restriction digest using the restriction endonucleases XmaI and NotI. As target vector the pIPKTA9 plasmid (Dong et al. (2006) Plant Cell 18(11): 3321-3331) was used. This vector is based on the pUC18-Vector and contains a CaMV 35S promotor and a 35S terminator which are separated by a multiple cloning site.

To insert the DNA sequence encoding RLK_{—}7 into the pIPKTA9 plasmid, the vector was cut within the multiple cloning site using the restriction endonucleases NotI and XmaI, followed by a purification on a agarose gel. Vector and PCR fragment were combined and subjected to ligation. After isolation of the resulting vector, correctness of sequence was confirmed by standard sequencing techniques.

All cloning steps such as restriction enzyme cleavages, agarose gel electrophoresis, purification of DNA fragments, ligation of DNA fragments, transformation of E. coli cells, bacterial cultures, and sequence analysis of recombinant DNA, were carried out as described in Sambrook et al. Cold Spring Harbor Laboratory Press (1989), 5 ISBN 0-87969-309-6.

### 2.3. Transformation and Results

Transient transformation and evaluation of resistance were performed as described in Example 1

Using this approach, the following results were obtained (Table 8):

| | | p two- | p one- | |
|---|---|---|---|---|
| transformed DNA | Rel. SI (%)^{a} | sided^{b} | sided | N^{c} |
| pIPKTA9^{d} | 100 | — | — | 5 |
| pJP01 (positive control)^{e} | 26.8 | 0.01272 | 0.00636 | 5 |
| VC-LTM59-38 comprising | 67.2 | 0.00357 | 0.00178 | 5 |
| SEQ ID NO. 1 (RLK7) | | | | |
| VC-LTM56-4 comprising | 67.2 | 0.06274 | 0.03137 | 5 |
| SEQ ID NO. 7 (cluster 2) | | | | |

^{a}relative susceptibility index in % of negative control
^{b}one-sample t-test relative to the hypothetical value 100
^{c}number of independent bombardements
^{d}empty expression vector containing the cauliflower mosaic virus 35S promoter and terminator flanking a multiple cloning site
^{e}vector expressing class III peroxidase cDNA sequence TaPrx103 from wheat under the control of the cauliflower mosaic virus 35S promoter and terminator

These results show that the expression of nucleic acid sequences of the present invention leads to an increased resistance of wheat to Blumeria graminis f.sp. tritici.

### 3. Stable Wheat Transformation

### 3.1. Generation of Constructs for Stable Wheat Transformation

The genomic DNAs encoding the receptor-like kinases RLK_compl_{—}3 Hcluster_{—}2 and RLK7 were generated by DNA synthesis (Geneart, Regensburg, Germany) in a way that an attB5-recombination site (Gateway system, (Invitrogen, Life Technologies, Carlsbad, Calif., USA)) is located upstream of the start-ATG and an attB4 recombination site is located downstream of the stop-codon. The synthesized DNAs were transferred to a pENTRY-B vector by using the BP reaction (Gateway system (Invitrogen, Life Technologies, Carlsbad, Calif., USA)) according to the protocol provided by the supplier.

To obtain the binary plant transformation vector, a triple LR reaction (Gateway system (Invitrogen, Life Technologies, Carlsbad, Calif., USA)) was performed according to the manufacturer's protocol by using a pENTRY-A vector containing a maize ubiquitine promoter (p-ZmUbi), the pENTRY-B vector containing the DNA coding for the receptor-like protein kinase and a pENTRY-C vector containing a Agrobacterium octopine synthase promoter (t-ocs) were used. As target a binary pDEST vector was used which is composed of: (1) a Kanamycin resistance cassette for bacterial selection (2) a pVS1 origin for replication in Agrobacteria (3) a pBR322 origin of replication for stable maintenance in E. coli and (4) between the right and left border an D-amino acid oxidase (GenBank U60066) under control of a pcUbi-promoter as D-aminoacid tolerance marker. The recombination reaction was transformed into E. coli (DH5alpha), mini-prepped and screened by specific restriction digestions. A positive clone from each vector construct was sequenced and submitted to wheat transformation.

### 3.2. Plant Material and Surface Sterilisation

A comprehensive discussion about wheat transformation methods and a protocol for the Agrobacterium-mediated transformation of wheat can be found in Jones et al. (2005) Plant Methods 1: 5.

Immature embryos (IEs) from Triticum aestivum (variety ‘Bobwhite’) are used as explant for Agrobacterium-mediated transformation. Donor plants are grown at 18-20° C. day and 14-16° C. night temperatures under a 16 h photoperiod (500-1000 μmolm-2s-1 photosynthetically active radiation (PAR)) with relative air humidity of 50-70% for approximately 8 to 11 weeks.

The optimal harvesting time is 12-20 days post-anthesis. For transformation IEs should be 0.8-1.5 mm in length and translucent in appearance. Donor plants used for harvesting should be at peak vigour to ensure optimal transformation and regeneration frequencies.

Immature seeds are surface sterilized by rinsing them 30-60 sec. in 70% (v/v) aqueous ethanol followed by 15 minutes 10% (v/v) Domestos bleach solution (Lever) gentle shaking. Then the immature seeds are rinsed 3-4 times with sterile distilled water and transferred to a sterile Petri dish, avoiding extreme dehydration. Immature seeds are ready for use.

### 3.3. Agrobacterium Culture

Agrobacterium cultures containing the vector harbouring a selectable marker (SM) cassette and the gene(s) of interests (GOI) described above are grown for 24-72 hours in a 28° C. incubator on LB agar plates with appropriate selection.

To obtain a liquid Agrobacterium culture one colony is picked from a 1-3 days old plate and re-suspended in liquid medium (5 g mannitol, 1 g L-glutamic acid, 250 mg KH₂PO₄, 100 mg NaCl, 100 mg MgSO₄×7H₂O, 5 g tryptone, 2.5 g yeast extract, pH 7.0, add after autoclave 1 μg biotin incl. appropriate antibiotics). Liquid culture is grown at 28° C. for ˜16 h to reach an OD₆₀₀ of ˜1. The Agrobacterium culture is centrifuged at 4.500 g for 10 minutes and resuspended in 4 ml inoculation medium (1/10 MS complete (30 g maltose, 100 mg MES; adjusted to pH 5.8 and add after autoclave 0.01% Pluronic, 200 μM acetosyringone to an OD₆₀₀ of ˜1. The Agrobacterium inoculation medium is ready to use.

### 3.4. Isolation of Immature Embryos (IEs)

The IEs are isolated from the immature seed followed by removing and discarding the embryo axis. The IEs are directly transferred in the Agrobacterium inoculation culture.

### 3.5. Co-Culture

Following isolation of immature embryos (IEs), the tube is vortexed at full speed for 10 seconds and IEs are allowed to settle in the solution for 30-60 minutes.

The Agrobacterium solution is removed and the IEs are placed on sterile Whatman filter paper #1 (4-5 pieces) to blot excess Agrobacterium solution. The top filter paper containing the IEs are transferred onto a plate containing approx. 20 ml of solidified co-culture media (1/10 MScomplete (30 g maltose, 0.69 g proline, 100 mg MES, 10 g Agar, adjust to pH 5.8, add after autoclave, 4 mg 2,4-D, 200 μM acetosyringone, 100 mg ascorbic acid)). The plates are sealed with parafilm and incubated for 2-3 days at 24° C. in the dark.

### 3.6. Callus Induction

Following co-culture, the explants are placed with the embryo axis facing down on recovery media (MS full complete (30 g maltose, 0.69 g proline, 20 mg thiamine, 1 g casein hydrolysate, 100 mg myo-inositol, 5 μM CuSO₄, 2.4 g NH₄NO₃, 1.95 g MES, 8 g Agar (Plant TC), adjust to pH 5.8 and add after autoclave 2 mg 2,4-D, 200 mg timentin, 100 mg ascorbic acid)) for 4 weeks at 24° C. in the dark. The calli are transferred to fresh recovery medium after two weeks.

### 3.7. Shoot Regeneration, Rooting and Selection

Calli are transferred to shoot regeneration medium (MS full complete (30 g maltose, 20 mg thiamine, 100 mg myo-inositol, 750 mg glutamine, 5 μM CuSO₄, 1.95 g MES; 8 g agar (Plant TC), adjust to pH 5.8 and add after autoclave, 0.5 mg TDZ, 200 mg timentin, 11 mM D-alanine) and are cultivated under light conditions at 21-25° C. for 3-4 weeks.

After shoot induction the explants are transferred to rooting media (1/2 MS complete, sucrose 30 g, agar 7 g and adjust to pH 5.8, add after autoclave, NAA 0.5 mg, timentin 200 mg, D-alanine 11 mM) in 100λ20 plates and are cultivated for 4-5 weeks at 21-25° C. under light conditions.

Putative transgenic shoots that develop roots are planted out into a nursery soil mix consisting of peat and sand (1:1) and maintained at 22-24° C. with elevated humidity (>70%) After two weeks, plants are removed from the humidity chamber and are further cultivated under greenhouse conditions.

### 4. Wheat Septoria Screening Assay

Transgenic plants are grown in the greenhouse at 19° C. and 60-80% humidity. After 11 days plants are inoculated with Septoria tritici spores (1.3×10⁶ Spores/ml in 0.1% Tween20 solution). Plants are incubated for 4 days at 19° C. and 80-90% humidity under long day conditions (16 h light). Plants are then grown for approx. 3 weeks at 19° C. and 60-80% humidity under long day conditions.

The diseased leaf area is scored by eye by trained personal. The percentage of the leaf area showing fungal pycnidia or strong yellowing/browning is considered as diseased leaf area. Per experiment the diseased leaf area of 16 transgenic plants (and 16 WT plants as control) is scored. For analysis the average of the diseased leaf area of the non-transgenic mother plant is set to 100% to calculate the relative diseased leaf area of the transgenic lines.

The expression of the receptor-like protein kinase will lead to enhanced resistance of wheat against Septoria tritici.

### 5. Wheat Rust Screening Assay

Transgenic plants are grown in the phytochamber at 22° C. and 75% humidity (16/8 h light/dark rhythm) for 2 weeks. The 2 weeks old plants are inoculated with wheat brown rust (Puccinia triticina) spores. Generally plants are inoculated with a 0.2% (w/v) spore suspension in HFE (Hydrofluoroether). Plants are incubated for 24 h in darkness under 100% humidity and 24° C. After the dark phase, plants are grown at 23° C., 75% humidity and a 16/8 hours light/dark rhythm

Diseased leaf area is scored by eye by trained personal. The percentage of the leaf area showing fungal colonies or strong yellowing/browning is considered as diseased leaf area. Per experiment the diseased leaf area of 16 transgenic plants (and 16 WT plants as control) is scored. For the analysis the average of the diseased leaf area of the non-transgenic mother plant is set to 100% to calculate the relative diseased leaf area of the transgenic lines.

The expression of the receptor-like protein kinase will lead to enhanced resistance of wheat against rust fungi.

### 6. Powdery Mildew Screening Assay

Transgenic plants are grown in the phytochamber at 22° C. and 75% humidity (16/8 h light/dark rhythm) for 2 weeks. The 2 weeks old plants are inoculated with spores of the powdery mildew fungus (Blumeria graminis f.sp. tritci). Generally inoculations with powdery mildew are performed with dry spores using an inoculation tower to a density of approx. 10 spores/mm². Plants are incubated for 7 days at 20° C., 75% humidity and a 16/8 hours light/dark rhythm.

Diseased leaf area is scored by eye by trained personal. The percentage of the leaf area showing white fungal colonies is considered as diseased leaf area. Per experiment the diseased leaf area of 16 transgenic plants (and 16 WT plants as control) is scored. For analysis the average of the diseased leaf area of the non-transgenic mother plant is set to 100% to calculate the relative diseased leaf area of the transgenic lines.

Expression of the receptor-like protein kinase will lead to enhanced resistance of wheat to powdery mildew fungus, in particular to Blumeria graminis f.sp. tritici.

1. A method of producing a transgenic plant cell, a transgenic plant or a transgenic part thereof having an increased resistance to pathogens compared to a control plant cell, plant or plant part, comprising increasing in the transgenic plant cell, the transgenic plant or the transgenic part thereof the content and/or activity of a receptor-like protein kinase which is encoded by a nucleic acid selected from the group consisting of:
(a) a nucleic acid comprising a nucleic acid sequence according to any of SEQ ID Nos. 1-5, 7-9, 11-13, 15-17, 19-21, 23 and 24 or a fragment of any of these sequences;
(b) a nucleic acid encoding a protein comprising the amino acid sequence according to any of SEQ ID Nos. 6, 10, 14, 18, 22 and 25;
(c) a nucleic acid comprising a nucleic acid sequence which is at least 70% identical to the sequence according to any of SEQ ID Nos. 1-5, 7-9, 11-13, 15-17, 19-21, 23 and 24 or a fragment of any of these sequences; and
(d) a nucleic acid hybridizing under stringent conditions with a nucleic acid comprising a nucleic acid sequence according to any of SEQ ID Nos. 1-5, 7-9, 11-13, 15-17, 19-21, 23 and 24 or a fragment of any of these sequences,
in comparison to the control plant cell, plant or plant part.

2. The method of claim 1, wherein the increasing comprises the steps of:
(a) introducing into a plant cell a vector which comprises:
(i) a promoter functional in plant cells;
(ii) operatively linked thereto the nucleic acid encoding the receptor-like protein kinase;
(iii) optionally, a termination sequence; and
(b) optionally, regenerating a transgenic plant from the transformed cell.

3. The method of claim 2, wherein the promoter is a tissue-specific and/or a pathogen-inducible promoter.

4. The method of claim 1, further comprising reducing the content and/or activity of at least one plant protein mediating pathogen susceptibility or increasing the content and/or activity of at least one other plant protein mediating pathogen resistance.

5. The method of claim 1, further comprising the step of crossing the transgenic plant with another plant in which the content and/or the activity of the receptor-like protein kinase is not increased and selecting transgenic progeny in which the content and/or the activity of the receptor-like protein kinase is increased.

6. A method of producing or obtaining mutant plants, plant cells or plant parts having an increased resistance to pathogens compared to control plants, plant cells or plant parts, comprising the steps of:
(a) mutagenizing plant material; and
(b) identifying plant material having at least one point mutation in an endogenous nucleic acid comprising a nucleic acid sequence having a sequence identity of at least 70% to the nucleic acid sequence according to any of SEQ ID Nos. 1-5, 7-9, 11-13, 15-17, 19-21, 23 and 24.

7. The method of claim 1, wherein the transgenic or mutant plant is a monocotyledonous plant.

8. The method of claim 7, wherein the monocotyledonous transgenic or mutant plant is a barley or [a] wheat plant.

9. The method of claim 1, wherein the transgenic or mutant plant has an increased resistance to a fungal pathogen.

10. The method of claim 9, wherein the transgenic or mutant plant has an increased resistance to Blumeria graminis, Septoria tritici and/or Puccinia triticina.

11. The method of claim 1, wherein the transgenic or mutant plant is a wheat plant and the pathogen is Blumeria graminis f.sp. tritici.

12. An expression construct comprising at least one nucleic acid selected from the group consisting of:
(a) a nucleic acid comprising a nucleic acid sequence according to any of SEQ ID Nos. 1-5, 7-9, 11-13, 15-17, 19-21, 23 and 24 or a fragment of any of these sequences;
(b) a nucleic acid encoding a protein comprising the amino acid sequence according to any of SEQ ID Nos. 6, 10, 14, 18, 22 and 25;
(c) a nucleic acid comprising a nucleic acid sequence which is at least 70% identical to the sequence according to any of SEQ ID Nos. 1-5, 7-9, 11-13, 15-17, 19-21, 23 and 24 or a fragment of any of these sequences; and
(d) a nucleic acid hybridizing under stringent conditions with a nucleic acid comprising a nucleic acid sequence according to any of SEQ ID Nos. 1-5, 7-9, 11-13, 15-17, 19-21, 23 and 24 or a fragment of any of these sequences.
operatively linked to a promoter functional in plant cells.

13. The expression construct of claim 12, further comprising regulatory sequences which can act as termination and/or polyadenylation signal in the plant cell and which are operably linked to the DNA sequence.

14. The expression construct of claim 12, wherein the promoter is a tissue-specific and/or [a] pathogen-inducible promoter.

15. A vector comprising the expression construct of claim 12.

16. A transgenic or mutant plant, plant cell or plant part with an increased resistance to pathogens compared to a control plant, plant cell or plant part, produced according to the method of claim 1.

17. (canceled)

18. A transgenic or mutant seed produced from the transgenic or mutant plant of claim 16.

19. Flour produced from the transgenic or mutant seed of claim 18, wherein the presence of the transgene or the mutation which increases the content and/or the activity of the receptor-like protein kinase can be detected in said flour.

20. (canceled)

21. A transgenic or mutant plant, plant cell or plant part with an increased resistance to pathogens compared to a control plant, plant cell or plant part, comprising the expression construct of claim 12.

22. A transgenic or mutant seed produced from the transgenic or mutant plant of claim 21.

23. A method for producing a true breeding plant comprising:
crossing the transgenic plant of claim 21 with another plant in which the content and/or the activity of the receptor-like protein kinase is not increased;
selecting transgenic progeny in which the content and/or the activity of the receptor-like protein kinase is increased; and
inbreeding the transgenic progeny and repeating the inbreeding step until a true breeding plant is obtained.


SEQUENCE LISTING (29 sequences)

SEQ ID NO: 1 - DNA, 3405 bp, Hordeum vulgare
atgggggacgggaagaggtactgttcctgctcctctacccatggcttctcggtgccgctaccgctcttctccttcttcaccttgttctgcctccatctgtccatcgccgccgccgccggcaacccgccccttcctctaaacaacacgcaggaatccatcatgagggatctctcgcgctctgtgggctcagccgggtggaacacaaccgtctccaatccatgcctatggaggggaatcggttgctccccttctaactccagttccttcttggtggtgaccaaagtcgatttatctggctactccatatccaactccacgctctttgcctccttatgctctctcgacaccttacactccctcgatctctccaataactatttcgccaatttgacgggccatttctctccttgccccatgactgccgggttgcgggtgctaaatttcagcagcaaccgtctgtcgggacgactaggccatctctccggtttctctcgactcgaggttcttgatctctccttcaattccttccttggtactgtgaccacccaattgagtgttatgcctaggttgaggagcctgaacctcagctccaattatttggtgggtgctgtccctctaagaatggctagctctatggaggagttggtgttgtctcgtaatagtttcagtgattcaattccaagcagtctgttcagttattcagacctcaccctgttggatcttagtcagaacagtttcactggtgatgtcgttgatgagttccggaagctgcccaagctcaggaccttaatcctttctggtaataacctgactggagcaataccggcgagcttgtcgaatgtcatgacgctcactcggtttgcggctaatcagaacaattttactggttctgtccctagtggtattacaaagcatgtgaagatgttggatttgagttataatggtctgagtggggagatcccctctgatctcctcggccccgcgggattggagactgttgatctctccagcaattacctccaaggaccaattcctaggaacttatcttcacacctctaccgcttgaggcttggtgataacaagctcaatgggaccatcccggacaccataggtgatgccttggccctggcttatcttgagttggacaacaatcacttgatgggaaatatctcttcacagcttggaagatgcaggaacttgtccttgttgaatctggcatcgaaccattttcagggtcaagtgcctgatgcaatcagtaaccttgacaagctggtagttctgaagcttcaaatgaacaatctcaggggatctatcccaagtacattttctgatttaacaaacctgaacactttgaatcttagtctgaattcattcacgggagagataccaatgggaattttcaaccttccaaagctttccaacttgaattttcaagggaacaagatcagcggtgccattccagtgtcagtcagttcattgcagtctctaattgagctcaatctggcagataattccctcactggtaccatcccaacaatgccgaccagtttgagcgcagttctcaatcttagtcacaatcatctcagtggatctattccttcaaatgtcggtgttttaaaagatttagagatccttgacctttcatacaacaacttgtctggtccagtgccatcctcacttgagggtctacagagcttgacaaagttggtgctatcttataatcacctctctgggtcccttcctgcattccgttcaagtgtgactgttgatagcactggaaatcctgatcttacaaatggcaaggaaagcagtaatgactctcctactagtaggaagacaaggacacatactgttgtcattattgttgctattgtcagttctcttgttggactgtgcttgctggctgctattgttatgttcgcattgtccaagagaatttatcgtgtggaagacgaaggaccatcaactgaagtgggtgtgtgtcaagtcattgacggccacttcataacaatgagcagtgtgcacacatctggaattgattttaggtatgcagtgaaagcagtctccaatcccaacaacatattcctgaagacaaggttctgcacctactacaaggccatgatgccaaatggatcaatctactctgtgaagaagcttgattggagtgacaagatattccacgttggtagccaagagaaatttggccatgagcttgaggtacttggcaagctaagcaattccagtgtcatggtgccattggcctatgtcttgacagaagacaatgcgtacctcctctatgagcatgtgtacaagagcacggtgttcgacttactacatgatggaaggtcacatgttctggactggacatcacggtatagcattgctttgggggtggcccaagggttgacctttcttcatgggcgcactcagccggttctgcttcttgatctgtcgacgaggactatccacttgaagtcaacaaacgagcctcagataggagatattgagctttacaaaattatcgatccttctaaaagtagcgggagcctttcaaccattgctggcacagtcggctatattccaccaggtaaaacacgtatcctcatattctgattcagtttgagcagctattgtcaacttgttgatttttctttgagaaatggatattttctgctatgttttatttacaaacacttcatgtcgtctgtttcttgtttgtagaaatttttcttaatgtgcttgactacaaatatgcgtgaatagtacctccaaattccacctgtcccattgtttggcctaaacttgaagtaccatcttatttcctgaagtgtttcagtggaatgctacctctttcactcgtttaatttagacacccaagcccatgctttatcagcgttatgttgaaagctgccatccaaactaaagtgtctcaactgggcaaataatagttcaaacggataactgctctgaggcaaacaagcttctcaacctgtgcccaagcccccgcgacaatcttgcagattttggttcagttatttcagtaactcagagaagttcatatagactaagatcaaatgtcaccgaattttgattgcagagtatgcatacaccatgaggttgacgatggctggcaacgtgtatagctttggagtcattttactggagctcttgacagggaaaccatctgtcagtgatggcatggagctagccaagtgggctctcagtctttcagctaggcctgagcaaagggagcaggtccttgacaccagggtctcaaggacttcagttggtgttcacagtcagatgctgtcagtcctcaatattgcgctctcctgtgttactttctctcccgatgctcggccgaagatgcgcaacgtcttaaggttgctcgccaacgcaaagtga

SEQ ID NO: 2 - DNA, 3365 bp, Hordeum vulgare
atgggggacgggaagaggtactgttcctgctcctctacccatggcttctcggtgccgctaccgctcttctccttcttcaccttgttctgcctccatctgtccatcgccgccgccgccggcaacccgccccttcctctaaacaacacgcaggaatccatcatgagggatctctcgcgctctgtgggctcagccgggtggaacacaaccgtctccaatccatgcctatggaggggaatcggttgctccccttctaactccagttccttcttggtggtgaccaaagtcgatttatctggctactccatatccaactccacgctctttgcctccttatgctctctcgacaccttacactccctcgatctctccaataactatttcgccaatttgacgggccatttctctccttgccccatgactgccgggttgcgggtgctaaatttcagcagcaaccgtctgtcgggacgactaggccatctctccggtttctctcgactcgaggttcttgatctctccttcaattccttccttggtactgtgaccacccaattgagtgttatgcctaggttgaggagcctgaacctcagctccaattatttggtgggtgctgtccctctaagaatggctagctctatggaggagttggtgttgtctcgtaatagtttcagtgattcaattccaagcagtctgttcagttattcagacctcaccctgttggatcttagtcagaacagtttcactggtgatgtcgttgatgagttccggaagctgcccaagctcaggaccttaatcctttctggtaataacctgactggagcaataccggcgagcttgtcgaatgtcatgacgctcactcggtttgcggctaatcagaacaattttactggttctgtccctagtggtattacaaagcatgtgaagatgttggatttgagttataatggtctgagtggggagatcccctctgatctcctcggccccgcgggattggagactgttgatctctccagcaattacctccaaggaccaattcctaggaacttatcttcacacctctaccgcttgaggcttggtgataacaagctcaatgggaccatcccggacaccataggtgatgccttggccctggcttatcttgagttggacaacaatcacttgatgggaaatatctcttcacagcttggaagatgcaggaacttgtccttgttgaatctggcatcgaaccattttcagggtcaagtgcctgatgcaatcagtaaccttgacaagctggtagttctgaagcttcaaatgaacaatctcaggggatctatcccaagtacattttctgatttaacaaacctgaacactttgaatcttagtctgaattcattcacgggagagataccaatgggaattttcaaccttccaaagctttccaacttgaattttcaagggaacaagatcagcggtgccattccagtgtcagtcagttcattgcagtctctaattgagctcaatctggcagataattccctcactggtaccatcccaacaatgccgaccagtttgagcgcagttctcaatcttagtcacaatcatctcagtggatctattccttcaaatgtcggtgttttaaaagatttagagatccttgacctttcatacaacaacttgtctggtccagtgccatcctcacttgagggtctacagagcttgacaaagttggtgctatcttataatcacctctctgggtcccttcctgcattccgttcaagtgtgactgttgatagcactggaaatcctgatcttacaaatggcaaggaaagcagtaatgactctcctactagtaggaagacaaggacacatactgttgtcattattgttgctattgtcagttctcttgttggactgtgcttgctggctgctattgttatgttcgcattgtccaagagaatttatcgtgtggaagacgaaggaccatcaactgaagtgggtgtgtgtcaagtcattgacggccacttcataacaatgagcagtgtgcacacatctggaattgattttaggtatgcagtgaaagcagtctccaatcccaacaacatattcctgaagacaaggttctgcacctactacaaggccatgatgccaaatggatcaatctactctgtgaagaagcttgattggagtgacaagatattccacgttggtagccaagagaaatttggccatgagcttgaggtacttggcaagctaagcaattccagtgtcatggtgccattggcctatgtcttgacagaagacaatgcgtacctcctctatgagcatgtgtacaagagcacggtgttcgacttactacatgatggaaggtcacatgttctggactggacatcacggtatagcattgctttgggggtggcccaagggttgacctttcttcatgggcgcactcagccggttctgcttcttgatctgtcgacgaggactatccacttgaagtcaacaaacgagcctcagataggagatattgagctttacaaaattatcgatccttctaaaagtagcgggagcctttcaaccattgctggcacagtcggctatattccaccaggtaaaacacgtatcctcatattctgattcagtttgagcagctattgtcaacttgttgatttttctttgagaaatggatattttctgctatgttttatttacaaacacttcatgtcgtctgtttcttgtttgtagaaatttttcttaatgtgcttgactacaaatatgcgtgaatagtacctccaaattccacctgtcccattgtttggcctaaacttgaagtaccatcttatttcctgaagtgtttcagtggaatgctacctctttcactcgtttaatttagacacccaagcccatgctttatcagcgttatgttgaaagctgccatccaaactaaagtgtctcaactgggcaaataatagttcaaacggataactgctctgaggcaaacaagcttctcaacctgtgcccaagcccccgcgacaatcttgcagattttggttcagttatttcagtaactcagagaagttcatatagactaagatcaaatgtcaccgaattttgattgcagagtatgcatacaccatgaggttgacgatggctggcaacgtgtatagctttggagtcattttactggagctcttgacagggaaaccatctgtcagtgatggcatggagctagccaagtgggctctcagtctttcagctaggcctgagcaaagggagcaggtccttgacaccagggtctcaaggacttcagttggtgttcacagtcagatgctgtcagtcctcaatattgcgctctcctgtgttactttctctcccgatgctcggccgaa

SEQ ID NO: 3 - DNA, 2895 bp, Hordeum vulgare
atgggggacgggaagaggtactgttcctgctcctctacccatggcttctcggtgccgctaccgctcttctccttcttcaccttgttctgcctccatctgtccatcgccgccgccgccggcaacccgccccttcctctaaacaacacgcaggaatccatcatgagggatctctcgcgctctgtgggctcagccgggtggaacacaaccgtctccaatccatgcctatggaggggaatcggttgctccccttctaactccagttccttcttggtggtgaccaaagtcgatttatctggctactccatatccaactccacgctctttgcctccttatgctctctcgacaccttacactccctcgatctctccaataactatttcgccaatttgacgggccatttctctccttgccccatgactgccgggttgcgggtgctaaatttcagcagcaaccgtctgtcgggacgactaggccatctctccggtttctctcgactcgaggttcttgatctctccttcaattccttccttggtactgtgaccacccaattgagtgttatgcctaggttgaggagcctgaacctcagctccaattatttggtgggtgctgtccctctaagaatggctagctctatggaggagttggtgttgtctcgtaatagtttcagtgattcaattccaagcagtctgttcagttattcagacctcaccctgttggatcttagtcagaacagtttcactggtgatgtcgttgatgagttccggaagctgcccaagctcaggaccttaatcctttctggtaataacctgactggagcaataccggcgagcttgtcgaatgtcatgacgctcactcggtttgcggctaatcagaacaattttactggttctgtccctagtggtattacaaagcatgtgaagatgttggatttgagttataatggtctgagtggggagatcccctctgatctcctcggccccgcgggattggagactgttgatctctccagcaattacctccaaggaccaattcctaggaacttatcttcacacctctaccgcttgaggcttggtgataacaagctcaatgggaccatcccggacaccataggtgatgccttggccctggcttatcttgagttggacaacaatcacttgatgggaaatatctcttcacagcttggaagatgcaggaacttgtccttgttgaatctggcatcgaaccattttcagggtcaagtgcctgatgcaatcagtaaccttgacaagctggtagttctgaagcttcaaatgaacaatctcaggggatctatcccaagtacattttctgatttaacaaacctgaacactttgaatcttagtctgaattcattcacgggagagataccaatgggaattttcaaccttccaaagctttccaacttgaattttcaagggaacaagatcagcggtgccattccagtgtcagtcagttcattgcagtctctaattgagctcaatctggcagataattccctcactggtaccatcccaacaatgccgaccagtttgagcgcagttctcaatcttagtcacaatcatctcagtggatctattccttcaaatgtcggtgttttaaaagatttagagatccttgacctttcatacaacaacttgtctggtccagtgccatcctcacttgagggtctacagagcttgacaaagttggtgctatcttataatcacctctctgggtcccttcctgcattccgttcaagtgtgactgttgatagcactggaaatcctgatcttacaaatggcaaggaaagcagtaatgactctcctactagtaggaagacaaggacacatactgttgtcattattgttgctattgtcagttctcttgttggactgtgcttgctggctgctattgttatgttcgcattgtccaagagaatttatcgtgtggaagacgaaggaccatcaactgaagtgggtgtgtgtcaagtcattgacggccacttcataacaatgagcagtgtgcacacatctggaattgattttaggtatgcagtgaaagcagtctccaatcccaacaacatattcctgaagacaaggttctgcacctactacaaggccatgatgccaaatggatcaatctactctgtgaagaagcttgattggagtgacaagatattccacgttggtagccaagagaaatttggccatgagcttgaggtacttggcaagctaagcaattccagtgtcatggtgccattggcctatgtcttgacagaagacaatgcgtacctcctctatgagcatgtgtacaagagcacggtgttcgacttactacatgatggaaggtcacatgttctggactggacatcacggtatagcattgctttgggggtggcccaagggttgacctttcttcatgggcgcactcagccggttctgcttcttgatctgtcgacgaggactatccacttgaagtcaacaaacgagcctcagataggagatattgagctttacaaaattatcgatccttctaaaagtagcgggagcctttcaaccattgctggcacagtcggctatattccaccagagtatgcatacaccatgaggttgacgatggctggcaacgtgtatagctttggagtcattttactggagctcttgacagggaaaccatctgtcagtgatggcatggagctagccaagtgggctctcagtctttcagctaggcctgagcaaagggagcaggtccttgacaccagggtctcaaggacttcagttggtgttcacagtcagatgctgtcagtcctcaatattgcgctctcctgtgttactttctctcccgatgctcggccgaagatgcgcaacgtcttaaggttgctcgccaacgcaaagtga

SEQ ID NO: 4 - DNA, 6515 bp, Hordeum vulgare
ttcggccgagcatcgggagagaaagtaacacaggagagcgcaatattgaggactgacagcatctgactgtgaacaccaactgaagtccttgagaccctggtgtcaaggacctgctccctttgctcaggcctagctgaaagactgagagcccacttggctagctccatgccatcactgacagatggtttccctgtcaagagctccagtaaaatgactccaaagctatacacgttgccagccatcgtcaacctcatggtgtatgcatactctgcaatcaaaattcggtgacatttgatcttagtctatatgaacttctctgagttactgaaataactgaaccaaaatctgcaagattgtcgcgggggcttgggcacaggttgagaagcttgtttgcctcagagcagttatccgtttgaactattatttgcccagttgagacactttagtttggatggcagctttcaacataacgctgataaagcatgggcttgggtgtctaaattaaacgagtgaaagaggtagcattccactgaaacacttcaggaaataagatggtacttcaagtttaggccaaacaatgggacaggtggaatttggaggtactattcacgcatatttgtagtcaagcacattaagaaaaatttctacaaacaagaaacagacgacatgaagtgtttgtaaataaaacatagcagaaaatatccatttctcaaagaaaaatcaacaagttgacaatagctgctcaaactgaatcagaatatgaggatacgtgttttacctggtggaatatagccgactgtgccagcaatggttgaaaggctcccgctacttttagaaggatcgataattttgtaaagctcaatatctcctatctgaggctcgtttgttgacttcaagtggatagtcctcgtcgacagatcaagaagcagaaccggctgagtgcgcccatgaagaaaggtcaacccttgggccacccccaaagcaatgctataccgtgatgtccagtccagaacatgtgaccttccatcatgtagtaagtcgaacaccgtgctcttgtacacatgctcatagaggaggtacgcattgtcttctgtcaagacataggccaatggcaccatgacactggaattgcttagcttgccaagtacctcaagctcatggccaaatttctcttggctaccaacgtggaatatcttgtcactccaatcaagcttcttcacagagtagattgatccatttggcatcatggccttgtagtaggtgcagaaccttgtcttcaggaatatgttgttgggattggagactgctttcactgcatacctaaaatcaattccagatgtgtgcacactgctcattgttatgaagtggccgtcaatgacttgacacacacccacttcagttgatggtccttcgtcttccacacgataaattctcttggacaatgcgaacataacaatagcagccagcaagcacagtccaacaagagaactgacaatagcaacaataatgacaacagtatgtgtccttgtcttcctactagtaggagagtcattactgctttccttgccatttgtaagatcaggatttccagtgctatcaacagtcacacttgaacggaatgcaggaagggacccagagaggtgattataagatagcaccaactttgtcaagctctgtagaccctcaagtgaggatggcactggaccagacaagttgttgtatgaaaggtcaaggatctctaaatcttttaaaacaccgacatttgaaggaatagatccactgagatgattgtgactaagattgagaactgcgctcaaactggtcggcattgttgggatggtaccagtgagggaattatctgccagattgagctcaattagagactgcaatgaactgactgacactggaatggcaccgctgatcttgttcccttgaaaattcaagttggaaagctttggaaggttgaaaattcccattggtatctctcccgtgaatgaattcagactaagattcaaagtgttcaggtttgttaaatcagaaaatgtacttgggatagatcccctgagattgttcatttgaagcttcagaactaccagcttgtcaaggttactgattgcatcaggcacttgaccctgaaaatggttcgatgccagattcaacaaggacaagttcctgcatcttccaagctgtgaagagatatttcccatcaagtgattgttgtccaactcaagataagccagggccaaggcatcacctatggtgtccgggatggtcccattgagcttgttatcaccaagcctcaagcggtagaggtgtgaagataagttcctaggaattggtccttggaggtaattgctggagagatcaacagtctccaatcccgcggggccgaggagatcagaggggatctccccactcagaccattataactcaaatccaacatcttcacatgctttgtaataccactagggacagaaccagtaaaattgttctgattagccgcaaaccgagtgagcgtcatgacattcgacaagctcgccggtattgctccagtcaggttattaccagaaaggattaaggtcctgagcttgggcagcttccggaactcatcaacgacatcaccagtgaaactgttctgactaagatccaacagggtgaggtctgaataactgaacagactgcttggaattgaatcactgaaactattacgagacaacaccaactcctccatagagctagccattcttagagggacagcacccaccaaataattggagctgaggttcaggctcctcaacctaggcataacactcaattgggtggtcacagtaccaaggaaggaattgaaggagagatcaagaacctcgagtcgagagaaaccggagagatggcctagtcgtcccgacagacggttgctgctgaaatttagcacccgcaacccggcagtcatggggcaaggagagaaatggcccgtcaaattggcgaaatagttattggagagatcgagggagtgtaaggtgtcgagagagcataaggaggcaaagagcgtggagttggatatggagtagccagataaatcgactttggtcaccaccaagaaggaactggagttagaaggggagcaaccgattcccctccataggcatggattggagacggttgtgttccacccggctgagcccacagagcgcgagagatccctcatgatggattcctgcgtgttgtttagaggaaggggcgggttgccggcggcggcggcgatggacagatggaggcagaacaaggtgaagaaggagaagagcggtagcggcaccgagaagccatgggtagaggagcaggaacagtacctcttcccgtcccccatggccggcgaccctcggccttttctcttgattcaattggctccctgctgatcctggccggctggagcaataggccaaagggactcccctaggcttcgggtttctgggaggccatggctgtgtggtgctcgtgccctcgggttttggtcgacatttgtggattcgagttcgagacgagggttgtagttgtagagagggaaaggggagactgcagttgtccagtctttgcctcttgcgagacggaagaggatcttctctgtttgggtccagagttttttttttttttttggtgggaaaaaaaaatctgagttgactgactcactcactcactgacgtatttggtggggtgaccaaaacctgagttttatttttcgaaagagaaaacacgaaacctgaggtgagaacccttgttgtgtgtggttttctcgcttggatttgcagttttgcatgctaaattctgagggccaatcatgaacgccatgtcttgcatgaaatttacttcatctgtatcataatagcattctgttctaaaataagtgttttaactttgtactatctctagtacaaagttgtattaaacttaaaacacttattttaaaacgaaggaagtacttgttattggaaaaaaaaactagtctggttctcatcaacaataaatatataggaacagcggaagaaatttactccctctgtatcgtattgtcgttggaaagaattagtctgattctttccaacaacaaatatttaggagcggaggaagaaatttactttctcaatatcacaatacttgttgaaaaaactagtctaatttttttcaactacaaatcttacctactaataaagcaaatattgcttctgccgtacgtcattcaaattgcccctaaagttcactaaaattacccatcaatgcctcctataagtcataaaaacgtttcaaacagaaaaatctccggactgggccggcccatgtaggcacctcctatattacgctctgggcgttggaaaaagatgcagcacacctgtttgggccagcccattcgtgggtgcctgtttttttggtttagttcttttatttttattttcagttccattttgtttttttatactttaaataatttagaacttcaactaacttttctaaattttaagaaactgagaatttctaaataaaatattcaaaaaaacataatttttttgagaattcaaaaactactcaggagttttgaaaaatgtttgcatatacaaaaaaatgtttaaattttgagaaattgtccataaaatatataattcaatgatttcaaacaaaagtccgtgtaaaaatcttaaaaagagtttgtgcctctgtttttagtctcgttttttattttaatttttccgttccattttttaatttaaataatttataactttgaaaaacttttgcaatttatgaaactgagaattttgaaataaaagtttgaagaaaacataaaatgtttgtgaattcaaaaaatactcggggtttttgtaaaaatattcgcatattcagaaaaacgttcataattttgagaaaaatgttggtgaaaacaataaaagtccatgatttttaaaaaaaatgtgtattattttttgagtgcaattgaaaaaaatgtttgctaattcaaaaaatgttcatgcatttcaaaaaatgtcctaaaattttaaagacataatataatcagcatcattggagatttaattgtttttctcccgttgcaacgcacgagcccttttgctattgtatcctaaactatgcaaccccggcctaaatcgaaccgaaacattgtgtcaactgggatttgtctaatgggccggcccgttttaaattttttgtttaaaaaatgttccaccaggtgcacatcctactccctacaagaccggcccacttaattttttccaaatgcctgcgtttcgcacagtggccagtccatttgctgtgtcacgatcgcgaaattaaaaaaaaaaggaagggtgacaccactctaactcggacttgcgacttcatagaggaaaacgcgttataatagtcggtgtgaccgactatttttttgtgtcacaattgcagaacatttgtgcaccttcggtaggttttacagtaaattgtgcaccttcggcaacggtttttccacaataagtacaacttgtatttaaaaacagcagaaatactattgtacgtactattgttccacgaatcgtagctaaaagcaacatcagttcatacaggtgtgaaatgcaaacgagattcttggaacaaaagagctagtatgtggagtataaaccatggaccacagaaaaaggacgcataacacgttggaacttggaaaatggaagaccatcttgaccaatcaaacacgttttggtgtggctctacctcaaaacagatcttactgatggactaggagggagaggtggaaccccgtctaacctcttttttttttgtccacaaaagtaaacaaagagaggatcactatcaaaaatagaaagaagagaaaacacaaactccgccctatttagcagactccataaaatgattcaacaacaaatttaatattagtgaaagaaaagaaatgattatgtatgtgatgcgactatttcaaaattccagttgtctactactccctccgtaccataatatatgacgttttagcagtttatttgaattgctaaaacgttatatattatgatacagaggtagtatataccaacaacaccatgtgaatattttaaataaaaataaataaacaccgcctgaaaagagcatgcaaaagtaaataaattgttggcaagtgttgggatttcttaaaaaatgggcaatactctctccctctcccccttaaatcatcttctcgtcgtcccatttgcacctgacctgacctagccccacaccatacttggaatgagactttgaatagcctcattatttttccttttgtgttgaaattgttaaaggatatcgttcagatcctagtttgttaggctcacggttatcctgagctgttaagtttatctctatctctctaactccaaacctcctgtaatctatctattggttaaacatcaccaacgaatccttgtaacccaagtcttgatcaatatataacctgcgggctgtcatgtatgacagttgagacgct

SEQ ID NO: 5 - DNA, 5132 bp, Hordeum vulgare
tttgaaacgtttttatgacttataggaggcattgatgggtaattttagtgaactttaggggcaatttgaatgacgtacggcagaagcaatatttgctttattagtaggtaagatttgtagttgaaaaaaattagactagttttttcaacaagtattgtgatattgagaaagtaaatttcttcctccgctcctaaatatttgttgttggaaagaatcagactaattctttccaacgacaatacgatacagagggagtaaatttcttccgctgttcctatatatttattgttgatgagaaccagactagtttttttttccaataacaagtacttccttcgttttaaaataagtgttttaagtttaatacaactttgtactagagatagtacaaagttaaaacacttattttagaacagaatgctattatgatacagatgaagtaaatttcatgcaagacatggcgttcatgattggccctcagaatttagcatgcaaaactgcaaatccaagcgagaaaaccacacacaacaagggttctcacctcaggtttcgtgttttctctttcgaaaaataaaactcaggttttggtcaccccaccaaatacgtcagtgagtgagtgagtcagtcaactcagatttttttttcccaccaaaaaaaaaaaaaaaaactctggacccaaacagagaagatcctcttccgtctcgcaagaggcaaagactggacaactgcagtctcccctttccctctctacaactacaaccctcgtctcgaactcgaatccacaaatgtcgaccaaaacccgagggcacgagcaccacacagccatggcctcccagaaacccgaagcctaggggagtccctttggcctattgctccagccggccaggatcagcagggagccaattgaatcaagagaaaaggccgagggtcgccggccatgggggacgggaagaggtactgttcctgctcctctacccatggcttctcggtgccgctaccgctcttctccttcttcaccttgttctgcctccatctgtccatcgccgccgccgccggcaacccgccccttcctctaaacaacacgcaggaatccatcatgagggatctctcgcgctctgtgggctcagccgggtggaacacaaccgtctccaatccatgcctatggaggggaatcggttgctccccttctaactccagttccttcttggtggtgaccaaagtcgatttatctggctactccatatccaactccacgctctttgcctccttatgctctctcgacaccttacactccctcgatctctccaataactatttcgccaatttgacgggccatttctctccttgccccatgactgccgggttgcgggtgctaaatttcagcagcaaccgtctgtcgggacgactaggccatctctccggtttctctcgactcgaggttcttgatctctccttcaattccttccttggtactgtgaccacccaattgagtgttatgcctaggttgaggagcctgaacctcagctccaattatttggtgggtgctgtccctctaagaatggctagctctatggaggagttggtgttgtctcgtaatagtttcagtgattcaattccaagcagtctgttcagttattcagacctcaccctgttggatcttagtcagaacagtttcactggtgatgtcgttgatgagttccggaagctgcccaagctcaggaccttaatcctttctggtaataacctgactggagcaataccggcgagcttgtcgaatgtcatgacgctcactcggtttgcggctaatcagaacaattttactggttctgtccctagtggtattacaaagcatgtgaagatgttggatttgagttataatggtctgagtggggagatcccctctgatctcctcggccccgcgggattggagactgttgatctctccagcaattacctccaaggaccaattcctaggaacttatcttcacacctctaccgcttgaggcttggtgataacaagctcaatgggaccatcccggacaccataggtgatgccttggccctggcttatcttgagttggacaacaatcacttgatgggaaatatctcttcacagcttggaagatgcaggaacttgtccttgttgaatctggcatcgaaccattttcagggtcaagtgcctgatgcaatcagtaaccttgacaagctggtagttctgaagcttcaaatgaacaatctcaggggatctatcccaagtacattttctgatttaacaaacctgaacactttgaatcttagtctgaattcattcacgggagagataccaatgggaattttcaaccttccaaagctttccaacttgaattttcaagggaacaagatcagcggtgccattccagtgtcagtcagttcattgcagtctctaattgagctcaatctggcagataattccctcactggtaccatcccaacaatgccgaccagtttgagcgcagttctcaatcttagtcacaatcatctcagtggatctattccttcaaatgtcggtgttttaaaagatttagagatccttgacctttcatacaacaacttgtctggtccagtgccatcctcacttgagggtctacagagcttgacaaagttggtgctatcttataatcacctctctgggtcccttcctgcattccgttcaagtgtgactgttgatagcactggaaatcctgatcttacaaatggcaaggaaagcagtaatgactctcctactagtaggaagacaaggacacatactgttgtcattattgttgctattgtcagttctcttgttggactgtgcttgctggctgctattgttatgttcgcattgtccaagagaatttatcgtgtggaagacgaaggaccatcaactgaagtgggtgtgtgtcaagtcattgacggccacttcataacaatgagcagtgtgcacacatctggaattgattttaggtatgcagtgaaagcagtctccaatcccaacaacatattcctgaagacaaggttctgcacctactacaaggccatgatgccaaatggatcaatctactctgtgaagaagcttgattggagtgacaagatattccacgttggtagccaagagaaatttggccatgagcttgaggtacttggcaagctaagcaattccagtgtcatggtgccattggcctatgtcttgacagaagacaatgcgtacctcctctatgagcatgtgtacaagagcacggtgttcgacttactacatgatggaaggtcacatgttctggactggacatcacggtatagcattgctttgggggtggcccaagggttgacctttcttcatgggcgcactcagccggttctgcttcttgatctgtcgacgaggactatccacttgaagtcaacaaacgagcctcagataggagatattgagctttacaaaattatcgatccttctaaaagtagcgggagcctttcaaccattgctggcacagtcggctatattccaccaggtaaaacacgtatcctcatattctgattcagtttgagcagctattgtcaacttgttgatttttctttgagaaatggatattttctgctatgttttatttacaaacacttcatgtcgtctgtttcttgtttgtagaaatttttcttaatgtgcttgactacaaatatgcgtgaatagtacctccaaattccacctgtcccattgtttggcctaaacttgaagtaccatcttatttcctgaagtgtttcagtggaatgctacctctttcactcgtttaatttagacacccaagcccatgctttatcagcgttatgttgaaagctgccatccaaactaaagtgtctcaactgggcaaataatagttcaaacggataactgctctgaggcaaacaagcttctcaacctgtgcccaagcccccgcgacaatcttgcagattttggttcagttatttcagtaactcagagaagttcatatagactaagatcaaatgtcaccgaattttgattgcagagtatgcatacaccatgaggttgacgatggctggcaacgtgtatagctttggagtcattttactggagctcttgacagggaaaccatctgtcagtgatggcatggagctagccaagtgggctctcagtctttcagctaggcctgagcaaagggagcaggtccttgacaccagggtctcaaggacttcagttggtgttcacagtcagatgctgtcagtcctcaatattgcgctctcctgtgttactttctctcccgatgctcggccgaagatgcgcaacgtcttaaggttgctcgccaacgcaaagtgaatgtgaatcaaaagaaagcagtcggttttggtaggttagataagttctggatggaatctgacattttcttagtttgtggatgtcagtatacaggtgatcattgtatagtgacatagtctatggtaggttatgagttggtgacgagtttgaattgttaagtgtatattttttggactgaacgatctgtgatctttaaacaccacagtgatggttattagcagacttgagtgcattgttttcttgttaattcgactagtactttgaattgataactagtaatcatgcacgtgcaacgcatgtgtcgtcatcaaatataaatgtgagtatgctcaaaggtatactcaaaggacctggaatttaaaatccatgtcctagtataatttataaaagatgttcataaagaggaatgtatatacaaatattctagaatgagcttttggagataaatttcaattgaacatacgaatcttaatattcctttcaaaaaccaataaatatattcagcaaaacccatgccaggtgtatgccaagtgcactttaccaaggtttacagccggcaaaccctatgccgggtacatatctaccttcgtggggcgctcatcatagaatgatttttttgtagtgcatttgatgaaagaaaaattacaacatcacaaactgcagggtccttataaaggtcgaagaggcatctaataataaggtgaaaataatgaagttacatctgcttttaacacaatatatatgtacgtccaccaaatacaagtaggactactcaaacaagacttcaaat

SEQ ID NO: 6 - protein, 964 aa, Hordeum vulgare
MGDGKRYCSCSSTHGFSVPLPLFSFFTLFCLHLSIAAAAGNPPLPLNNTQESIMRDLSRSVGSAGWNTTVSNPCLWRGIGCSPSNSSSFLVVTKVDLSGYSISNSTLFASLCSLDTLHSLDLSNNYFANLTGHFSPCPMTAGLRVLNFSSNRLSGRLGHLSGFSRLEVLDLSFNSFLGTVTTQLSVMPRLRSLNLSSNYLVGAVPLRMASSMEELVLSRNSFSDSIPSSLFSYSDLTLLDLSQNSFTGDVVDEFRKLPKLRTLILSGNNLTGAIPASLSNVMTLTRFAANQNNFTGSVPSGITKHVKMLDLSYNGLSGEIPSDLLGPAGLETVDLSSNYLQGPIPRNLSSHLYRLRLGDNKLNGTIPDTIGDALALAYLELDNNHLMGNISSQLGRCRNLSLLNLASNHFQGQVPDAISNLDKLVVLKLQMNNLRGSIPSTFSDLTNLNTLNLSLNSFTGEIPMGIFNLPKLSNLNFQGNKISGAIPVSVSSLQSLIELNLADNSLTGTIPTMPTSLSAVLNLSHNHLSGSIPSNVGVLKDLEILDLSYNNLSGPVPSSLEGLQSLTKLVLSYNHLSGSLPAFRSSVTVDSTGNPDLTNGKESSNDSPTSRKTRTHTVVIIVAIVSSLVGLCLLAAIVMFALSKRIYRVEDEGPSTEVGVCQVIDGHFITMSSVHTSGIDFRYAVKAVSNPNNIFLKTRFCTYYKAMMPNGSIYSVKKLDWSDKIFHVGSQEKFGHELEVLGKLSNSSVMVPLAYVLTEDNAYLLYEHVYKSTVFDLLHDGRSHVLDWTSRYSIALGVAQGLTFLHGRTQPVLLLDLSTRTIHLKSTNEPQIGDIELYKIIDPSKSSGSLSTIAGTVGYIPPEYAYTMRLTMAGNVYSFGVILLELLTGKPSVSDGMELAKWALSLSARPEQREQVLDTRVSRTSVGVHSQMLSVLNIALSCVTFSPDARPKMRNVLRLLANAK

SEQ ID NO: 7 - DNA, 2138 bp, Hordeum vulgare
atgcccactgcccttaaggccttaactctcttgtttgtgcttgcagtccttgcaccagatcagtcagaaggacggcatcatcggcgtgattgtccttctttctcgtgcggccctcatggaaatgtatcgtctccgtttcgtcgggcaagtgatccacctggctgcggctataaatcttatgagctggtttgcagtgatactaaggctataattcgcatccgaagtgcgccatactatgtgtctggcatcaactacaacgatgctaccttctgggtacttgatgccgacctggatttatacagcagctgtcctctgcctcaatggaatcctccttctcatcttacagatgtaaaccaaggcatggtcgaattgtcccccgttagttatactgaagcttgttttgtggacaatgagatgtacgtccttgttgcttgcctgagcaccaatgattcttttgtttacgtgttaactggctatggagtgtgtggaatcgagtaccttgagctttcttgtgggtacttgtccatgactcgtctaacttgggaggtcgacgttcagcagccgctatataatgtaagttatgcagatgtcgtgaaatccatgaggagtggattcgccattcgatttccttatcctataactcttaggaggatgagcttcgatgaatgcctatttctttggacattgtaagttcaggattttgacatttttacaccatggttatttgttggcacccgtcaaccaaaaactgttgtgcccctcactcatcctcttttccataccctcacatggtattgcagcggattccatgggatgacaattccaacttcgattgtacacattcttcaatatcctggagttctcttggggtgtgctggattatttgaaaacggaggactcgtagaacaggtcacgtggatcctgacgtttattgctggtacttcagtattttattttcttaatcaattgcagtttctaaataatcacagttctgtattcacgtacccacataatgactgtataaaccattcaatacagtgacatgcagatatgttttggcaccactgtctgtgttgatcttcttggcctacaagtactggcaagcaaggatcacgatcgatgcagtcgagagatttctccggatgcaacaagtgcttggcccgaggaggtatgcttacacagacatcaccgcgatcacaggccatttcagagacaagctgggccaaggtgggtatgggtccgtgtacaagggtgtgctgctcccaggcaatgtccatgtggccatcaagatgctggtgagcagctctaattgtaatggagaagattttatcagtcaggtttcaaccattggcaggatccaccatgttaatgtggtgcgtttagtgggcttctgcccggaggaactgagacaagcactcgtctatgagtacatgtcaggaggttctctagacaagtacatcttctcagttgagaggagtttttcctgggataagctcgccgagattgctttgggcatcgccaggggtatcgactacctgcatcaggggtgcgagatgcagattctacactttgatatcaagccgcacaacatccttcttgacagcaattttgtcccaaaaattgctgatttcggtctcgccaaactatacccaagggacaacagctttgtgccatcgagagctctacggggaacaattgggtacatagctcctgagatgatatccaggagctttggtgtcatatcgagcaagtctgacgtttacagctttggaatgttgttgttggagatggctggaggaagaaggaacgctgatccaaatgcggcgaactcaagccagtcttactacccatcatgggtgtatgacaaactaactgctccagtagtagatgcgatatgtccagttgcaagcatgcatgaattggagaagaagctgtgcatcgttggattatggtgcattcagatgaagtcacatgacaggccaacgatgagcgaggtcatagacatgttggaaggtggctttgatggcctgcagatgccttccaggccattcttctgcgacgacgatcacaccgctgtcccggattcttaccctttgttgtccgagctgacagagatctcggaggaggatgatgagtag

SEQ ID NO: 8 - DNA, 1797 bp, Hordeum vulgare
atgcccactgcccttaaggccttaactctcttgtttgtgcttgcagtccttgcaccagatcagtcagaaggacggcatcatcggcgtgattgtccttctttctcgtgcggccctcatggaaatgtatcgtctccgtttcgtcgggcaagtgatccacctggctgcggctataaatcttatgagctggtttgcagtgatactaaggctataattcgcatccgaagtgcgccatactatgtgtctggcatcaactacaacgatgctaccttctgggtacttgatgccgacctggatttatacagcagctgtcctctgcctcaatggaatcctccttctcatcttacagatgtaaaccaaggcatggtcgaattgtcccccgttagttatactgaagcttgttttgtggacaatgagatgtacgtccttgttgcttgcctgagcaccaatgattcttttgtttacgtgttaactggctatggagtgtgtggaatcgagtaccttgagctttcttgtgggtacttgtccatgactcgtctaacttgggaggtcgacgttcagcagccgctatataatgtaagttatgcagatgtcgtgaaatccatgaggagtggattcgccattcgatttccttatcctataactcttaggttcgattgtacacattcttcaatatcctggagttctcttggggtgtgctggcttcagtattttattttggcaccactgtctgtgttgatcttcttggcctacaagtactggcaagcaaggatcacgatcgatgcagtcgagagatttctccggatgcaacaagtgcttggcccgaggaggtatgcttacacagacatcaccgcgatcacaggccatttcagagacaagctgggccaaggtgggtatgggtccgtgtacaagggtgtgctgctcccaggcaatgtccatgtggccatcaagatgctggtgagcagctctaattgtaatggagaagattttatcagtcaggtttcaaccattggcaggatccaccatgttaatgtggtgcgtttagtgggcttctgcccggaggaactgagacaagcactcgtctatgagtacatgtcaggaggttctctagacaagtacatcttctcagttgagaggagtttttcctgggataagctcgccgagattgctttgggcatcgccaggggtatcgactacctgcatcaggggtgcgagatgcagattctacactttgatatcaagccgcacaacatccttcttgacagcaattttgtcccaaaaattgctgatttcggtctcgccaaactatacccaagggacaacagctttgtgccatcgagagctctacggggaacaattgggtacatagctcctgagatgatatccaggagctttggtgtcatatcgagcaagtctgacgtttacagctttggaatgttgttgttggagatggctggaggaagaaggaacgctgatccaaatgcggcgaactcaagccagtcttactacccatcatgggtgtatgacaaactaactgctccagtagtagatgcgatatgtccagttgcaagcatgcatgaattggagaagaagctgtgcatcgttggattatggtgcattcagatgaagtcacatgacaggccaacgatgagcgaggtcatagacatgttggaaggtggctttgatggcctgcagatgccttccaggccattcttctgcgacgacgatcacaccgctgtcccggattcttaccctttgttgtccgagctgacagagatctcggaggaggatgatgagtag

SEQ ID NO: 9 - DNA, 18976 bp, Hordeum vulgare
atcagaaaccgctctctcgagcgcacgcaccgttactaaagggaacttttgttcccactcaacactggtaagaacccggtccagcttttcgaaagtctggttttgtagggaattcgcccatgtgaatttcctaccagagagttctatctcccttagatccaggctctcgataatagtattaaacataaatggccatctgccatcaaaattatcattgctctttttgtcagctctcctaataatgttgaaatcacccccaatcaaaagtggcaatttctcatcgcagatccgaaccagatcggcgagaaaatcaggtttaagctctggctgagcagccccatacacagcaatgagagcccattggaatccatccactttagatgtaatcctgaatttaaccgcaaaatctcctaataccacttctctaacctgaagggtatcacatctaactccaagtaaaatcccaccggatcttcctcttggagggagacaatgcgagtcaaaatctactcccactgataaagaactcaaaaactgagaagtaaaattatctctcccactctccatgagtgctataaaatcaaggtgatgctctaaagtagcctcagcaagaaatcttattttagccaagtcccgaagacctctgctattccaaaacattcctctcatctttcatcatagaattttttagcagtcttaaatcgagcactccttctaaccgcagaaaccgggtatagtttccgtttccaaggccgttttggtttgtccccaaaaccctgatccatataaccaagtatatgtgagtcaaccaccaaagattgtacctccttggcactgtgctctaggacattgtcctctcccacctctgaatcggccagtatgtccaaattctcacataaagtattcagaccccccaactcattaatatcagaatcactcataggtttggcggcagcaaaattacgaataatattaagagccctgtctacctctaagtatagaaggtcattaatagatttcgaaatttgtctaccagtagtacccaaagacaccccaacaacaacaacattatgaataatatcattcggagaaaaatgaaggttggaattgtctttgttaaaagacatacctgtcgaagtttccgcatccttcatctttgcagctcgcattgcacgccccaactgcaagtcatccgcatccggttgttcctgaatacgctgactagaccgcctgtcccgagtcatcggattcgggagccccccaaacgctataacatcatccgcagagaaatgcgcaccgtcatccaccaccgtcccaccaatagagcactcagtccctgttgccataaccggcactgaagcctccactgcgacaaccttaggtgttttagtagcagccagattctgatcattcatgaaccccaagtcctccttccttgcttctgcaagatatgtgtcaagagagacaacaggcaatcccaccgtaggcgaaactggcccctgcgcatcctcgatgggcagcttccccgggaaaagggtcttacgccgctgcccctcgaagcatgccaccaggcggccacctccggccttggccggagccgatgccggaccaaaagtgccaaaccggagctgcgccagcggagccgaaatggaaacagcacccccttcgagcccacctgtgaaggcgccggttccttagcgttaatgtgcttgttgttgctcttgtcggcctcccttgcccccgagtcatcatccccatcaggcatatccacatccttttcttgcactccttcctcaaataaaggaggactctcaatctcaagctggagggtttatctcatacccgcatatgtccaaaccacttcatctggcacgagtgcaatgtcaatgacactaacaagcacacaagcaactcccttagctcaggtaaacaacatgtctaccttttcggtcttgccaatcaaagatcccaaactccaggtaaccagaaagtcgctcatagccttagaaggtgccccagaaaatcgaacccaaatctgaggaagagccacaccttgtggctcctcactcgtccacgcatcaaactccaagacgcaactagtgcttggcactctgcacatgccaaacttaagcaatcacgccaggtcctccttagtgggaaacacgactttgaacacgttctcagcaatcaacactggctcccatcgaaaagtcaaagaaaccagctcccggagctgctgcaccacctgctcagcagtcatattcccgcgagtcacggtgaccgtacccataagtgacgcatcaggcacgtggaccgtagtcgaaaccataggggactcaaagaacatcagctcctggcagcaaaccccatagatagtcacgacaggcattggtccaaccgtaagagggcacttagctgtgccatgcttgggcttaaggaaaaactcacaaagatccgccttgcactcagatacaaagtgaccagtctctccacatcgatagcatgtcattttctcctttttgcgggcccacttggaaggacgatcacctccctcgagccgcccttgtgagatgctcttgtctgcctctccaagcggtccaggaacaaccacctgaggctgcaccgtggagttggagccttccatggcctgccccgctgtaggctccgttggagtagctaccaccgccacagcagcaacagggacaactgaagggggaagcggaggcttaggcttcctgccacctcctcttccaccccaattttgtcggaaagcgcccctcttaggatgctgaggccccgccacccctggtgcaaacttcccaggaggaccggagaaacccgcactgccgccatgattctgccacgcatagccgcgtcccccacctgccgaggaagaaccacaatgttgtccctcaccgtagacgtcaaaaccgtcatcaccccatcgccctcggggtggctgcctgtccacgccgccggcccggtaggagcagccgaagccgccgctggcttcgccggcttagcaggaggacggagaccctggccagtgggacggcccgggtgctgcaagccctggccaccgcccctccctggaccgtccgctgcgtcggccttgaacgatggagcacctcgccccgccatctcggacgagggccgcagagggagcacccgagcagctcgaccagacccaagctttggaaaacccttggtaagcggaacacgaaccctagatggcggcggtacgtgagatacttcgggactgcatgcagattctaccgtagcaggcacgttctcatcctgggccatatacccaggaggctgagaacccgaaagcaaagaaggcccgtcatagtccacgtccgatggtgccgcatacaaaaaacgtatccttgaatcacgcgctaacgactgcactgcagccgccgccgacgacgccccgccaccgcggccaatcttcttgttcttcgagatcaccgtccatgtctccggaatgaaatcagacaaagagaggggtggaaggcttaccttcggaatcggaccaacccatggtttaacgccatttgaaaccctcgtcctcttatcaagtttaaaatcaggccgacgcttgcgctcaaaatacgccgcctccggcatcgtctggcatggataatgagcaggcacctccgtcggactcgcgctggactgatcgtcatcttcctcgtcttcgagatctgcaagagcccaaaagcgtccaccaaaccgattaggagatgttaggtcaacggaaacaaactccttcttacacatcaatggcagaatcggattctgcaccaccaccctaaaaccatcgatctccatagcctgtccgaccgatggacactgacgctcgagcagataccgaccagccagaatgtcatcatccacatcccccaccgtgatccaaaaagagatcatgtgaagggatatgaaacccggcagaggactgtgagaggcaatggtggtatcctcatacagcacctcccaacaacggtatggagctccggcaggatcctgatccatccctcctgagattggatcaacccagccaacttccgccacattctcagatgcagcagactcgctggacgcggatccagaagtgcatggcgttgcagaagctgcgcctgagatctcccgaaacgccccgccgtaggcctgcccgaacgcctccccgccggtggcctgcccgaacgcctcacccgcgacgccgggaatctccggcagcgccctgccggaggcccgatcgatcttgtcgggcggcgccccagagtcgccagactccgcccgtatcgccttaggcatcgccacgtacatgatcattacttggccggtacctatgtcatcatcattacttgggacgtggatagctactgatcagatatttgctcttcaatagccggtcacattcaggggaggaaaaaagccttccacatatttgtttatttttagtattctatcggtcgtgcgcagttgaaatgctttcctttctaaattataatttgcagacaatagaaaccaggggaaatatcaaatgaatctgcacatataatgtgcacttggtgctaaagatcttaagaatttttttggaagttagttaaaaaaaagcagaaaaaacatgccttgcgccgcactaggaagcaacaaaccatccgggatgtcttggccgactaccgctggatcgcgacatcagggggcggcggggggcttgaacccctgggcttttggcagtatttccaggtcttgcagttggttcgtagggttcagctatctacctttgactgacattctcgtatggcgttggaggtcagatgaccagtacacgtccaacagttgctacgacaccctctttcaagtaagagttggttcgagctcttggaaactaaagtggaattcttgggctccatcatttataagtagatatttataagcaaattttggtaagttagttctgtagaggaggaggtctggaatggagaacatcaccatcacctagatgagccagtaatcggtatgtatttactcaactttggctttgtcggcaagtagctaagtctcctagtcagcaatgtcacagtcatgtagagggcactagttggcatgtattgaccctctcttggagttttatttgtaactttgacagccagtcccttatttagccattggtctgcacgtctgtgctcctgtgtaccagtctgccatggcgatgcccactgcccttaaggccttaactctcttgtttgtgcttgcagtccttgcaccagatcagtcagaaggacggcatcatcggcgtgattgtccttctttctcgtgcggccctcatggaaatgtatcgtctccgtttcgtcgggcaagtgatccacctggctgcggctataaatcttatgagctggtttgcagtgatactaaggctataattcgcatccgaagtgcgccatactatgtgtctggcatcaactacaacgatgctaccttctgggtacttgatgccgacctggatttatacagcagctgtcctctgcctcaatggaatcctccttctcatcttacagatgtaaaccaaggcatggtcgaattgtcccccgttagttatactgaagcttgttttgtggacaatgagatgtacgtccttgttgcttgcctgagcaccaatgattcttttgtttacgtgttaactggctatggagtgtgtggaatcgagtaccttgagctttcttgtgggtacttgtccatgactcgtctaacttgggaggtcgacgttcagcagccgctatataatgtaagttatgcagatgtcgtgaaatccatgaggagtggattcgccattcgatttccttatcctataactcttaggaggatgagcttcgatgaatgcctatttctttggacattgtaagttcaggattttgacatttttacaccatggttatttgttggcacccgtcaaccaaaaactgttgtgcccctcactcatcctcttttccataccctcacatggtattgcagcggattccatgggatgacaattccaacttcgattgtacacattcttcaatatcctggagttctcttggggtgtgctggattatttgaaaacggaggactcgtagaacaggtcacgtggatcctgacgtttattgctggtacttcagtattttattttcttaatcaattgcagtttctaaataatcacagttctgtattcacgtacccacataatgactgtataaaccattcaatacagtgacatgcagatatgttttggcaccactgtctgtgttgatcttcttggcctacaagtactggcaagcaaggatcacgatcgatgcagtcgagagatttctccggatgcaacaagtgcttggcccgaggaggtatgcttacacagacatcaccgcgatcacaggccatttcagagacaagctgggccaaggtgggtatgggtccgtgtacaagggtgtgctgctcccaggcaatgtccatgtggccatcaagatgctggtgagcagctctaattgtaatggagaagattttatcagtcaggtttcaaccattggcaggatccaccatgttaatgtggtgcgtttagtgggcttctgcccggaggaactgagacaagcactcgtctatgagtacatgtcaggaggttctctagacaagtacatcttctcagttgagaggagtttttcctgggataagctcgccgagattgctttgggcatcgccaggggtatcgactacctgcatcaggggtgcgagatgcagattctacactttgatatcaagccgcacaacatccttcttgacagcaattttgtcccaaaaattgctgatttcggtctcgccaaactatacccaagggacaacagctttgtgccatcgagagctctacggggaacaattgggtacatagctcctgagatgatatccaggagctttggtgtcatatcgagcaagtctgacgtttacagctttggaatgttgttgttggagatggctggaggaagaaggaacgctgatccaaatgcggcgaactcaagccagtcttactacccatcatgggtgtatgacaaactaactgctccagtagtagatgcgatatgtccagttgcaagcatgcatgaattggagaagaagctgtgcatcgttggattatggtgcattcagatgaagtcacatgacaggccaacgatgagcgaggtcatagacatgttggaaggtggctttgatggcctgcagatgccttccaggccattcttctgcgacgacgatcacaccgctgtcccggattcttaccctttgttgtccgagctgacagagatctcggaggaggatgatgagtagcatgtgtgtgtgtgtgtgtgtgtgatttaatgtctcaagtcttacgaaataacctactattatgttttcccagacatgtgaagatttagatctccaattcctccttatcaaaaaaaaatgtagatctccaactgttaattgaaacgtaattatcttcctggaagacacacgaaaagcacgatgtaaaaaatgcagagtactctgttttcttcagctgctgatgatgctcgaaatttttggagtgggtatgaacagacccagcctgtcctgtatgaagtatgaacccttgtgattgataaagcttgggctcagtgtatggagatgatgatgattgcagtggaatttgtgtggacgacggtgttcacacttttatctatttttcctaaggccaaattcttgtaaccatgaatgtgattgtttacctgcagtccaatgtcgcatactcgcatatggaccaaactagttgacagcagcagcctcaccgtcagctcagggtgagaaaaacttaagctgattttttgctgtcacacgcattctgatttaatcgacggcaatttcaacggtataaaaatcattcaaatgactttgaagttcagcagaaaactactgtacaaaccaagaatacaacctcctgccgtatgcgttcctgcgattacgagtgcgattgtagactcgctgtcgtccagtcccacatcttcagccgttgcgactacgacgagtgctctggctcatatgaaccactgcaaatgactggacagcggcagcaacctcaaccccagggcacctcgtactctgtcatccaaatttctacagcaataatttcagacatgtaaacgtcgaaacgacttgtagaccgagaacaaattgccaacacattccgatttaatatgtgttaatttcagccgtataaaagacattcagatgacttggaagtccagcaaaaaaaaaaaaaaactaaaagctaagaatgtgacctcctgccgtatgtgtcccgcatcttcggtagagaatcttgaatgtgactgtagacttgctctgaaaattccaggccgagtgtcatcttcatcatgtaaatttctgcgaaaatgatgattccttccattacaaaatcagcggatatcagtagcagcgtcagtctcagggtaagttcaaaacaaaagtaaagcatccatcaagtccagtcatataagcgttccagccgagatgacttggaatttcagtcagacttctgataggtcttgaggaataattcctctcgagatcaatccaaagctcctctagtgttcatgcggcccatatggaccattgcaaaagcagtggacaacagtagctacctcagacctcaatcagtctcagtgcaaaaaaaaatccgttcacataaaccaatgttttaaatagcgggctacggcaaatagcggcaaacctctaaatcagctatagcggcatatttgtacatgataccatttagcggtaccctgctgaaaaggctataacgaggttatagcggggctatagcgggctatttaaaactatgcgtaaagcacacttgaaatgacttgcagttgcaagctcaacagagaattacagcagctaagaattcctgcaattttgactgtgattgtagactcgctgtcgtccagtcctgcttcttcagcagcaaaagctagggctaaatagtcatatggctcaacatgtggaaccataaaaatgtgcaatttgccgatcaagcactctctgcttttaaagaaaatatctagagcaacttcctggaatactgttagttcagaccaaataacattttccccctacattgcatcagttatttccatcatcgttgcacttctctcatcgattccctgctattagtctcatggcaaattaagaaaaagggagggggcgcgggtggatcatgatcacatcaggtggacagcgctggcatgctcttcaaacaaaaactgagaaacacactcaaacacacaagttttggtgatggccctgtttggatcctctaacttagctagaggttagagttagtttctagctcatgactaaccctaaactaactctagccaaagaggtgtttggatgaaagggttagattgacaatagatgcactttatggaaagagaaaaatgattttttaatggaccccatgagtactagctccaattagcatctcttggctggtaggagagtgagagaaatatttttttttaatgaaccccatgagaactaggtccaattagcacctcttgggtgggatagtttttttgggtgagttagatggaactagctccaatctaacccttatgtttggatactttagggctagttgaactcaaactagctcaaactaactctagcccaaggatccaaacagggccgatatttgtgactgaaattgtcatggccaaggagtagagctgccaagcacaagtacgtacataaaccaacgggacgacaatatccgacttgatgttcaaaagcatcttgctaacttaattagaaacttacagctagactaaagcacacacttagtaagttttttgggataacgtaaacattgacgaccacattgatgctcgacgaactcattgacaacacaagactgctgcagccctttcttcacccggagaacacaaaaagcattaatctgcaagtaacccaatacaaataaagataaaatctaatagaagcacccatgtaactgtttagcccatgaaaaggaatatgtgtacaaactcacgatcagcccagctatataaagaagagctatatcatatcccttcgaaaaaaagacgagctacatctcctgcgcataccaatgacaaaataaaataaaaccgttcagcctttttcataggaatttgttgggtggatgagtagaatgatgtggctgatgtttgaaataacaagatggctactaatatattgctttcagtttatatgagacagccacctgggtatgaggtaaaaggcaaacatcattatgtgtgcaagcttgataagacattgtatggtttgaaacaagcacctagggcatggtactctaggctcagtacaaagcttacaaggcttggttttcagttatctaaggctgacacttcacttttcttctacaagaaaggcaatgttatcatttttatgctggtatgtgttgatgacattactgtggcaagttcatcacaggagacaacaaatgcactgctagagggtctgcaaaaagattttgctctaaaagatcttggtgacttgcattacttcttgggaatagaggtaaagaaggtaacaaatggtatacttcttagccaagaaaagtatgtatcagatatactggaaaagactggtatgatgagttgcaaagtttcaaacatgactctatccactactgaaaaattgtctaaaaaagcaggtgaactcttgggagacgaaggagccacaaaatagagaagtgtagtaggtgctttacaatatttaaaccttacatgacctgacatatgttttcagtaagcgaagtatgtcaattcttgcatcctcctacttctcagcattggactgctgtaaagagaattctgagatatctcagaggtaccatgagtacagggttaaaattcacaagatcaacctcaactatggtaactgctttttcagatgctgattgggcaggttgtcctgatgacagaaggtccacatgaggctttgttgttttctttggatcaaatcttatatcttggagtgcacgcaagcaagcaagtaactgtgtcaagatcaagtactaaagttgagtataaagcattagcaaatgccacatctgaattgatctgggttcaagccttacttgatgaacttggcgtttgtcattcttcggttgcatggctttggtgtgataatattggtgctacataccttttagcaaatccagtatttcatgcaagaacaaaacacattgaggttgactatcattttatacgtgaaagattagcaaaaaggctgcttgacatccggttcatccccactaatgaccaggtagcagatggattcaccaaagtcttgtcttggccaaaattggaagaattcaaatacaatctcaacttggtgaagttatgattgagagagtgtgtgttaaagtgtgtgtatacacacacacacacacatacggtgtgtatataaaccgtatacatgggtagataagggacgccattgtgtagttgtatctctggtagttaggtggtttagattgatctctctttgccctgtaagtaacaagcagcgcgtccctgcagaagcatacgcttaaaccgaacttcacacatgaaacaactcaaaagattcatgatatacttggcatcaccctgttttacttgtttttctgtgttattaattagcatgtgtgaatcccaatagccgaaaggttagtagtttcagggtgagcactactcacattggctgccagatattttccaaggagaaaatcaaacacctcaaatctcacaaactatattcacaaggccttactttttgaaccgctctggcacattagcacaccaaagcagagtcctcacatttcagctccatcccatccacgagcaatggctgcgcctagaagcataagcatgagatgaaaacggctagtctgttacatagatgaattaacaaatgcaggggaaaaaaactcacttgtgtattaatgttttatagatgactattggcaacagataaacaaaaatacatatgttgcttttttaaggaatatatttttagagcagtattgtatacatagctacctcatattaacgaataatttagctcataagaaattaagaaaaggatcaggaatttaactcatccttcatcatatcatatgaagaagagaaaataaaagcatgccaaaactcatacactgccagcaaatatttacttgggtattgaaggagaaatattagacatagcaaaacacatgaataggggtgtgttacaaaagtactcccttttgcaaagtgatctaagaggtcatatatttgtttacagaggtagtgctacttatgaaaaaaatgatagtcttgaataagtgacgaagaaaattattctaccagaatgtatggtttcgacggaagttcttttatgccattataagaaaaatagcaggattaaaagtacatgatagtataacatagctagttgaggttccaaatatgcctaatgagttgaaccatttgacatttgaatttacactattagtcatttcatctgatatttggttttgctatattttaaggatgaaagacaactatattaaaccataacgcaagataatgctatattaaatgataacatatactagacattacggctataagctcatttgtctagcgaaggtattcaagcatatatgtacaaaacataacaagattaggatgctcctacgatgttagtattaatgaagaaaagtcacctggcatatagaaactcttgaaagaataacagccatgaacatgaccggtttcataaattgtcttagaatgcattgacttaagatgaagcacgtagagaccggtgcccacatgtagaaatatatcatcagtatcctcagcatatctcaaatattaacttttataaggtaaaaactaacttttatacataatctcaaatattaacataaaaattaatttgtaacttaatctaatctaaatttttttttattttttataactctatttgctattataacttaatgtaatttgaatatttttataatttttacaacttgtgtcacattgggaagttcaaaaatgtacccaacatattataataagactgaaatacacacatatgaattcgtaattagactacgtgtcctcacacatggattcacaaatagggagacggagatggacatgctaccggtgcgtcgacgggcgacgacggcgggcttcggaacgacgacgggcgacgcgcgacgacaacaacgacgacgacaaggacggggacgacgggcgacgatgacgaagacgatgggctcggggcgacagtgacgacgacgacgatggactcggggattacggcgcgcgatgggctcggggcgatgacgggctcggggacgacagcgcgtgacgggctcggggacgacgacgggcgatgggctcggggcgacgacgggctcgggagtgacggcgacggcgacgacgggctcgtggaggaggcgcgggccggcggcgacgggctcgggtgcgacggcgatgatgaagagagaacggggggatggaagttggtggaaatttcgataactcccgcaatatacgaatacctttgtttccgtttggtggctcaaacaggaaccaaagcaacacttttgtcctggtttggtacaccaaccgggactaaaggtcaatttcgtcggcccaaaagggcaggaagaagagacctgtcgtatcggttggtggctacaaccgggaccaaagggtggcattggttcctgtttgagccaccaaccgggactacagatcaattttcatcagccaaaaagggcgggacgaagagacctttagtaccggttggtgggatgaaccgggacgaaaggtaggtattggtcccggttggggccaccaaccgggaccaaagtgtggtgctacccggggccaaaagtttagtcccacatcgctagccgagagaggtagcgagtggtttataagagctcctacgcacaacatctcgagctcctctcaattgtaatcttccgggcctaatctgtcactgcctgtgggcctactgggcctaccgcgggcctgaatcctggcccaataggctgattggctttctagtcgtattcaggccgtggtcgccgagtaggtggcattttttttaaagaaatctattttttatttttactttatttattttattttgttcctgcttactgtttttgggtttatacaaggtttatgaaaaacgtatatttccaaagaagtgagtgggagcactatagaatttctgataagtatatgtggttgacatattgttgatcgaaagtaatgtagaatttctgtaaagcataaaaggattgtttgaaatgagtttttcaaaggaaaacctggattgagctacttgaacattgagcatcaagaactatggagatagatcgaaacgcttaatagaactttcaacaaaatgcatgccttgacaagtttttgaaggagttcaaaatagatcagcaaataaggagttcttggctctgttgtaaggtgtgaatttgagtaagactcaaagcccgaccacggcagaagaaagagaaaggacgaaggtcgtcccctatgcattatccataggctatatagtatgctatgctgtgtaccgcacctgatggatgctttgccatgaatttgtcaaggggtacataagtgatccaggaatggattactggacagctgtcaaaagttatccttagtaactagtggactaaggaattttttctcgattatggaggtgttaaaagagtttgtcgtaaagggttacgccaatgcaagctttgacactaatccgaataactatgagtagtaaaccggattcgtatagtggagcagtcatttggaataattccaaatggtgcgtggtagcaacatctataggatgacatagagatttgtaaagcacacacggatatgaaaggttcagacctattgactgaaaacctctctcacaagcaagacatgatcgaaccccagaactgtatgggtgttaggtttattacaatcacatagtgatgtgaactagattattgactctagtgcaagtgggagactgttggaaatatgccctagaagcaataataaattggttattattatatttccttgttcattataatcgtttattatccatgctagaattgtattgactggaaactcaaatacatgtgtggatacatagacaacacactgtccctagtcagcctctagttgactagctcattgatcaaggatggtcaaggtttcttggccataggcaagtgttgtcacttgataacgggatcacatcattaggagaatgatgtgatgtacaagacccaaactataaacgtagcatatgatcgtgtcagtttattgctactgttttctgcatgtcaaagtatgtattcctatgaccatgagatcatacaactcaaggacaccggaggaataccttgtgtgtatcaaacgtcgcaacgtaactgggtgactataaagatgctctacaggtatctccgaaggtgttagttgagttagtatggatcaagactgggatttgtcactccatatgacggagaggtatctcggggcccactcggtaatacaacatcacaacaagccatgcaagcaatgtgactaatgagttagttatgagatcttgcattacgaaacgagtaaagagacttgccagtaacgagattgaactaggtatgaagataccgacgattgaatctcgggcaagtaacatacccaaggacaaagggaacaacatacgggattaattgaatccttggcactgaggttcaaacgataagatcttcgtagaatatgtaggagacaatatggacatccaggtcccgctattgaatattgaccgaagagtctctcaggtcatgtctacatagttctcgaacgcgcagggtctgcacacttaaggttcggtgacgttttggtatagttgagttataggtgttggtaaccgaaagttgtttggagtctcggatgagatccatgatgtcacgaggtgctccgaaatggtccggaggtaaagattgatatatagaaagtcctgttttggtcaccggaaaagtttcgggctcgtcggtagtataccgggagtgccgggagggttgccagggaccatcgggaggggtgtcatgccccaagaggcctcaggggctgtgggaagagataaactagcccctaataggctggaataagtccgcactaaggcccataaggtttgagaaggaaaaaacacaaggtggaaagagtttccaagtgggaaggagaaatcctactccaaataggattggagtaggactccacgtatctcccttgtgcaagggaagagaaggcagccctagggcgcagccctctccctcctctccttctatatatactaagggttttgagggtttttgcacgatagaaaataaccacatgttgccctctctctctagatctgtttatcctctagtttcagcggtgcttaggcaaagccctattggaatagcaccaccaccaccaccactacgccgccgtgctggagaactcatctacctctccgccccctcttgctggatcaagaaggtggagattgtcatcgagctgtatgcgtgctgaacgcggaggtgtcgtccgttcggcactagatcgggaaggattgtgatgaagattgagggacggatcatgatgagattgtgggacagatcgtgatgagatcacggcacggatcatgatgagatcgcggcacggatcgtgggacgacggcgattttgaatcgcggaaacgttccactacatcagccgcattatatacgcttccgcttagcgatctacaagggtatgtagattcactctcccctctcatagatgatcatcaccatggataggtattacgtgtgcgtaggaaattttttgtttcccatgctacgttttccaacacagtggtcccggttcgtgtgcggaaccgagaccaaaggggtcatacgaaccgggaccaatggcccacgaggcccggccggcgccctggcctcacgaaccgggaccgatgccccatgggtcccggttcgtgagtgaaccaggactaatgagatttatcaagtggaccaaagccctcttaattttctactagtggatgcctctgggtagtcccattttgatgtgtggacatgtgtatatgtaaagtatatgcaatatatcatattttcatgatttaagagaaatgctaaataaggctttgctcccctatagcctttcattgcttttgatcgagccgtcgctaaatggcatagcccgatatttaaaacattggaaatatttgacctggcgttatggaaaatgcttttagttagtcctgtacagaaaaagcttccacttaatagtttatttcagtatttttcagtcgggtgtggtttaaatgatgttcattataaattgtaatctgcaggcactcggaaccaagggaaatatcaatgcgtctgcccatttaatctgcacgtggtttgaagatattctggggagttagtttcatattaggaggggtttagcgaagtatttcaactagaactagataaaaataaggaattggatcagccttagtttttcatttttgcaaaaacatgccttgctaactgtaaaaccaatatctccaatgaatgaggtgccatgtgtctataatgtccgagagggaactaacgatccgaaatttctggagagatatcctggggcgattatgaggcgacaggaggggagtgccctaagatgggctccccgccggccgacaggggacttggcctcgtcggcggacgtttctgggcgctgcctgagcctgacgacgacgacgacctacagaacgcaccagcggcctctccaaccccttccgacatcgtatgcgaatccattctggtgggatactcggaggagcaggtcgcagaatccatagatgggttcgtccctgattccgatcctgcctgggaagggctgtcggctaatgacgaagacagggtcgaggtgctccgacgcgtcgtgcaccggcgaacgtcgactaccgctataagaccatggaagggacctctaccgaaggtacgtcttccggctcttacgttagcagatttttttcattcttgtaaacaagcaccgctgagaaaaccacgtcgaccggcggcgacatctcgaccggcggcacatgtttcagataaaacggatcttgggattcgtgaggccaaagagtctcgtctgaattcaattgtatgccaacatgggctggactctaaatctgatgacttacgaacgcctgggtatatggcccagtacgactcttaccgagattgaggaggttaccgacgtggacaaggaaggtctattgtcggctgattctccaacagcttataaagattgtggggtaagatcgggaataactggagggagcaaggcgattctaggtttcccgtcacgatctggcacagctaggagtattcctgcgttggttactatggcggcgaggcaagctgtaccctctgcgcccgcggcaagtgcgtccgctggggcagcgcctgtggtgactgcacctgcggcggcagggcctgttgctagggtagctgctacagcggtggcaggcgcgcgggagcctaaggtgcctgatggtcagggacgcgggtctgctggtgctggaagaggtgcccctggagcttataatcggggtggcggaggtggttttcatggacacagaggctatgcaaaccattggaacgattttgccggtggatccggtggtggtcggtccaattggatggccggaggaggtcctgggggaggagccaatcaatggcgccgtcccttccagccaccccaaggcaacttttgttggaattatgccctagaggcaataataaa

SEQ ID NO: 10 - protein, 598 aa, Hordeum vulgare
MPTALKALTLLFVLAVLAPDQSEGRHHRRDCPSFSCGPHGNVSSPFRRASDPPGCGYKSYELVCSDTKAIIRIRSAPYYVSGINYNDATFWVLDADLDLYSSCPLPQWNPPSHLTDVNQGMVELSPVSYTEACFVDNEMYVLVACLSTNDSFVYVLTGYGVCGIEYLELSCGYLSMTRLTWEVDVQQPLYNVSYADVVKSMRSGFAIRFPYPITLRFDCTHSSISWSSLGVCWLQYFILAPLSVLIFLAYKYWQARITIDAVERFLRMQQVLGPRRYAYTDITAITGHFRDKLGQGGYGSVYKGVLLPGNVHVAIKMLVSSSNCNGEDFISQVSTIGRIHHVNVVRLVGFCPEELRQALVYEYMSGGSLDKYIFSVERSFSWDKLAEIALGIARGIDYLHQGCEMQILHFDIKPHNILLDSNFVPKIADFGLAKLYPRDNSFVPSRALRGTIGYIAPEMISRSFGVISSKSDVYSFGMLLLEMAGGRRNADPNAANSSQSYYPSWVYDKLTAPVVDAICPVASMHELEKKLCIVGLWCIQMKSHDRPTMSEVIDMLEGGFDGLQMPSRPFFCDDDHTAVPDSYPLLSELTEISEEDDE

SEQ ID NO: 11 - DNA, 2580 bp, Hordeum vulgare
atggatttgcttaaatttcttctgacaccgctgctgctatctcttctaacccatcaaacctatgtgggcgcagcatcggatgatgaaggtttctccaaacaatgttcacctcacaggtgcagcaaacatggccctgagatccggttcccgttcgggctatcatcatgtggcgcacctggcatgcagttatcatgctcccgggatgacataatcctggatcaccctgttcttggctcctgcaaagtgaccgagatctactacagacaccgtgtcatcaacgtcgccccgcttgcggaacctgcgatgcagtgcccacttcagaggctcatctcaacaaatttagcaactgatgtgtacaaactacctccatcacgaacgaccctggtacgttgttcaagcggattcatacctgcagatccatacagcatagctggcccagctgcttgtctcagtaacaacgcaacccaattctggtatttggcggcgtcagcttatgcatacatatatgatctaccacgagactgtgaggctgtttctagaggcattccagtaccctacagctatgatatacatggcccaaacttacaagacgatcttgccttcaacaaaaaagcgagtgcagtcatcagttttggtgaaacaacattcacttggtacctcagtaaccttaccgatgtctgccaacagtgtgaacgtgaaggtcgacactgtggattcagttcacgaagccgtcaagcattctgccagccccatggtattctttctttcccttggtgtgtcaatttattttggaacaacaattcaatctcttatattccggtgttaactgttttaaatcttccaataggattttcaccgatagttcaattatttaacaaacaaaaatgtgatgcaaatcatagaaatggttaagttgttcaagcacatacagtctgtaggttcacatccttgaaagttcccaccaaaaaacatccgttaagagatgcaagtgccaaattatatgggaaaacctatggtggtactaatttgtaaaattctcggcacataaaaagtaagagatgaaccttaattgatcatttttcatattgctttagtatttgatatctgaatatgaataaataagtgtacaactataacataacaaagaataattaatcgatattacaccatatgtctaaggtccaatagacacgcaccaacgaatcaggaattagttgttccaaacatactttgtagaagtaatgaagtagtcttctgttttcatctacattcaaattaaatcatggaaaaaactaatgatattcttcctacatgcaaatgcaggtacccatgtcgtcccaattgcgggtaatgttgcaactcttttccttttgctcattctaaatgaaattcttgttgcttaccactataactcctttgtgttttcccattacaataagagtcgggaatgcatggcctcttatctgaatgcaattagctaatttcctttattttttctaatcagcagcctcatctgtagctgcatttgtagttttttcatcgatggtggccattgtgatatatttctccttgaagtcaaggtacaatgaagagataaatatgaaggttgaaatgtttctcaaggcatatggcacatcgaaacctacaagatacaccttccgtgatgttaagaagatagcaagaaggttcaaggataaactcggtcagggtggattcggaagtgtgtacaaaggcgagctaccaaatggagtacccgtggcagtcaagatgctagagagctctacaggagagggggaggacttcatcaatgaagttgcaaccattggactgatccaccatgcaaatatcgtacgtctcttgggcttttgctccgaaggaatgagacgggcccttatttatgaattcatgcctaacgagtcactggagaaatacatattcccacaagttccgaatatctctcgacagctcctagcacccaacaaaatgctagatattgctttaggcattgccagaggaatggaatacctgcatcaaggctgcaaccagcgcatcctccactttgacatcaagccacataacatcctgctggactacaacttcaatccaaagatctcagactttggccttgcaaagctgtgtgcaagggaccaaagcatcattaccttgactgcagccagaggcacaatgggatacatcgcaccagaggtatattctcggaactttggaggggtgtcttacaagtcagacgtgtacagtttcggcatgctggtgctagaaatggtgagtggaaggaggaactcagacccaagtgttgagtaccaggacgaggtatatctccctgaatgggtctacgagagagtaatcagtggacatgaatgggagcttacttcagaaatgacaggagaagaaaaagaaaagatgaggcagctgactattgtggccctgtggtgtatccagtggaacccgaagaatcggccatcaatgacaaaggtggtaaacatgttaactgggaggttgcagaacctacagattccccctaagccctttgtgtcgtctgaaaatcatcatcctagaacataa

SEQ ID NO: 12 - DNA, 1542 bp, Hordeum vulgare
atggatttgcttaaatttcttctgacaccgctgctgctatctcttctaacccatcaaacctatgtgggcgcagcatcggatgatgaaggtttctccaaacaatgttcacctcacaggtgcagcaaacatggccctgagatccggttcccgttcgggctatcatcatgtggcgcacctggcatgcagttatcatgctcccgggatgacataatcctggatcaccctgttcttggctcctgcaaagtgaccgagatctactacagacaccgtgtcatcaacgtcgccccgcttgcggaacctgcgatgcagtgcccacttcagaggctcatctcaacaaatttagcaactgatgtgtacaaactacctccatcacgaacgaccctggtgaaggtcgacactgtggattcagttcacgaagccgtcaagcattctgccagccccatggtacccatgtcgtcccaattgcgggcctcatctgtagctgcatttgtagttttttcatcgatggtggccattgtgatatatttctccttgaagtcaaggtacaatgaagagataaatatgaaggttgaaatgtttctcaaggcatatggcacatcgaaacctacaagatacaccttccgtgatgttaagaagatagcaagaaggttcaaggataaactcggtcagggtggattcggaagtgtgtacaaaggcgagctaccaaatggagtacccgtggcagtcaagatgctagagagctctacaggagagggggaggacttcatcaatgaagttgcaaccattggactgatccaccatgcaaatatcgtacgtctcttgggcttttgctccgaaggaatgagacgggcccttatttatgaattcatgcctaacgagtcactggagaaatacatattcccacaagttccgaatatctctcgacagctcctagcacccaacaaaatgctagatattgctttaggcattgccagaggaatggaatacctgcatcaaggctgcaaccagcgcatcctccactttgacatcaagccacataacatcctgctggactacaacttcaatccaaagatctcagactttggccttgcaaagctgtgtgcaagggaccaaagcatcattaccttgactgcagccagaggcacaatgggatacatcgcaccagaggtatattctcggaactttggaggggtgtcttacaagtcagacgtgtacagtttcggcatgctggtgctagaaatggtgagtggaaggaggaactcagacccaagtgttgagtaccaggacgaggtatatctccctgaatgggtctacgagagagtaatcagtggacatgaatgggagcttacttcagaaatgacaggagaagaaaaagaaaagatgaggcagctgactattgtggccctgtggtgtatccagtggaacccgaagaatcggccatcaatgacaaaggtggtaaacatgttaactgggaggttgcagaacctacagattccccctaagccctttgtgtcgtctgaaaatcatcatcctagaacataa

SEQ ID NO: 13 - DNA, 20422 bp, Hordeum vulgare
ggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgggctcagtgttcccatgcctccagcgagatttgtcatgggcagatagtacggctgcctccactgttcccacgtcgagtgcagtaatggagtgggagtttgacggtccgatactatgctgggtgatggatcagaccaggggtaggtcagcggcgtggccgccgacgctcgtacctgccaggcactccgatacagtacctttgctgacgcgtagccacctttaatcgtagagtctcgtcatgacctacgacctgatgtgacttaaggtccccggccggctagcctgcttgggcatggccgcctcgtttctagccggctggcttggccaaggcggtcaagaagaggtagccgtctcgtcctccagccggtaggtgcagtctagccggccagaggacttgggccttggggaattatacttggtcattccttttgggcaggagatgaaggagcaggctcgatgagcctaccccggggttatcccccgacacttgggctcagtgtatggagatgatgatgattgcagaggaattcgtgtcgactgcacttttatcacttttggtaaggttaagttcttgtaaccatgaatgtgattgtttacctgcagtccagtgtcgcatatggaccaaactagttgacagcagcagcctcaccgtcagctcaaggtgagaaaaattaagctgatttcttgctgtcacacgcattgtgatttaatcgacggcaattccaacggtataaaaatcatccaaataactttgaagttcagcagaaaactactgtacaagccaagaatgcaacctcctgccgtatgcgtttctgcaattacgagtgtgattgacaagcactgcaaggacgacgagtcctctgcctcatatgaaccactataaataactggacagcagcagcaacctcgctctcagggcaaaaaaaagtccagcatctgtcatccgaatttctacagcaataatttcagacatgtaaacgtcgaaacgacttgtagaccaagaacaaattgctaatgcgttaatttcagccatataaataacattcaaatgacttgaaagtccaggaaaaaagagagctaaaaagctaagaatgtgacctcctgcgtatgggttcttgcgattaccagtaccgctgtagactcgctgccgtatgtgtcccgcatcttcggcagagaatcatgaatgcgagtgcagacttgctctgaaaattcagagcctggagcgaaaggtgggaggggagggtgcgcgctgtcctctgggagctctatacgacgccttgtgcacccgactgccaaggaaacgcacaaaagactcagacctgggctggcccggtagccattttactgtagcaccagatcgataatacagtaaaaaaacacatgcgagaaaatgcttctgtagctgctgcatattgtaaaagacgaaccttttcaaaaattacaacattttaaattgaatatgtttctgaaaacatgatgatttttcaaatatgcgaaccttttttcaaaaaacgttgaacactttttggtttgggaaaagattacgtttcaccggctgatctgattgatccatccgccgcctgctcgtccgttagatctcgcctcgatcacgaaccatttcccgaaactggctcgttgtttcagaaactaaagcgttgattcatgacgggggctgaagctggagcactccaggttcctcgtcgtggctactctgctaccagggacgagcttgccggagtccggcgaaccgccaccgccatcggtcctcgccagctcgccggatccccacctcaccgacgagatccagcccctcgccgggtccccacctcaccggatcgccagatccccacctcgcgcggtgaacgaggaggtttgcggcgcggacgccggcaagcttccgccgccgggagcttctctgcctcgctggagatgcgtccctgctcgcacgcccgactctccgttaagcatggatcccggctgcaacatgacttttgttgcaaaaattcctgaaactcgaccgttgttgcaaaaaattcttttgtaaaaatatttatgttacagaaagatgaagaaaaataataattctgcaacaagcaaattgtttctgaaagtcgaccgttgtttcaggaattaacggatccaaagtttatttcttgcaataaatcgaaagttttcgcaactcgagcgtcgtttcagaaaataactgctgaccatgcgacgggaatgtgcggtgttagatcggcatccgacggctagctaggtgacgaataatttttaaaaatcatccggcggacgcgtagcgttccccttttgatttctggaacattttccgaattttttattatatgttgaaaataggaaacattgtttcgaaagcgcgaacatttttcagaaatacgaacatttgcaaacatttttacaaattccggaacattttttcaaaagccggaatacttttgaaattccggaacatttttatgaaaaatacaaaacaattttttcgtaacatttttctgaaaatccgaaactatttataaatattcggaacatttttcaaattctgaaacatcttctaaaatgccggattttttttaaattacggaacattttcttgaaaaatacgaaacatttttataaaattctggtacattttttcaaattccagaacatttttctaaaatccggaacgtttttttcaaaattctggaacattttttcaaattccagaacatttttcggaaaatacgaaacctttgtataaaaattccggaacattgtttgaaattctgaaacatttattcaaattctggaacattttttcaaattatgaaacattgtttcaatttatggaacaatttgttaaattttggaacatttttacaaattccgaaacattttttcaaattctggaacattttttcaaattccaaaacattttttgaaaatccaggacttttttgaaacagaaacagaaagaaagaaatataaaagaatgaagaaaaaagaaactgatttttttattatccagaacattttttgaaacaaaaacagaaaagaaaaacagaaaaagaaaactaaaaaaacaggttcagtgaacattatagaagttttccaaaaccggaaaaaaccgactaaaaaccctagaagattcccaaaaccggaaccacctaaacatgttaacgggccggcccatgtaatcactcctgtgtttgccctgttctgtgcgtttgcccgacagtttgacgcaaagagcatgaaataggatattccctgtcctcattcctagtgcacgagtcatgttatagaaggtcccgttacatcgtccgtgacacattttagtttggtcagtttatagcttctgcctcacaagatgcccaaattacaacaccacaaccattgtgagacctcctccccatcaccttaagcgtcaaggatggtattcaccgctcatgtgcatccccacttggactagcacatgcaaaggaggcgagcgctctctaaggacacactgttttcccggttggctgctcatgattgaccagtccagataggattttccaaaaagtagccagacaatttgtttacaacaaggtgatgggttctagggaaaatcctatatgccgctcactgcgtccaattgttgacgcttcgcacagggtgatgggtccaaaacaggaacactttgtgaaaacacgggcattttcaagacatcaggacttctttttaaaaatgtgaacaattaaaaaaatcaagaattttttcaataaaaaatatggacattcattaaaaagaaaataaagaaacaaaaacgaatatagaaaatagaaaaccaaacaaaaccggtaaaaactgaacatgaaccatgtaggaagttcttaaacacgtgactcgattgaggctaaataggctgatccatctctcggttattcaccttattaaaacgtcaaataatatggatcctagggagtaaatttttttaaaaacgtaaaatgctactcccttagatccatagaaatttgaaatcaacgacaattaatatggattatagcagtagcatcttttgaaattataggtaatttatttaaaatttcaacatttttttaaaatataaacatttaaaaaaactcccccaaggaacattctctgaaaacacgacattttaaaaaatcaagacttatatatgaaaagttaaccgtttaaaaaatctaacattttgaaaaattcaagacatgtatatgaaaagtgaacagttaaaaatctaatattttccctaatttcataacatttaagaaaaaggaaagagaaaaagataaaaaccaaaatataaagcaaaaaaccgaacatgaaccgtctagaaagttcctaaaaccaggattctgtagagcacgtagctgctatggctcgatgcatctctcggtcattcaccttcctgaaatgaggaatgttttgttgcagaatgcatcaaataggtttcatcattctttcgtctttcatctgctttcgaatttccctttcgtcagggttaggtggagcactacagccgaaggccatgctcacatagagggcctatttggtgcaccgagcaattggtgctacttgtgcactggaccccacacaattgatgctcgccatgatggtgataagataaaaaaaacttttatttactttgtaggggcgcacaaccgcgatatggtgccacataatttgaccacgagcatgacaaaaaacaggagacatatgaatccaggcgtggatcggaggttacgttccgaacgtacaaccccctacacgatttgggtcagcccgttgggcgccatttgttacgttctttattttctattatcctcgttttttgttttaccattaaatttatatataataattttattttgagaaaattttaaaatattcacaaaaacattacaaagccataaaatgttcgtaacttcaaaagtattgtttattatttcaaaaactattcatgaatttcacagaatgttcacagagttcaaaacttgttcaattttttaaaaatggtcacaaattcaaaacaatcatggttttaaaaaaatgtttgtttcaaaacaaaatataaattaaaaaaatatgttgacaaacttcatagagtttctgaatataaaaaatgttcacaatttaagaaatgtgcaggaatttcaataaaatctggaagttaggaaggttgttaccaaagttcaaaaaaaacttgcctatatttaaaatgttccaaaattaaataattgttaacaattaaaacatatcttgaattttaaaaaatatttaagaatttgaaaattattctcttatattgacaaacgtgaatttaaatttgattgcaaattttataaatcatgcacaattttcaaagaaatcactaattttaaaaattgtttcaaggaaggatctagaagtttccaaaaccagtgggaaaaaagcagaatgggccatcccatgcagaggacagggcgtgtgtaggttatacggtagattgcttttttttatttaccacaatacaaacatagatgttcatacatatgtatatacactcaccttaacgaacgcacatgttattcttataagcatctttgaaagactaagccgacacaatatatcaagatctactagagagacacttatggttagttggttgatatccagataccttcatgggaaataagaaacacccaaaaacagttgatcagtttgtattattgcgtgtgcaagtctaccacaatgcccaaccctagttcttatcgatacatcattcttatggtgaaaacaactcaatggttcgtgcgtgcaaacaaggcccgcccgttttacacggctcgtcccccttgcctctccccatacctcaacacgttttggtcctcgacgatagatgacctctcaagtggctgcatcattaaaaagtgctacggcaattctttgtacttaggaattatcacacctgttgcatgccacaccatgtgatcgatgccgatcaaacaacagaacgtgcgactgatgttgttgatcgatgaaagcgtttttcaaataattggtggttggatggttaggagaacagaaatattcatgtcccgtgagagttcaagttctaggcttgaagttggtgctcgcattatttttgaatttatttttggcttctgtcagtgttcattcagtgaaaggagacatcccgtcgaggcgagacacatgtgatgactttgtaaagtctgaagatgctttgtcgactcagtctctcggagatgctcaaagaagtagagtatgcgtgcgtgcgtgcgtgtgtgcatgcataacgacaagtgtatgctcatgtatgcaagcgacttcgatcgtattgtgttaaaaaaaatcaattgcaaagtaaagacagtgacgaataaaaatccccttttgcaggggagcccaactctgcccgtttataacccttgtggcggacagtgatgaatattgtgtttaaaaaatatttttcggacagaaagacgtatccacattttttgcaactcggacccttgtggctgccggcgagcgattcccgccctaattctccggcgtctaagcgagctcctcgtctttccttgggctccggcgaagcgcccccattcctctgctccggtgagtaatcccccttgctctcacttcgtttttagtgacacgattattttcaggatctgcgcattagaaagagcttagttacagaagcgtcgatccgcagaacaaagctccatgtgattccgatatgctgcaaatatctactagtacatggtgatgactggttattcatgcatcagctggcaggtgctagcacaggggaggggaaagatcggaacatgttcagtcgactgctgatatttgaaggaacagggaaatagatattctgtgatgttctcagcacaatggaaatgacaatcatgtttcctgaggatcccagtgtggaataatgcactgtaggaaatataaatcagcggagcgtcacaacaaggtaagcaacaaaccactgcatttctaactgttattatacagtccaaatgaagttctcatatggtcttgcaaatacctggtagtagtattttattacatggggacggacgattattcatacaatcaggtgctaataagtaatagagtggaaagaaggaaatataatgcacgctagtgctactgctatgtattgttgtgcaacacatggactaagtgcatgtcttcaaataatggcagcattctctgtgcttttatgttctaggatgatgattttcagacgacacaaagggcttagggggaatctgtaggttctgcaacctcccagttaacatgtttaccacctttgtcattgatggccgattcttcgggttccactggatacaccacagggccacaatagtcagctgcctcatcttttctttttcttctcctgtcatttctgaagtaagctcccattcatgtccactgattactctctcgtagacccattcagggagatatacctcgtcctggtactcaacacttgggtctgagttcctccttccactcaccatttctagcaccagcatgccgaaactgtacacgtctgacttgtaagacacccctccaaagttccgagaatatacctctggtgcgatgtatcccattgtgcctctggctgcagtcaaggtaatgatgctttggtcccttgcacacagctttgcaaggccaaagtctgagatctttggattgaagttgtagtccagcaggatgttatgtggcttgatgtcaaagtggaggatgcgctggttgcagccttgatgcaggtattccattcctctggcaatgcctaaagcaatatctagcattttgttgggtgctaggagctgtcgagagatattcggaacttgtgggaatatgtatttctccagtgactcgttaggcatgaattcataaataagggcccgtctcattccttcggagcaaaagcccaagagacgtacgatatttgcatggtggatcagtccaatggttgcaacttcattgatgaagtcctccccctctcctgtagagctctctagcatcttgactgccacgggtactccatttggtagctcgcctttgtacacacttccgaatccaccctgaccgagtttatccttgaaccttcttgctatcttcttaacatcacggaaggtgtatcttgtaggtttcgatgtgccatatgccttgagaaacatttcaaccttcatatttatctcttcattgtaccttgacttcaaggagaaatatatcacaatggccaccatcgatgaaaaaactacaaatgcagctacagatgaggctgctgattagaaaaaataaaggaaattagctaattgcattcagataagaggccatgcattcccgactcttattgtaatgggaaaacacaaaggagttatagtggtaagcaacaagaatttcatttagaatgagcaaaaggaaaagagttgcaacattacccgcaattgggacgacatgggtacctgcatttgcatgtaggaagaatatcattagttttttccatgatttaatttgaatgtagatgaaaacagaagactacttcattacttctacaaagtatgtttggaacaactaattcctgattcgttggtgcgtgtctattggaccttagacatatggtgtaatatcgattaattattctttgttatgttatagttgtacacttatttattcatattcagatatcaaatactaaagcaatatgaaaaatgatcaattaaggttcatctcttactttttatgtgccgagaattttacaaattagtaccaccataggttttcccatataatttggcacttgcatctcttaacggatgttttttggtgggaactttcaaggatgtgaacctacagactgtatgtgcttgaacaacttaaccatttctatgatttgcatcacatttttgtttgttaaataattgaactatcggtgaaaatcctattggaagatttaaaacagttaacaccggaatataagagattgaattgttgttccaaaataaattgacacaccaagggaaagaaagaataccatggggctggcagaatgcttgacggcttcgtgaactgaatccacagtgtcgaccttcacgttcacactgttggcagacatcggtaaggttactgaggtaccaagtgaatgttgtttcaccaaaactgatgactgcactcgcttttttgttgaaggcaagatcgtcttgtaagtttgggccatgtatatcatagctgtagggtactggaatgcctctagaaacagcctcacagtctcgtggtagatcatatatgtatgcataagctgacgccgccaaataccagaattgggttgcgttgttactgagacaagcagctgggccagctatgctgtatggatctgcaggtatgaatccgcttgaacaacgtaccagggtcgttcgtgatggaggtagtttgtacacatcagttgctaaatttgttgagatgagcctctgaagtgggcactgcatcgcaggttccgcaagcggggcgacgttgatgacacggtgtctgtagtagatctcggtcactttgcaggagccaagaacagggtgatccaggattatgtcatcccgggagcatgataactgcatgccaggtgcgccacatgatgatagcccgaacgggaaccggatctcagggccatgtttgctgcacctgtgaggtgaacattgtttggagaaaccttcatcatccgatgctgcgcccacataggtttgatgggttagaagagatagcagcagcggtgtcagaagaaatttaagcaaatccatgggatgtattgggtatgaagatggaagaagagaaagccaggagtattaaaaatcgcagttttctttaacctctggtgaggatgggggtggcgcttgtcttgaaagtaatgtgcctggccaggaattccagctgtcaccaaaacctctgtcataggtgttgtatggctcagaaattaagttccgtggttctattactgttttggaccatttatgccatttcaccacacatatatcctctagtgctccaataaaattccaatcctctaaggacatggtaaagcataaaaaatatgtgcatccatttagctttataattatcatttcggttcggaccaccagttgtttcttgttagtttccatgtagtacttcccttccttcctattgcattataccatttaattcctgataggtcgttttccattgctacatctccatgatgaacctgtcttaatactgccaagaagatcttcaaggctatttacaagttggccataaaaaagaaagagttaactggatatttgagttatgcaacaaaaaacaagaggaggagaggagaagagtacacggtactgtgagtggcgaatactactgttgttgttactagagccagaaccaattttccatgggccccattgctttgatgattaagatgctccatcactagtggggacgggcctttagccccggcccgtaaggggttttagtcccggttcaccaaccgggactaaaggggcgggactaaaggcctaacctttcgtcccggccctcttacaagccgggactaaaggtgctccacatgggcgcctcgtagcgccccaggggcaggccctttagtcccggttcgttacacggaccgggactaaagattttcagattttcagattttgctggtttttgggtttttttaatgaaattatttttgcgttttagggttttagggtttaggtggtcgggagattaacgtcatgcctcgtttggtgttcgggaattagttttcatataatttaaatagaaataattatgcatatatatatatataagattaacttatcttacaagcgagcatatatatacaattatatggagatctgaattatcgggactagagcctgtctattcgattacatggacgaacattagtaatggcccttagctacactaaatcgtcctttgagctctataacttccgtcctcagaaatcccgcaagctcctctgcaacagcaatcgcgcgttgctctggtaggaccttcgtcctcatggccgtgtgctatataagaagagaagatgaatatgaatatcaatcatgataacaaagaatgacgggtaaaaatagaggtgtgaatgttcattgcttacgtcgaatctgtgatccttgagctcagaggtaaacgtgcgaatggtctcgcaaacatagtatccgcatagatgcgttccccgtggctgctggtcacactttacgagaatagaatatatataatcaaaataataatctagcatcataaatgtattgaaaattaatagaagtatatcataatactacttacctgagccgctctaaaggtcagcttctcaggaaagttaccgggagtcacgcacttgaaccgcttccataccctgcccgacaaggataatgatttgctaagttttcattaattgatatatcagaaaatcatcgaaagagaccgatagagcgcaagaatgattaaaattacccttggagcatgtcctgcaggctttggaactgttccaagggtctcgataatgggtcgaaggcatcaactcttcccttatcaatttgaatgtccaacagaatccaatggaagctgcacatgtatatatatatatgtgtcagtaacttatcaattacacttataagtgaatggacacaacagagtaaagaccctcacctgaagttgtatggaaacagtatgtggtcacagaaattttgatctattagaaaccttagaaggttttcctccgtctccttgggtttatcagttagcgtcgctatatgtattttatctgggtcaataaacccaatatttaggatggtcttacttttacaatccagaatctccattctgcataatagaatacaagttatatatagacaatgaattgaaataactaaacaagttatatgtagacaacgaattgaaaaacttacacacaatagcaactcataagcgatttgtcgagggcgtcgccattgtacatctggaagagttcatcaaagttgatatggatttcttcggagcggccgtagtactcccgtgggacactcagcacgatcatcgttctcccattctttgattcacttaagtaccatttatgcaagtaacgcatatttgttgggagattatctttgctgaccaaaggctctcccatgacaaactatggcttaggggctaccacagccttgggtatcctgtcgtcttgggaagccagcaaatcttcaactgagataccacattcagccgccaactcctttgctctttcaaaagcttgcccctgcaccggagggggaacattctcggttaacaccttgaggggtgggatcgactgtttggcctgttgtccaagctgaggaacgtctgattttttcttgcttgtagttgaacttgatttgctcccacttgcacttgcacgtgagctgctctttttcacttccttctgcaatgtgcgtgtatagtcatcaggcttatagtgtaagtcatactgtgatggaagggttatgaagtcttttgcccatgctatttgtttctcggtgtattccgggcggggctcgggttccttctttttcatctgcgcatcatgttgttcctttgctatcctggcgttttcctcgggggtacgatcataaggtttgataggaagattagcatgaggtacctttgggagtggcgacagtttgcgctttggggagctccgtcgcttaaaaatcatcgacggagcgttcttgctggcacacttccgcttagtatcctgtgcgggcggcggcggagacggacgacccaagtccggcggcggtgatcgagatggactcgtgttgtgctggccgacgtcatgtggagggcttggaggtaatggaggtgtaggtgacctgcgacgactcggaggcggtgttgtccttggggtcgagcctggaagcttgatgtagttcttgtgcatcgtcgggcggggcctcagattcagccacgccgcttttcccttagatggggcgctggtaatatcaagtgcaggatcttcctggcgcggtactcctctaagctcatcaatctgcttttgttgctcgttaatcctggcaagcaactggttgaacttgtcattctcctcatcctgctgccgcttctttgctctctctcggcttctgtaagtgtcttggtctctggcaaacccaagccaccacgggtaagaaggaccgaagtctcgcgttcgtcctccatgttcgtcattgccgaggaccagtgtgagcaaatatttatctctatctgcagtgaactttctttttccctccttaatttctttcactatccttttccaattctccctgggtatcctaagaccgtcattgcagatgagctcccctgttttttcgtcgtacgacccaccatgcgcaaggaatcaatttcttgctcttaattcccactcatcacggagtggttcaggtaagatgcctttatctagcagatcttgctctttcttatcccactttgggatggcagtctcatagccccctggccccagcttgtggtgatatttcttcttgtcggcatttatcttgttcttttctgataatgtctgggcatcttctgactccttgtactcttgaaatgccttccagtgattcgcctgcttggctagatacccctcgaatactggcactttctttgtcttcagatagttttccatagcttcttcttccagctacggaacagttcggccatcttcttcagagtccattgcttgactttggccctcagtttgtctgcggcgtcttcattctcacattctggcaggttgaaatgtgacatgagatcattccaaagattatctttgtacctttcggcgacatagtcactatcggctgcccttttgcgcttgttccactcccgaacgctgatcgggacgtgatccctaacgagaactccgcattgcttcttgaatgtgtcagcaacattcttaggaagcttgggttcgcccgtaggaaatatcacctcaaaggtgtaatgcgtccgtgcatccaactttctagtcgggcctcgtttcgtagttttgctcgatgtggaggcctaagagggagaaacattcatcaaatgaatgtatatgtatacaatatagagctatctccaatatttttcacatattacaagtgattgtcgaacttcatatgtatccctcgccggactttattatttcttcaccggcttctccatcagtggagctatcaccttctccgtcattggacctatctcctgccccatcggcttcatcttcgtgtcgctcgacctccattccaacgtcctgctttagatatgacgacggagattcagctggttcaacgtcggggccgtctttgattatatcttcgagaagttcttcctcttcgaggtttttgatatgtggatccatagttctgcaaaaaacggattctcttaacctttgtaccaaaaaagaattattctatcaggaactccgttcgaaaacaacttaagtcccgggtcgtggctccacccgggactcctagatttttgcatagtattcaaagtaggagtacttttccaatttgagcattcaataagcaaaaccaaatcgtaaaataaagtagtattcaaattagcatgcattcaattataagctaatacatcatcacttttgtccgtacaccgtcgaatattatcactaatactcctcgaatactatcatacatatagcatcactgatacatctagaaccgtagcgcccgacgtgtatcggcgcgggcggtggacacccaaagagaaggaaccatcacaggatcatagctccagtgagatccccgaagaacctgccaggtatgctcgaacctgccctccaatgcaaccatgtagcgacggacgtgctcatcctcctcgctgacacggtgacgtaccacctccgcggtgtccggaagcctcggcaccctcactggcccacgcgaccgccaccaaacaaggatcgtgtcaacgacgggctggctcctcaccaacctacgccccccggaaggtagcaccttccaataccagccgggcggagcccagtcccagacagggcccctctgatcaagcaggtgtcctccgccgagtcgacgacgaggatgcgggatgcatcgtaaaatgaactaaaaaaataaactagttctattaattttcttgctaaaaataaactattaacacttaagcatatacaatagcaaaattaaactattaacacttaagcatatacaatagcaaaattaagcaataatctaagaaacactattaacacttaagcatatacactatacaatagcaaaattaagcatatacactatacaatagcaaaactaaatgacactatacaatatgtctcctcttcttctacttcttcttctacttctacttctcctcttcttgtagtacttctcctcttctcttcttctacttctcctctcctcctcttctaattttttctcttcttctacttctcctcttcttctatttttcctcttcttctcctctcctcctcttcttattctcctttttcttcttttcttctcctcctcttcttattttcatttttcctaatcttattttcttatttttattcatatttcttcttcttgtaaccctaacctaattctaaatattactaaccctaataatctagcacaaacctaataacaattggaattaaatgacattttttttctttttcttcctttcttctcctttttcttcctttcttctcctttttcttcctttcttccctttttctttttttcttctcatttttcttattttcttctcctttttcttcttttcttctcctttttcttcttttcttcttctatttttcctcttcttgtcctctcctcctcttcttttcttctcctttttcttattttcttctccttttccttcttttcttctactggccggagtgttggggaggagggggcggggggcttaccggccggaggtgtcgacggcgcgcggcgaggaggacggcgcgcgcgaggacgacggcggcggcgaggaggacggcaggcggcggcgagcaggacgtcgggcagcctgacggcgtcgtcgagttcgtcgctgtgccgcaccgggcaggagaacgagaggaagaagaagagaaatggacgatttgggttggaatttttcgaagtcccgcttatataggtggatctttagtcccggtgcgtggctcaaaccgggactaaagacaccctttagtcccgggtggagccacggcccgggactaaagcccctcgtttcccgcctcctcgcctgccgaaaaggggtctttagtcccggggcgtggctccaacggggactaaagggggtctttagtcccggggcgtggctccacccgggactaaagcccctcctcggctgcacgataagtttagtcccacctcgcccagcgagggggactaacacttgtttataagccccgtcgtagcttctccatcgagctcctctctaaagcaggcttacgggcctaaactcactgaaaattgaaactatattagaaattatagagaaaattcaatctaaattcaaatgagaatttagattgaattttctctatgatttcgaatatagtttcaattttattctattttcataattaataattttgactatccaaactattatctattttgttattttcaattaaaaactatgtttattaaaaattctttttgcatatttgagaatttgacaaaactatgataatgaaaagtgtttgaaattgaataaataatgcaaaactattttttattttcataattaattattttgactatccaaactattatctattttgttattttcaattaaaaactatgtttattaaaaattctttttgcatatttgagaatttgacaaaactatgataatgaaaagtgtttgaaattgaataaataatgcaaaactattttctattttcaaaagtaattattttgacttcgtgtacaaaacggacaatctctctcgaagtaggagcgtttcgaacgagaactcatctcttacaaagggatttcatttttttgaactagttgaccctgaaattgaaaagcactacaaatgaactctgaaaatgttgaaagttggcatgctatcatcatttcacccacatagcatgtgttaaaaagttgagagggctacgacaaaaactggatgcacttcgtatacaaaacggacaatctctctcgaagtatcagggtttcgaacgagaactcatctcttacaaaaggatttcatttttttgaacttatttgaactccatactttttgtgtgttcaaaatgcaccattctaaggcacatcacaaaatttcaacaatttctgacttcatttggtattcttcatgcatttacttattttttttgagctagttgaccctgaaattgaaaagcactacaaatgaactctgaaaatgttgaaagttggcatgctatcatcatttcacccacatagcatgtgttaaaaagttgagagggctacgacgaaaactggatgcacttcgtgtacaaaacgaacaatctctctcgaagtatcatggtttcgaacgagaactcatctcttacaaagggatttcatttcttttgaacttatttgaactccatactttttgtgtgttcaaaatgcaccattctaagacacatcaaaaaatttcaacaatttctgacttcatttggtattcttcgtgcatttacttatttttaaatgactgttttaatcaaaaacagatctgttacaaaagggatttcattttttaaacttatttgaactgaagactttttgtatatatatgtggtcgaaatgcatgataccataaagttagaaagggctaaaccattcaaaagtagcaaatgaagttagaaagggctaaaccattcaaatttggaaactataatgacacaaacagaaactagacatatataaattggtccaagaagtacatgatactagtccaaacagtacataataatagctaccatagatatatatacaagtgttcgacgttcagaacactactcgtctgaatcgtctaaatcagaatgtccaccttcctcgaggtgacactgacatagttgacgactcgaatcttgcggtacaactcgtatgaaacgtatgcatcttttgctgcatactcaatgttgataggatcaagtgggcccttctcccaaagtttgtgctgagactttgggaaactggtcttcatatcaccatatccctcgtcgatcaaggcaactaccatatgagccatcgaagtcctgtcatgtcgaagcctgaataccgtttggagatcaacaaggcaatcagctggtatctcaataccgaagctgtgcctcatcttcagcttgtcgttccttatgtcaatggtagcaaaagtgatgccgctgcgaaggaagtccatgagttctggacaatgcttgtcactcctgtaacagaaacaaattaaaatggaacaaatgttacatactgctgcaataaggaaacatgtttcactacaactaaagagaaaattatctttaggattcaaatagaacatatgcaatggaacctagagagatcactatagctatccaatggaacctagagagatcactagctatatgcaattttcatgactatgacatattatattgctatccaatggaacctagagagatcactagctatattcaattttcataaccttggatcaaagaacaccgcataaaattgtatcactacaactatgatttcaatggaacatattgaaccaatcagatttttcagattgtggaacactattccaatcagattgtgttcccttcaactaatcaaaattgtttcactacaactatttgaagcgaaccaactatgattccaatggacatattaaacactagttgattctagtacgatttttcagattatggaacactattccaatcagattgtgttccccttcaactaatcaaaattgtttcactacaactatttgaagggaaccaactatgattccaatggaacatattaaacactagttgactctaatacgatttttcagattatggaacactattccaattagattgtgttccccttaaactaatcaaaattgtttcactacaactatttgaagggaaccaactatg

SEQ ID NO: 14 - protein, 513 aa, Hordeum vulgare
MDLLKFLLTPLLLSLLTHQTYVGAASDDEGFSKQCSPHRCSKHGPEIRFPFGLSSCGAPGMQLSCSRDDIILDHPVLGSCKVTEIYYRHRVINVAPLAEPAMQCPLQRLISTNLATDVYKLPPSRTTLVKVDTVDSVHEAVKHSASPMVPMSSQLRASSVAAFVVFSSMVAIVIYFSLKSRYNEEINMKVEMFLKAYGTSKPTRYTFRDVKKIARRFKDKLGQGGFGSVYKGELPNGVPVAVKMLESSTGEGEDFINEVATIGLIHHANIVRLLGFCSEGMRRALIYEFMPNESLEKYIFPQVPNISRQLLAPNKMLDIALGIARGMEYLHQGCNQRILHFDIKPHNILLDYNFNPKISDFGLAKLCARDQSIITLTAARGTMGYIAPEVYSRNFGGVSYKSDVYSFGMLVLEMVSGRRNSDPSVEYQDEVYLPEWVYERVISGHEWELTSEMTGEEKEKMRQLTIVALWCIQWNPKNRPSMTKVVNMLTGRLQNLQIPPKPFVSSENHHPRT

SEQ ID NO: 15 - DNA, 2188 bp, Hordeum vulgare
atggcgatgcccactaccctcaaggtcttaactatcttgtatgtgcttgtacttcttgttgcatatcaggttgaagggcgaaatcatcggcctgattgtcctactttctcatgcggcccacttggatacgtatcatccccatttcgtcaggcaagtgatccacctgggtgtggtcatcaatcgtacgagctagtttgcagtgatactaaggctacaattcacatcgacaatgcaacatactatgtgtctgggatcaactacagtgattctaccttctggatcgtcgatgccgacatagatttatacaacagctgccctctaccgcggtggaattatgatcagcatccagctgctggtttcacgctgagaaaccggcaacacgacactcatcacgccatcaaagttgaattggcacctgcagcatataatcaggctatatttgttaattgttctcgggaagtgaaggacaacggtatgtacaggcctgttgcttgcttgagcaccagccattcttttgtttatgtgttaataactggtggcctagggagagggagtactatggcgaaccttgagccttgttgcggttatttggccatgactccattaggtggccaggaaattggcatgaagggcactgcaccgcttgagaatgcaagctatgcagatgtcgtaaaattcatgacacacggcttccgcgttaaatttcctttcagacttcgccaatctggagacttcttgggatgtctgatggatgatttggcgccgtgagtttgagatcttgatacctcgtacttgtaagctctgcttttgtttttgcacatgcacacctgatcattctacattttcgtttttttttgcagcacattcactaaagattggattctaacaattctttcgattgatgtaagtttcatgggttgcgcagctaaagcagttccagaaccatttgatatttgcttgtatataattatggcgtcattagcatatgccaggcttattgctggtacgtgagaattacatattccagtatggtttactgcctgtgtctataaatcagatcacaaataaacctgttttgttttatgcagtgttctggaggtttgtattggtgcccctggtcgtattaatcttccttgcccacaagtattggaaaacaaggatcacgatggatgctgttgagaagttcctccagatgcagcaaatgatcggcccgtcaaggtacgcctatacagacatcaccgcagtcaccagccatttcagagataagctgggtcagggaggctatggctccgtgtacaagggtgtgctgctcccaggcaatgtccatgtcggggtcaagatgctagagggtaactccaactgcaatggagaagatttcattagtgaggtttcaaccattggcaggatccaccatgtcaatgtggtgcgtttagttggcttctgctcggaggaaatgagaagggcgcttgtctacgagtacatgcctcgaggttctctggacaaatacatcttctcggctgagaagactttctcctgggacaagctcaacgagatcgctttgggcattgcgaggggtatcgactacctgcaccagggttgcgagatgcagattctccactttgacatcaagccgcacaacatcttgctagacagcaatttcatccctaaagttgctgatttcggtctcgctaaactatacccaagggacaaaagttttgtgccatcgagagccctacggggaacaattgggtacatagctcctgagatgatatctcgtagctttggtgtcatatcgagcaagtctgatgtttatagctttgggatgctcctgctagagatggccggaggaagaaggaatgctgatccaaatgcggcaaacacgagccaagcgtactatccgtcgtgggtgtatgacaaactaactgctccagtggtggatgcgatatgtccagttgctagcatgcatgaattagagaggaagctgtgcatcgttggattatggtgcattcagatgaagtcacatgacaggccaacgatgagcgaggtcatagacatgttggaaggtggctttgatggcctgcagatgccttccaggccattcttctgcgacgacgatcacaccgctgtcccggattcttaccctttgttgtccgagctgacagagatctcagaggaggataatgagtag

SEQ ID NO: 16 - DNA, 1824 bp, Hordeum vulgare
atggcgatgcccactaccctcaaggtcttaactatcttgtatgtgcttgtacttcttgttgcatatcaggttgaagggcgaaatcatcggcctgattgtcctactttctcatgcggcccacttggatacgtatcatccccatttcgtcaggcaagtgatccacctgggtgtggtcatcaatcgtacgagctagtttgcagtgatactaaggctacaattcacatcgacaatgcaacatactatgtgtctgggatcaactacagtgattctaccttctggatcgtcgatgccgacatagatttatacaacagctgccctctaccgcggtggaattatgatcagcatccagctgctggtttcacgctgagaaaccggcaacacgacactcatcacgccatcaaagttgaattggcacctgcagcatataatcaggctatatttgttaattgttctcgggaagtgaaggacaacggtatgtacaggcctgttgcttgcttgagcaccagccattcttttgtttatgtgttaataactggtggcctagggagagggagtactatggcgaaccttgagccttgttgcggttatttggccatgactccattaggtggccaggaaattggcatgaagggcactgcaccgcttgagaatgcaagctatgcagatgtcgtaaaattcatgacacacggcttccgcgttaaatttcctttcagacttcgtgcagtgttctggaggtttgtattggtgcccctggtcgtattaatcttccttgcccacaagtattggaaaacaaggatcacgatggatgctgttgagaagttcctccagatgcagcaaatgatcggcccgtcaaggtacgcctatacagacatcaccgcagtcaccagccatttcagagataagctgggtcagggaggctatggctccgtgtacaagggtgtgctgctcccaggcaatgtccatgtcggggtcaagatgctagagggtaactccaactgcaatggagaagatttcattagtgaggtttcaaccattggcaggatccaccatgtcaatgtggtgcgtttagttggcttctgctcggaggaaatgagaagggcgcttgtctacgagtacatgcctcgaggttctctggacaaatacatcttctcggctgagaagactttctcctgggacaagctcaacgagatcgctttgggcattgcgaggggtatcgactacctgcaccagggttgcgagatgcagattctccactttgacatcaagccgcacaacatcttgctagacagcaatttcatccctaaagttgctgatttcggtctcgctaaactatacccaagggacaaaagttttgtgccatcgagagccctacggggaacaattgggtacatagctcctgagatgatatctcgtagctttggtgtcatatcgagcaagtctgatgtttatagctttgggatgctcctgctagagatggccggaggaagaaggaatgctgatccaaatgcggcaaacacgagccaagcgtactatccgtcgtgggtgtatgacaaactaactgctccagtggtggatgcgatatgtccagttgctagcatgcatgaattagagaggaagctgtgcatcgttggattatggtgcattcagatgaagtcacatgacaggccaacgatgagcgaggtcatagacatgttggaaggtggctttgatggcctgcagatgccttccaggccattcttctgcgacgacgatcacaccgctgtcccggattcttaccctttgttgtccgagctgacagagatctcagaggaggataatgagtag

SEQ ID NO: 17 - DNA, 6193 bp, Hordeum vulgare
atccgatgaggtagcagcgagggccaccggtgtagcgctaccaggagtgcctgagacgtgggagacgcggtagaggtcaccggagctattgcacctcacaaggaccaccttggtgcgaagatccttcatggaaaaaccaaaagggtcaaactccacagaacataaattgtcacgagtgaattgacgcacgaaaatgaggttttggacaatgttgggggagaggagaatgtgagtgaggtagaagggtcgagaagaaagacgggcgttaccaatggcgacgaccgggaggcgagccccattaccgacaataatgtggcgagaatgagacgctaataaaggatgagacttgatcagaatacctgggtccccggtcatgtgggaagaagcccctgtgtccatgatccactcggcatgaggggaaaaagtaggcccatatcttggtgcagcgtggtacatggtcgtcgcgtcccaagtgggaggtgagccggagtaggggacaggcggcgcggtcgactggaccaccgggtaggcatgcgtcggagcgccgggacgaggaccaaggatgccggccaagttgggcgggatccacggcgcgcggggcggtgggaacggcatcccgacaggggcaaagtaccccagccagggctgctggtgggagccgcgaccgccactggaggggcatcaccgcggccgcggccacggccgcgcccgcgtccaccaccagcacggttgccgccaccgcgaccggcgtagggagcgccgccttggttgtcgccgccgcgcccaccgccaggagggttgccaccgccacgaccacgatcgccacgatccccgcggtcgccgcatccgccgccgcgctcatcatggacagccatcacgtgcgcgccctcagcgcgcgcccgcttgtctagggtgagctccaaaagggcgaggcgcgagcgggcctgggcgaaggagggaagaggcacagtggactggatgatggcggcctgaagctcgaactttttcccgatgccatcgagcatctgcagcgtcaaggtacgatcagagacgggttcgcggacgtcggcgagggcggcggcgatgttctgcagacgacgacagtagtcgttgacggagaggtcgccgcgaacgcacgcccggaactcctatccaagatgcatcgcgagttccgcctcgttcgcgaggaagaactcgtagaggcgcttccagatggtgtaggcggtggagtcggccggggcgacgatgtcgaggagagccccgtccactgtggcaaagaaccacagcacgattgtgttgtcgtcgtcgcgccactgtgggttggagaggcggtcgaccgcaccgtcctagacatggttttcggcgtggtagcaggcgaggatgaggttgaggaagttgcgccaccggatgaagttcgtgccatcaagggccagcttgaaatcaacgacgtcggagatgctggaagggaggcgagagggtggtggagcagggagggtctcagccggggcagtagagagggagaaggcaccggggacgggtggggcagaggtgagatcgggagagagagtcgcgtgggggcgttccggggaggcagaggctgctacggcggcagcagtgccaggaggtggagagttggggtgagaagacatagatcggtagctctgattaccaagaagaattgtgaaacaatgcacacactttactcatcaagtgattacacatatatacgccaagggtcaccaccgatccctacatgcagggatatggaggaggtggtgccgcggtcatccccaggaccaggtatggtggttacagtggataagtacaaggcagcgtaatgcacaaggaacgtaacaactcaacaataaaagccctttatcaaaaaaaaaatatgcaacactagactgcaggtacacactcacattcatggttacaagaatttagccttaggaaaaatagataaaagtgtgaacaccatcgtcgacatgaattcctctgcaatcatcatcatctccatacactaagcccaagctttatcaatcacaagggttcatacttcatacaggacaggctgggtctgttcatacccactccaaaaatttcgagcatcatcagcagctgaagaaaacagagtactctgcattttttacatcatgctttttgtgtgtgtctttcaagaagataaatacgtttcaattaacagttggagatctacatcctctaactgctaaatattattacatctttccacataaaacttcacatgtctgggaaaacataatagtaggttatttcgtaagacttgagacattaaatcacacacacacacacacatatgctactcattatcctcctctgagatctctgtcagctcggacaacaaagggtaagaatccgggacagcggtgtgatcgtcgtcgcagaagaatggcctggaaggcatctgcaggccatcaaagccaccttccaacatgtctatgacctcgctcatcgttggcctgtcatgtgacttcatctgaatgcaccataatccaacgatgcacagcttcctctctaattcatgcatgctagcaactggacatatcgcatccaccactggagcagttagtttgtcatacacccacgacggatagtacgcttggctcgtgtttgccgcatttggatcagcattccttcttcctccggccatctctagcaggagcatcccaaagctataaacatcagacttgctcgatatgacaccaaagctacgagatatcatctcaggagctatgtacccaattgttccccgtagggctctcgatggcacaaaacttttgtcccttgggtatagtttagcgagaccgaaatcagcaactttagggatgaaattgctgtctagcaagatgttgtgcggcttgatgtcaaagtggagaatctgcatctcgcaaccctggtgcaggtagtcgatacccctcgcaatgcccaaagcgatctcgttgagcttgtcccaggagaaagtcttctcagccgagaagatgtatttgtccagagaacctcgaggcatgtactcgtagacaagcgcccttctcatttcctccgagcagaagccaactaaacgcaccacattgacatggtggatcctgccaatggttgaaacctcactaatgaaatcttctccattgcagttggagttaccctctagcatcttgaccccgacatggacattgcctgggagcagcacacccttgtacacggagccatagcctccctgacccagcttatctctgaaatggctggtgactgcggtgatgtctgtataggcgtaccttgacgggccgatcatttgctgcatctggaggaacttctcaacagcatccatcgtgatccttgttttccaatacttgtgggcaaggaagattaatacgaccaggggcaccaatacaaacctccagaacactgcataaaacaaaacaggtttatttgtgatctgatttatagacacaggcagtaaaccatactggaatatgtaattctcacgtaccagcaataagcctggcatatgctaatgacgccataattatatacaagcaaatatcaaatggttctggaactgctttagctgcgcaacccatgaaacttacatcaatcgaaagaattgttagaatccaatctttagtgaatgtgctgcaaaaaaaaacgaaaatgtagaatgatcaggtgtgcatgtgcaaaaacaaaagcagagcttacaagtacgaggtatcaagatctcaaactcacggcgccaaatcatccatcagacatcccaagaagtctccagattggcgaagtctgaaaggaaatttaacgcggaagccgtgtgtcatgaattttacgacatctgcatagcttgcattctcaagcggtgcagtgcccttcatgccaatttcctggccacctaatggagtcatggccaaataaccgcaacaaggctcaaggttcgccatagtactccctctccctaggccaccagttattaacacataaacaaaagaatggctggtgctcaagcaagcaacaggcctgtacataccgttgtccttcacttcccgagaacaattaacaaatatagcctgattatatgctgcaggtgccaattcaactttgatggcgtgatgagtgtcgtgttgccggtttctcagcgtgaaaccagcagctggatgctgatcataattccaccgcggtagagggcagctgttgtataaatctatgtcggcatcgacgatccagaaggtagaatcactgtagttgatcccagacacatagtatgttgcattgtcgatgtgaattgtagccttagtatcactgcaaactagctcgtacgattgatgaccacacccaggtggatcacttgcctgacgaaatggggatgatacgtatccaagtgggccgcatgagaaagtaggacaatcaggccgatgatttcgcccttcaacctgatatgcaacaagaagtacaagcacatacaagatagttaagaccttgagggtagtgggcatcgccatggcagacttgtacaaagaagcacagacgtgcagatgaatggctgaataagtgagtggctgccagagttaaaaatagaactccaaagaatggacattacatgccaactagtgcctctacatgaacgtgatgttgctgactaggagacttggctactagccgccaactaaagttggatacataccacctactggctcatctaagtgatggtgatgttcttgaccgatgacctcttggttaacagaagagtccaattgtgttactatagcatcctgtactggtccaatcctgtcctagtcatggtaagatggaatttggtgccttgagaaaatgacaaatatcagggactaaaaaatgaaaaataagaaaatatcattcatttgagatattgagattacctaaggcatgttttgcaaagatgaaaaactaaggcttatctaattcctcatttttatctagttctagttgaaatacttcgctaaacccctcctaatatgaaactaactccccagaatatcttcaaaccacgtgcagattaaatgggcagatgcattgatatttcccttggttccaagtgcctgcagattacaatttataatggacatcatttaaactacgcccgactgaaaaatactgaaataaactattaagtggaagttttttttgtacaggacaaactaaaagcattttccataacgccaggtcaattattttcaatgctttaaatagtgggctatgccatttagcagcgactcgatcagaagctatgaaaggctatagcggagctataccggatctaaaccttatttagcgtttctgttaaatcatgaaaaattgaaacagtatataatcaagcatcaaatatgatatatcactagtagaaaattgggatttagtctcggttccagaggggctttagtcccagttcttcaaccgggacaaaacaatcgggactaaaggcccaaacatttagtcccgattggcttatgaaccggacccaaaggatctccacgcggccgttgcgaggcgcccaggcaggaggacctttagtcccggttggtaacaccaaccgggacccaaaggcagtcacgcgtcagcagctcgtaggcgttgggttctttgtttttttaaatgaggggggcgtaggggttttggagggttttattaggggtttcatattgtgttagatagctactacatatagagagaagtgacatttctttctccgtgcttggtcgacggtacctactacatatagagagaacttgatgttagctagtaagtaaatgaaggaaccattaattacacaacatcgtcatgaacatatatagagtgaaatgacctctctttctccgtgcttggtcaaacaactcgacgctactacatatagtacaggatcatacatatatacaatatataatatctcttgcaatccctaactagctaatatctatcgcccaccaaaatcggtaagaaatcctgatgatattctccatctgatggtatgacatgctcaataaagaatcccgtcaattcctcttgaattgctcgtatgcaatccagggtcgcgctattcgtcaccctgggtgaggaatagttattcttcaccccctctatttttgttggaattatgccctagaggcaataataaatatagttattattataa

SEQ ID NO: 18 - protein, 607 aa, Hordeum vulgare
MAMPTTLKVLTILYVLVLLVAYQVEGRNHRPDCPTFSCGPLGYVSSPFRQASDPPGCGHQSYELVCSDTKATIHIDNATYYVSGINYSDSTFWIVDADIDLYNSCPLPRWNYDQHPAAGFTLRNRQHDTHHAIKVELAPAAYNQAIFVNCSREVKDNGMYRPVACLSTSHSFVYVLITGGLGRGSTMANLEPCCGYLAMTPLGGQEIGMKGTAPLENASYADVVKFMTHGFRVKFPFRLRAVFWRFVLVPLVVLIFLAHKYWKTRITMDAVEKFLQMQQMIGPSRYAYTDITAVTSHFRDKLGQGGYGSVYKGVLLPGNVHVGVKMLEGNSNCNGEDFISEVSTIGRIHHVNVVRLVGFCSEEMRRALVYEYMPRGSLDKYIFSAEKTFSWDKLNEIALGIARGIDYLHQGCEMQILHFDIKPHNILLDSNFIPKVADFGLAKLYPRDKSFVPSRALRGTIGYIAPEMISRSFGVISSKSDVYSFGMLLLEMAGGRRNADPNAANTSQAYYPSWVYDKLTAPVVDAICPVASMHELERKLCIVGLWCIQMKSHDRPTMSEVIDMLEGGFDGLQMPSRPFFCDDDHTAVPDSYPLLSELTEISEEDNE

SEQ ID NO: 19 - DNA, 3005 bp, Hordeum vulgare
atggcaatgcgcactgctctcaagatcttaactatctcgtctgtgcttgtagttcttgctgcagatcaggtcgaagggcggcatcatttgcctgattgtccttctttctcgtgtggccctctcagaaatgtgtcatctccatttcgtcaggcaagtgatccacctgggtgtggttatccatcttacgagctaatttgcagtggtacgaaggcttcaattcacatcgacgatgcaacatactatgtatcttccatcaactacagtagttcttccttctgggtcgtcgatgccgacatggatttatacaacagctgccctctacctcggtggaatcggcctccgttcagataccagaaccacatggaagtcgaattggaacccctttcatataataaggcttgctttcttacatgctctcgggaagtaaaggacaatggtatgtacatgcctgttgcttgccttagcacaaatcatacttatgtttatgtcttaactggttatggatcttattcaatggagtatctcgagccttcttgtggttacttggctagaattcctcggccttgggacagtccagggccgctagaaaatgcaagtcatgcagatgttgtgaaatccatgaggattggatttgctgttcgattccctttcagatatgacattggcatgagtagaacaaccatcaaagagtgcctaataagccagtttcggtgagttttgcctgtataagtgtcttatcccaaaaccgttaagcccctctttcattccttctatgtctcgttggatggtattgcagcgacttatcgcaggattgtagtgatggcgtcaagagttgtgttcatggcaatgcgcactgctctcaagatcttaactatctcgtctgtgcttgtagttcttgctgcagatcaggtcgaagggcggcatcatttgcctgattgtccttctttctcgtgtggccctctcagaaatgtgtcatctccatttcgtcaggcaagtgatccacctgggtgtggttatccatcttacgagctaatttgcagtggtacgaaggcttcaattcacatcgacgatgcaacatactatgtatcttccatcaactacagtagttcttccttctgggtcgtcgatgccgacatggatttatacaacagctgccctctacctcggtggaatcggcctccgttcagataccagaaccacatggaagtcgaattggaacccctttcatataataaggcttgctttcttacatgctctcgggaagtaaaggacaatggtatgtacatgcctgttgcttgccttagcacaaatcatacttatgtttatgtcttaactggttatggatcttattcaatggagtatctcgagccttcttgtggttacttggctagaattcctcggccttgggacagtccagggccgctagaaaatgcaagtcatgcagatgttgtgaaatccatgaggattggatttgctgttcgattccctttcagatatgacattggcatgagtagaacaaccatcaaagagtgcctaataagccagtttcggtgagttttgcctgtataagtgtcttatcccaaaaccgttaagcccctctttcattccttctatgtctcgttggatggtattgcagcgacttatcgcaggattgtagtgatggcgtcaagagttgtgttcggaaaattcttttggtcgatggagctttctgggagtgctcactgtatgcaactggatatgatgattatccctcagaaatcatagcattagtcttcttctctatgaagtggctagctggtaattccgaatgttctgatcttaattgcagtttcttcatcatcacatctacgcatgtgtgtacccacccactaaaactatataaacttgtttatgcagtgttatgcaggtttgtgttggcaccactggctgtgttgatcttccttgcgcacaagtattggaaaacgaggatcacaattgatgctgtcgagaagttcctccgaatgcaacaaatgctaggtccaatgaggtatgcctacactgacatcacagcaatcacaagccatttcagagataagctgggtcagggaggctacgggtctgtgttcaagggtgtgctattgccaggcaatgttcatgttgctgtcaagatgctagagggcaatcctaactgcaatggagaagatttcatcaatgaagtctccaccattggtaggatccatcacgttaacgtggtgcgcctcatgggattctgctcagaggaaatgaggagagcactggtttacgagtacatgcctcaaggttctctagacaagtacatcttctcatccgggaagagtttttcctgggacaagctcgccaagatagctttgggcattgcaagggggatcgactaccttcatcagggttgtgacatgcagattctgcactttgacatcaagccacacaacatccttctcgacaacaattttgtccccaaagttgctgattttggtcttgcgaaactatacccaagggacaacagctttgtgccatggagagccctacggggaacaattgggtacatagctcctaagatgatatctcgtagcttcggcgccatatcgagcaagtctgatgtttacagctttgggatgctgctgctagagatggccggaggaagaaggaatgctgatccaaatgcggcaaacacgagccaagcgtactatccgtcgtgggtgtatgaccggctaactcaacaagatcatgtgggtgagatatttgctcatgtttatactgagatgcatgagttggagaggaagttgtgcattgttggactatggtgcattcaaatgaggtgtcaagaccgaccgacaatgaatgatgtcatagagatgctggaaggtggcatccatagcttgcaaatgccttcgaggccattcttctgtgatgaagggcacatccatgccgaggatacttaccatttgtcgtccgagctgactgaaatctccgaggacatgagtgcttag

SEQ ID NO: 20 - DNA, 1974 bp, Hordeum vulgare
atggcaatgcgcactgctctcaagatcttaactatctcgtctgtgcttgtagttcttgctgcagatcaggtcgaagggcggcatcatttgcctgattgtccttctttctcgtgtggccctctcagaaatgtgtcatctccatttcgtcaggcaagtgatccacctgggtgtggttatccatcttacgagctaatttgcagtggtacgaaggcttcaattcacatcgacgatgcaacatactatgtatcttccatcaactacagtagttcttccttctgggtcgtcgatgccgacatggatttatacaacagctgccctctacctcggtggaatcggcctccgttcagataccagaaccacatggaagtcgaattggaacccctttcatataataaggcttgctttcttacatgctctcgggaagtaaaggacaatggtatgtacatgcctgttgcttgccttagcacaaatcatacttatgtttatgtcttaactggttatggatcttattcaatggagtatctcgagccttcttgtggttacttggctagaattcctcggccttgggacagtccagggccgctagaaaatgcaagtcatgcagatgttgtgaaatccatgaggattggatttgctgttcgattccctttcagatatgacattggcatgagtagaacaaccatcaaagagtgcctaataagccagagcgacttatcgcaggattgtagtgatggcgtcaagagttgtgttcggaaaattcttttggtcgatggagctttctgggagtgctcactgtatgcaactggatatgatgattatccctcagaaatcatagcattagtcttcttctctatgaagtggctagctttatgcaggtttgtgttggcaccactggctgtgttgatcttccttgcgcacaagtattggaaaacgaggatcacaattgatgctgtcgagaagttcctccgaatgcaacaaatgctaggtccaatgaggtatgcctacactgacatcacagcaatcacaagccatttcagagataagctgggtcagggaggctacgggtctgtgttcaagggtgtgctattgccaggcaatgttcatgttgctgtcaagatgctagagggcaatcctaactgcaatggagaagatttcatcaatgaagtctccaccattggtaggatccatcacgttaacgtggtgcgcctcatgggattctgctcagaggaaatgaggagagcactggtttacgagtacatgcctcaaggttctctagacaagtacatcttctcatccgggaagagtttttcctgggacaagctcgccaagatagctttgggcattgcaagggggatcgactaccttcatcagggttgtgacatgcagattctgcactttgacatcaagccacacaacatccttctcgacaacaattttgtccccaaagttgctgattttggtcttgcgaaactatacccaagggacaacagctttgtgccatggagagccctacggggaacaattgggtacatagctcctaagatgatatctcgtagcttcggcgccatatcgagcaagtctgatgtttacagctttgggatgctgctgctagagatggccggaggaagaaggaatgctgatccaaatgcggcaaacacgagccaagcgtactatccgtcgtgggtgtatgaccggctaactcaacaagatcatgtgggtgagatatttgctcatgtttatactgagatgcatgagttggagaggaagttgtgcattgttggactatggtgcattcaaatgaggtgtcaagaccgaccgacaatgaatgatgtcatagagatgctggaaggtggcatccatagcttgcaaatgccttcgaggccattcttctgtgatgaagggcacatccatgccgaggatacttaccatttgtcgtccgagctgactgaaatctccgaggacatgagtgcttag

SEQ ID NO: 21 - DNA, 10400 bp, Hordeum vulgare
ctattggatattgaccgaggagtctctcgggtcatgtctacatagttctggaacccgcagggtctgcacacttaaggttcgacgttgttttatgcgtatttgagttatatggttggttaccgaatgttgttcggagtcccggatgagatcacggacgtcacgagggtttccggaatggtccggaaacgaagattgatatataggatgacctcatttgattaccggaaggttttcggagttaccgggaatgtaccgggaatgacgaatgggttccgggaattcaccgggggggcaacccaccccggggaagcccataggccttgagggtggcacaccagcccttagtgggctggtgggacagcccaaaagggctctatgcgccaagcaaagaaaatcaagaggaaaaggaaaaaaaaggagggaagaagtgggaaggaagggggactccctcccaccaaaccaagtccaactcggtttggggggggagtcctccccccttggactcggccgacccccttggggctccttgagccccaaggcaaggtcccctccctcccacctatatatacggaggttttagggctgatttgagacgactttcccacggcagcccgaccacatacctccacggtttttcctctagatcgcgtttctgcggagctcgggcggagccctgctgagacaaggtcatcaccaacctccggagcgccgtcacgctgccggagaactcttctacctctccgtctctcttgctggatcaagaaggccgagatcatcgtcgagctgtacgtgtgctgaacgcggaggtgccgtccgttcggtactagatcgtgggactgatcgcgggattgttcgcggggcggatcgagggacgtgaggacgttccactacatcaaccgcgttcactaacgcttctgctgtacgatctacaagggtacgtagatcactcatcccctctcgtagatggacatcaccatgataggtcttcgtgcgcgtaggaaattttttgtttcccatgcgacgttccccaacaactttgtcgaaggaacccgtggatcgaatttccggcaaagaggcgggggcggagtacgcaactatggtcggaataattctgggccgatatggaaggaagtcacacaagaggacgggggggaagatgacaccccgccaacacagcaaataacgacggaggacagggaagtagcgcgagccaacaaggcggctcgcaaaaaagaaaaattgacatgttataggtgtgggatacccggacacttcgtagtggactgtaccacggatttgtgtgatatctgtcaaaagcctgggcatattgatgaggcatgcccattacttttagctccaaagcctgtcatgaacatatatggggtttgccatagcaaactgatgttttttgagacaccaggttccacttcagttctaactccacctcgcctagagagctcaagaacgggtctcgtcaaagttacgaatggggagttgactgctgagcaagtctctcaacagatgagaaggcttgtttcggaggcatgcaattgggaaccaataagggtggagcaacatacttaccaagttgagtttcctagacgagaggatcttcaacgtttactcactttcggggtaagtaaggttagtggcagcaagtgtttacttgaatttgaggaatgtattaaaccagcaccgcagggtactaggttgcagaaggtttggatccgatttacaggtattccagaaattctgcttaatgacttccttatcacatggagccttggttcgttgattgggaagactgagaaagtagatatgccctttacacgcaagagggggatagccagattgcttgtgatggtgcttgatgttgaccagattcctgattttgctccgtggtcttatgatgatgtgcattatgatcttgaggtggaggtcgagaagatgccacaaaatgagccaaatgatgatgatatcctcatggcagatggggaggatagggataaagatcatgaggatgcgaatgatcaacattccgagaagtcaagagataacaataatcctcatgcctcttctaagaaagagaaactacttattgggggtgcatcttcggcatcaaaagttcccatggagacattacgttttggttcttttgatgtcccaactacaccatataagcttggcagtgagtttgcgaaaaaggcggcgaatgagcaagccccttctgtttcaattcaaagatcggtctccacatctaagctggaagaagtcacgcatttgatcactaaggagaatagaaaaacatcttccgcagtaccgctggctaggaggactgagccactgcatatgcctgagcatgataacacgcagcaagcttgctgtcaaattgctccgtcatcaactactgacagggaggcgcattccattatgagggacagcaagcaagaggcgagtcggccttctaatcctgtttctgcccatatcctctcgaatagtaagcagcaaatgacactttcgaactatgataacattgttgaagggacaaattctgaccagcactttgactctatcctaacagggaatacccacaatgagattactaatgatgatgtgatttcatttggggggatcccggatccttcatcatgtgatagacgtttcagtcaacgtattcaggagaagcctgatgctgatgatatgtcgataggttgtgccatgcgagctgcaaaaattcgagatgctgaaacaacttcaggtttgtctattgatctgactgcatctattttaaaattatctgaatctgaaatcttgaataatgctagtgaattaggaatctctatgggtacatctagtaaaaatatagctaagtccatcaatgatattcttgatctagaagttgacagaaccatagagtttatcaaaaatctagcagcggttaaacctatgaatgattcggatatgagtgaattaggaggtgctggtcttcaaacattatgtgataacatttttcggatagacggggaagtagacgaagtggatgatatgggtttggaggggtatatggccccttcagatgccgacaagcattcacaatctttacatactcataatgatcttaaaagtaatcacgaaataactaggaggccttggaaaaggaaggtgtatcctgaatcagtggttaggaggagtgcgagagttaagcaaaagaaaaaattccatgatgaattatgaaagggattttttggaatagcagaggtctggcggacttggctaaaaaaaggttccttagagaagcatcgatacaatataatctggatttcatggcattacaagagactgggagagataatttcacaacccaattcttaaataatttatcaggaggggttgattttgattggcattgtttaccaccaaggggtagatccggtgggatcttacttggggttaaatgcgaaacgctagaagtgatgagtgtggtctatggtgagttcgccgtcaaatttcgtgtaaggtcgaaagttgatggttttaagtgggctctagtggcagtatatggggcggcacaattggagttcaaacccgattttctagcagatctagtgagaatctgtggagatgaaacattaccaattatggtggggggagactttaatatcattcgaagacagaatgaaaagaataatgacaactttgacacaagatggtcaacgatgttcaatatggttattgagagtttgaatctaagggagattcatctttctggcagacagtacacttgggcaaactctttgccgataccaacgtatgaaaagttggatcgggtcctgactagtgttgactgggagcaaaaatacccattagttacggtacaagcattacagagggcaatctcagatcacgcgcctcttctagttgactcaggggaggccaattatagaggcaacaagaatgtattttctttcgaactaagctggtttgaaagagaagggttcatggatttaattacaaaggaatggggtacacacttaggtggatccacccgtgtcgaaatttggcaaaacaaaattagaaatttgcgccgagttcttagaggatgggcgaaaaaccaaagtggtatttataagaaagaaaaagaaagactaataaacctgattgatgaacttgacgttaaggctgagtcaacgcagctaaattctattgataggaatatcaaaacagatgctgagattagattgcaaaaattactgagagaagaggagatgaaatgggcactaagagctaaggtgatgaacgtggtgcatggggatgataatactaagttctttcgcatgattgcaaatggcaaacatcgaaagaagattattactcagctagagcaagatgagggtactattataggacatgaaaatttaaaggtatatatctcagaatactataggagtctttttggtgcaccagaggagaatatgatctatctggatgaaaccaatgttcaggatattcctcaattaaatgatcaggagaatgaggttctatccgctagattcacagagcaagaggtgtatgacgcaatagtacaaatgaaacacaataaagcatcgggaccagatgggtttcctgcagaattttacaaaaagtgttggcatgttattaagggagatctaatggctatgtttcatgacttattcaatggtcggttagagctgttccaccttaactttggaacaataacgttgttgccaaaaaaggtggacgccatgcgtatccaacaatttagacctatctgtttactaaatgttagttttaagatttttactaagacagccaccaacagattaacaaagatagctgattcagttgttcaaccaacacagtcagcgttcatgcctgggcgacacattttggagggtgttgttgttctgcatgaaacgcttcatgaaatccacacaaagaaactagacggagttatctttaaagtggattttgaaaaggcttatgataaagttaagtggtcttttcttcaacaagccttacatatgaaaggatttagtgaaaagtggagacagcaggttgacacttttatccagaaagggagtgtcggagttaaagttaatgatgacattgctaatttttttcaaacacataaggggttaagacagggggattcaatgtccccaatactctttaatatagtagcagacatgttgacaatacttattgaaagagccaaagagaaaggacaaataggtggtctcattccacatctgatagatgaaggtgtgtccattctacaatatgcggatgatacaatcatttttatggaacatgatatggagaaagcgaggaatatgaaattaattttatgtctttttgaacaactttcgggttaaaaataaattttaacaaaagcgaactcttatgctttgggaaggcaagagaagagcaaaacgcttatcgacagctatttggttgtgagatgggttctctaccttttagctatcttgggattcctattcatcacagacggttaacaattaaagaatggaaatgtattgaagaaagatttgaaaagaaactaagttgctggaagggcaagcttatgtcatacagaggtagattggtcttaataaactctgttttaacaagtctaccaatgtttctcttgtcttttttcgagataccggtaggggtacggaaaagactggatttttatagatctcgtttcttttggcaaaatgatgagaataagaaaaaatacagattagcatgttgggatatgatttgtagacccaaagatcaggggggtttgggaattgaaaatttagaagtaaagaacaaatgtttgctgagcaaatggttatatagaatttccacagagacagagggtatgtggatttaaattttgagaaataagtatctcacatcaagaactctagctcaagctacaattagacccaatgactcacctttctggaaagggttaatgaggataaagagtaatttctttcaaaagggtgaaatttgttgtaggggatggtacattaacaagattttgggaagatacttggcttggggattcaccacttgctttacaatatcccatgctctacaacattgtgcaacgtaagcaggactatgtgtccacagtcttacaatcgatacctataaatatgcacttcagacgagcactagtaggcgcacgttgggaggcatggatgcatctagtaagaaggcttatacaggtacaactatctgatcattcagacagcgttagatggactttaactgtcaatggggttttttcagttaaatccatgtacacacatctggttaattcaggaccattagctagatcgttgcatatttgggatatcaaagtgccgttaaagattaaaatttttatgtggtttttacacaaaggtgtggtattaaccaaagataatttaattaagcgtagttggagaggtagatccaattgttgttattgtgatcaacatgaaactatcagacacttgtttttggactgccccctcgcaaaactactttggcgcactatccatataacttttaacgtgattccacctacgtccatcaattcaatgtttacaacgtggctcaatggagtggacgttaaactatccagacttattagaatagggatttgtgctctcttgtgggctatatggaataccagaaacgacataatatttaatggtaagaaattcaacaattttttgcaggtcatttgccgagcaacgtcctggatccgtacgtggtcatcactcagtcttgcggacaacagggagcttatggctactgggtgcaaccggtgggagatggtcgcacgggttatcttcaaccggtttggatggtgtgctagtaatagactgggtgtataggcatcgtggtctgctattagttacgctggatgtgcgttttttcttaagagtctgtttagtttcagatccattttattttcagccttcaggattgctttgaactattttgttactttggatgtttaataatatggctgcatgcatcgattgatgcagaggctgggggatcgtttcctccttttcaaaaaaaaaaaaatctccaatgaatgatattttcttattatccattttttggtccctgttatttgtcattttctcaaggcactgaattccatgttaatatgactaggacaggattggaccagtacaggatgctatagtaacgcaattggattcttctgttaaccaacaagaggtcaccggtcaagaacatcaccatcacttagatcagccagtagatggtatgcatccaactttagttggcagctagttggcatgtaatgtccattctttggagttctttttttattttggcagccactcacttatttggccattcatctctactcgtctgtgtgtctgccatggcaatgcgcactgctctcaagatcttaactatctcgtctgtgcttgtagttcttgctgcagatcaggtcgaagggcggcatcatttgcctgattgtccttctttctcgtgtggccctctcagaaatgtgtcatctccatttcgtcaggcaagtgatccacctgggtgtggttatccatcttacgagctaatttgcagtggtacgaaggcttcaattcacatcgacgatgcaacatactatgtatcttccatcaactacagtagttcttccttctgggtcgtcgatgccgacatggatttatacaacagctgccctctacctcggtggaatcggcctccgttcagataccagaaccacatggaagtcgaattggaacccctttcatataataaggcttgctttcttacatgctctcgggaagtaaaggacaatggtatgtacatgcctgttgcttgccttagcacaaatcatacttatgtttatgtcttaactggttatggatcttattcaatggagtatctcgagccttcttgtggttacttggctagaattcctcggccttgggacagtccagggccgctagaaaatgcaagtcatgcagatgttgtgaaatccatgaggattggatttgctgttcgattccctttcagatatgacattggcatgagtagaacaaccatcaaagagtgcctaataagccagtttcggtgagttttgcctgtataagtgtcttatcccaaaaccgttaagcccctctttcattccttctatgtctcgttggatggtattgcagcgacttatcgcaggattgtagtgatggcgtcaagagttgtgttcggaaaattcttttggtcgatggagctttctgggagtgctcactgtatgcaactggatatgatgattatccctcagaaatcatagcattagtcttcttctctatgaagtggctagctggtaattccgaatgttctgatcttaattgcagtttcttcatcatcacatctacgcatgtgtgtacccacccactaaaactatataaacttgtttatgcagtgttatgcaggtttgtgttggcaccactggctgtgttgatcttccttgcgcacaagtattggaaaacgaggatcacaattgatgctgtcgagaagttcctccgaatgcaacaaatgctaggtccaatgaggtatgcctacactgacatcacagcaatcacaagccatttcagagataagctgggtcagggaggctacgggtctgtgttcaagggtgtgctattgccaggcaatgttcatgttgctgtcaagatgctagagggcaatcctaactgcaatggagaagatttcatcaatgaagtctccaccattggtaggatccatcacgttaacgtggtgcgcctcatgggattctgctcagaggaaatgaggagagcactggtttacgagtacatgcctcaaggttctctagacaagtacatcttctcatccgggaagagtttttcctgggacaagctcgccaagatagctttgggcattgcaagggggatcgactaccttcatcagggttgtgacatgcagattctgcactttgacatcaagccacacaacatccttctcgacaacaattttgtccccaaagttgctgattttggtcttgcgaaactatacccaagggacaacagctttgtgccatggagagccctacggggaacaattgggtacatagctcctaagatgatatctcgtagcttcggcgccatatcgagcaagtctgatgtttacagctttgggatgctgctgctagagatggccggaggaagaaggaatgctgatccaaatgcggcaaacacgagccaagcgtactatccgtcgtgggtgtatgaccggctaactcaacaagatcatgtgggtgagatatttgctcatgtttatactgagatgcatgagttggagaggaagttgtgcattgttggactatggtgcattcaaatgaggtgtcaagaccgaccgacaatgaatgatgtcatagagatgctggaaggtggcatccatagcttgcaaatgccttcgaggccattcttctgtgatgaagggcacatccatgccgaggatacttaccatttgtcgtccgagctgactgaaatctccgaggacatgagtgcttagctcataaaaatattgaaatgatggttgtatagttgtatcgagtatgtaattataggtggaaacatgtaatagctagcagagcagcagaacctttgtgctgctttgtgttttcttttattgcccaaacctttgtgctgggactccagttttccttggatgtatttcatgatgaagctggttggtgtcgcctaaaaagtcctcaagctaagtgctatttttgtatcaacgtatattttgatatttcttttaccgaatacacgcttgtctaaatccgatagaattgtcagttacaagttgggaagatatggagagcctcaagtgccggatcttttaagcaacgagttatttgcgtaataaaactgagactatgtaattttctaatcctcctttttaactttaaagtgttagagtatatataatatagtcatgtacccttttgtatttatcccattgtataaggggttctctgcatatagtccacatctgtacatgtatatatatcgacctatggcctcatgggaatacaagttgcatattcctaacatggtattagagttaggtcaattttttgcacgctgcaactcgtgcgattgatccgtctcctcaccccgatcgaagcccgttctcgatctcctcctcctgccccgtttcgctcgcaacctctcctctcccgctcgaagctgctcgtcctgtccgatttgctcgtcggcctgctgcgtcccgcccgatctgctcgacctgcgtcccgcccgatctactcgacccgctcccgatctgctcgacccgctcagcctccagatcgagcc

SEQ ID NO: 22 - protein, 657 aa, Hordeum vulgare
MAMRTALKILTISSVLVVLAADQVEGRHHLPDCPSFSCGPLRNVSSPFRQASDPPGCGYPSYELICSGTKASIHIDDATYYVSSINYSSSSFWVVDADMDLYNSCPLPRWNRPPFRYQNHMEVELEPLSYNKACFLTCSREVKDNGMYMPVACLSTNHTYVYVLTGYGSYSMEYLEPSCGYLARIPRPWDSPGPLENASHADVVKSMRIGFAVRFPFRYDIGMSRTTIKECLISQSDLSQDCSDGVKSCVRKILLVDGAFWECSLYATGYDDYPSEIIALVFFSMKWLALCRFVLAPLAVLIFLAHKYWKTRITIDAVEKFLRMQQMLGPMRYAYTDITAITSHFRDKLGQGGYGSVFKGVLLPGNVHVAVKMLEGNPNCNGEDFINEVSTIGRIHHVNVVRLMGFCSEEMRRALVYEYMPQGSLDKYIFSSGKSFSWDKLAKIALGIARGIDYLHQGCDMQILHFDIKPHNILLDNNFVPKVADFGLAKLYPRDNSFVPWRALRGTIGYIAPKMISRSFGAISSKSDVYSFGMLLLEMAGGRRNADPNAANTSQAYYPSWVYDRLTQQDHVGEIFAHVYTEMHELERKLCIVGLWCIQMRCQDRPTMNDVIEMLEGGIHSLQMPSRPFFCDEGHIHAEDTYHLSSELTEISEDMSA

SEQ ID NO: 23 - DNA, 1038 bp, Hordeum vulgare
atggtggccattgtgatatatttctccttgaagtcaaggtacaatgaagagataaatatgaaggttgaaatgtttctcaaggcatatggcacatcgaaacctacaagatacaccttccgtgatgttaagaagatagcaagaaggttcaaggataaactcggtcagggtggattcggaagtgtgtacaaaggcgagctaccaaatggagtacccgtggcagtcaagatgctagagagctctacaggagagggggaggacttcatcaatgaagttgcaaccattggactgatccaccatgcaaatatcgtacgtctcttgggcttttgctccgaaggaatgagacgggcccttatttatgaattcatgcctaacgagtcactggagaaatacatattcccacaagttccgaatatctctcgacagctcctagcacccaacaaaatgctagatattgctttaggcattgccagaggaatggaatacctgcatcaaggctgcaaccagcgcatcctccactttgacatcaagccacataacatcctgctggactacaacttcaatccaaagatctcagactttggccttgcaaagctgtgtgcaagggaccaaagcatcattaccttgactgcagccagaggcacaatgggatacatcgcaccagaggtatattctcggaactttggaggggtgtcttacaagtcagacgtgtacagtttcggcatgctggtgctagaaatggtgagtggaaggaggaactcagacccaagtgttgagtaccaggacgaggtatatctccctgaatgggtctacgagagagtaatcagtggacatgaatgggagcttacttcagaaatgacaggagaagaaaaagaaaagatgaggcagctgactattgtggccctgtggtgtatccagtggaacccgaagaatcggccatcaatgacaaaggtggtaaacatgttaacggggaggttgcagaacctacagattccccctaagccctttgtgtcgtctgaaaatcatcatcctagaacataa

SEQ ID NO: 24 - DNA, 8490 bp, Hordeum vulgare
tacatccatggatggctcatctctgcgttatacgacagtatatcaaatacaatcacgatcctaaaaattagtaccgcacggtctaaacgaggaaatatagttgctaaccttttagaataagtagaaataaagaggaagaggtttaagcatggctcgggcatctcatatcgtagttgtgttcggtgaactgaagcggcatcgctctaacacacatttcaacaaacacctctagtgcatcaaaaaaagtggagagcaagcacccacccataatcctccatccaagaaaaatgcaacgaagaggggagaggggggctgtgctatatataggcagatgactttagtcccggtttgagacacaaaccgggactaaaggtgacgcacatgcgggctgcccaccgcgtagccctttagtcccggtttgggacacgaaccgggactaaaggctccttacgggctgggactaaagcctcgagggaggaaatgagaattgaggcgatgtggccgggcctttagtcccggcccagaggcaggccgggactaaagggtccaggccaaaggcccgttttctactagtgaggataggcttatcaaacttatagaggatcttgatcgtaaagctgagacaattgttctcgatttcaacgagcgatatttgaagaatgaagctgaacagcgcctttgagtgctccttagaggggaggaatcgaaatgggctactagagctaaagtgcgggcaatcgtttatggggataataatactaaattttttcacctgattgcaaatggcaagcataggaaaaagagaattctcgagcttgagcaagatgagggtacaatagtaggtcatgagaacctaaaattgtatatttcaaactattataaaaagttattcggtgcttctgaccctagttttgtgactatggatgaacatcaaattgcggatatccctcaagtcggagaggctgacaatgagttactttccgccccatttacggagaaagaggtgttgaaggcaattggggacatgaagaatggtaaagctcccgggccggatgggtttccagctgagttttacaagaaatgttggcacattatcaagaaggatcttatggcaatgtttcatgatctttacgatggtcacttacaactctttcatttgaattttggtaccattacgttgttaccaaagaaagagggagcaactcgtattgagcaatttctccctatttctttgctaaatgttagtttcaagattttcacgaaggtgggaacggatagactaacgactatggcacattcggtggtgcaatctacgcaagcatcatttatgcctgggcggaatatcctagaaggtgtagttgtgctgcatgaaacgttgcatgaaattcattcgaagaaactggacggtgttatcttcaaagtggatttcgaaaaggcatatgataaagttaaatggtcttttctgcagcaaaccctgcgcatgaaggggttcgacgagctttggagaaagcatgtcgatttttttataaaaaagggaagtgtcgggattaaggtaaatgatgatgtaggtcatttgtttcagacactgaagggtcttagacaaggggacccaatgtctcccattttattcaacatcgttgctgatgtgttggcacttatggttaagcgtgccaccgagaaggatctcatagggggtctcatcctgcatctcgtggacgggggtgtttctatattgcaatatgcagatgacacaattctgtttatggagcacgatctggttaaagctagaaatttgaagctcatcctttgcctttttgcgcagctatcagggctgaaaattaattttaataaaagtgaggttttctgttttggcagagccaaaaatgaacaacacacttataatcaattgttcggttgcgaaattgggaatttgcccttcacgtatttagggattcctatacatcaccgaaaattatcaaaaaaagaatggaaatgcaatgaggatcgatttgaaaagaagctaagttgctggaaaggcaagttgctatcttatggaggccggttagttctggttaattcggtattaactagcatgcatatgtttctcctatccttcttcgaagttcccgttggggtgtgaaaaagattggatttttatcgggcccgttttttctggcaaagcgatgaacttaagactaagtacaggcttactagatgggatattatttgtagacaaaaagatcaagggggattaggaattgaaaatttagagaccaaaaacagatgtctactaagcagatggctgtttagactatcttcgaaaaatgaaggtatgtgggtacaaattctgaagaacaagtacttacacaacaaaaccttatctcaagttacagcacgaccaactgactctcctttctgaaaggggttgatgcggacaaaaaatgctttcttcagcagaactaggtttatcataggcaatggtgaatctactagtttctgggaagacacttggttaggggagatgcctttagctattcaatacccgcggctgtaccatattgcacaacatcgacaagcgtccgttgcgtcagtacttggtgagattcctctaaatattcaattccgtagatctctcatcggcgatagatggattagttggttgcacctcgtgagaaggttgatggacgtcaacctctctcacagacccggtcgtgtttattggaagttgaccacgaatggagtattcacggtaaaatctatgtacatgcatctcatagattcaacacctattccaaaatttgcgcatatttggaagataaaagttcctcttagaatcaaagtctttatgtggtttgttgacaaaggagttattctaacaaaggataatcttaccaagcgaaactagaatggtaatactagatgcagcttttgtccgaataaagaaagtattaagcacctctttctcgattgtcctatggctaaactggtttggcggtcgctgcacattgcttttgacatcactccccctaatagtataaacacgttatttgggatgtggttacagggggttgatgtaaacactgctaaacttattcgctgggttcttgtgcactgttgtgggctgtatggaacaacagaaatgacttagtatttaacagacaacgcgactttaacttcttgcaggttatcttcagggctaccgcactgatccgcacatggtcattactcactcctgtggaagccaggaagcctatggttactgggtgtgtccgctgggagatggtagcacagggtatcttcaaccggtttggatggcgtcatgttaataggataggtgtttagtcatctatatattttgtcatctccggcttgggcaagaaggatgccttgtatcgtcctattttattttttgttgcacacttcatgtattttgtctgagacttcatttcgattttaatatatttggccgtatgcatctctcgatgcagaggccggggctcagcctccatttcgaagaaaaaatgtactcttctcctctcctcttgttttttgttgattgacttgaatatccagtcaactccttcttttttatggccaacttgtaaatagcctggtcttggcagtatcaagatggcgtatcaaggagatgtagcaatggaaaatggcctatcaggacttaaatggtagtacaaagttgagtcattttttggtcacttattttgaaacggagggagtagaatgttataggtagggaccaagaacaaaccattggtcgtccaggccaaaatgatcttgatgcacatgttttttatgctttatagcatgtcaacttcaagaatttgttttttactacatgataattatacaagctccatgtacttagaggtttggaatcctattggagtactagaggatatatgtgtggtgaaagggcatgaatggtctaaacagtaatagaaccacgggactttctgagccatacaacacgtatgacagaggttttggtggcagctggaattcctggccaggcgcattactttcatgacaagcgccaccactatcctcaccagaggttaaagcaaaaagccatttttaatactcctgcctttctcttcttccatcttcatacccaatacatcccatggactttcttaaatttcttgtcacgctgctgctgctatctcttctcacctaccaaacctatgtaagtgcagcatcggatgatgaagatttctcaaaacaatgtccatctcacaggtgcagcaaacatggacctgagatccggttcccgttcgggctgtcaactcacatcatcatgcggcgcaccaggtatgcagttatcgtgctccggggatgacacaatcctggatcacccggttcttggctcctgcaaagtaaccgagatctattacagacactgtgtcatcaacctcaccccgcttgtggaaccatcgacacagtgcccacttcagaagctcatctcaacaaatttagcaactggtgtgtacaaactacctccatcacaaacgaccctggtacgttgttcaagtgaattcatacctgcagatccatacagtgtagctggcccagctgcttgtctcagtaacaacgcaacccaattctggtatttggcggagtcagctgatgcatacatatatgatctaccacgagactgtgaggctgtttctagaggcattccagtaccctacagctatgatatagatggcccaaacttacgagacgatcttgccttcaacaaaaaagcgagtgcagtcatcagttttggtgaaacaacattcacttggtacctcagtaaccttacctatgtctgccaacagtgtgaacgtgaaggtcgacactgtggattcagttcacgaagccgtcaagcattctgccagccccatggtattctttctttcccttggtgtgtcaatttattttggaacaacaattcaatctcttatattccggtgttaactgttttaaatcttccaataggattttcacctgtagttcaattatttaacaaacaaaattgtaatgcaaatcatagaaattgttaagttgttcaagcacatacagtctctaggttcacatccttgaaagttcccaccaaaaaatatccgttaagagatgcaagtgccaaattatatgggaaaacctatggtggtactaatctgtaaacttctcggcgcataaaaagtaagagataaaccttagttgacaattcttcatatcggtttagtatttgatatctgaatatgaataaataagcgtacaactataacataacaaagaataattaatcgatattacaccatatgtctaaggtccaatagacatgaagccaaccggaaggtagcaggcacacacgcacgcacgcacgaatcaggaattagttgttccaaacatactttgtagaagtaatgaagtagtcttctgttttcatctacattcaaattaaatcatggaaaaaactaatgatattcttcctacatgcaaatgcaggtacccatgtcgtcccaattgcgggtaatgttgcaactcttttccttttgctcattctaaatgaaattcttgttgcttaccactgtaactcctttgtgttttcccattacaataagagtcgggaatgcatggcctcttatctgaatgcaattagctaatttcctttattttttctaatcagcagcctcatctgtagctgcatttgtagttttttcatcgatggtggccattgtgatatatttctccttgaagtcaaggtacaatgaagagataaatatgaaggttgaaatgtttctcaaggcatatggcacatcgaaacctacaagatacaccttccgtgatgttaagaagatagcaagaaggttcaaggataaactcggtcagggtggattcggaagtgtgtacaaaggcgagctaccaaatggagtacccgtggcagtcaagatgctagagagctctacaggagagggggaggacttcatcaatgaagttgcaaccattggactgatccaccatgcaaatatcgtacgtctcttgggcttttgctccgaaggaatgagacgggcccttatttatgaattcatgcctaacgagtcactggagaaatacatattcccacaagttccgaatatctctcgacagctcctagcacccaacaaaatgctagatattgctttaggcattgccagaggaatggaatacctgcatcaaggctgcaaccagcgcatcctccactttgacatcaagccacataacatcctgctggactacaacttcaatccaaagatctcagactttggccttgcaaagctgtgtgcaagggaccaaagcatcattaccttgactgcagccagaggcacaatgggatacatcgcaccagaggtatattctcggaactttggaggggtgtcttacaagtcagacgtgtacagtttcggcatgctggtgctagaaatggtgagtggaaggaggaactcagacccaagtgttgagtaccaggacgaggtatatctccctgaatgggtctacgagagagtaatcagtggacatgaatgggagcttacttcagaaatgacaggagaagaaaaagaaaagatgaggcagctgactattgtggccctgtggtgtatccagtggaacccgaagaatcggccatcaatgacaaaggtggtaaacatgttaacggggaggttgcagaacctacagattccccctaagccctttgtgtcgtctgaaaatcatcatcctagaacataaaagcacagagaatgctgccattatttgaagacatgcacttagtccatgtgttgcacaacaatacatagcactagcgtgcattatatttccttctttccactctattacttattagcacctgattgtatgaataatcgtccgtccccatgtaataaaatactactagcaggtatatgcaagaccatatgagaactttatttggactgtataataacagttagaaatgcagtggtttgttgcttaccttgttgtgatgctccgctgatttatactatttcctacagtgcattattccacactgggatcctcaggaaacatgattgtcatttccattgtgctgagaacatcacagaatatctatttccctgttccttcaaatatcagcagtcgactgaacatgttccgatctttcccctcccctgtgctagcacctgccagctgatgcatgaataaccagtcatcaccatgtaatggtactagtagatatttgcagcatatcggaatcacatggagctttgttctgcggatcgacgcttctgtaactaagctctttctaatgcgcagatcctgaaagtaatcgtgtcactaaaaacgaagtgagagcaagggggattactcaccggagcagaggaatgggggcgcttcgccggagcccaaggaaagacgaggagctcgcttagacgccggagaattagggcgggaatcgctcgccggcagccacaagggtccgagttgcaaaaaatgtggatacgtctttctgtccggaaaatattttttaaacacaatattcgtcactgtccgccacaagggttataaacgggcagagttgggctcccctgcaaaaggggatttttattcgtcactgtctttactttgcaattgaatttttttaacacaatacgatcgaagtcgcttgcatacatgagcataacttgtcgttatgcatgcacacacgcacgcacgcatactctacttctttgagcatctccgagagactgagtcgacaaagcatcttcagactttacaaagtcatcacatgtgtctcgcctcgacgggatgtctcctttcactgaacgaacactgacagaagccaaaaataaattcaaaaataatgcgagcaccaacttcaagcctagaacttgaactctcacgggacaggaatatttctgttctcctaaccatccaaccaccaattatttaaaaacgctttcatcgatcaacaacatcagtcgcacgttctgttgtttgatcggcatcgatcacatggtgtggcatgcagcaggtgtgataattcctaagtaccaagaattgtcgtagcactttttaattatgcagccacttgagaggtcatctatcgtcgaggaccaaaacgtgttgaggtatggggagaggcaagggggacgagccgtgtaaaacgggcgggccttgtttgcacgcacgaaccattgagttgttttcaccataagaatgatgtatcgataagaactagggttgggcattgtggtagacttgcacacgcaataatacaaactgatcaactgtttttgggtgtttcttatttcccatgaaggtatctggatatcaaccaactaaccataagtgtctctctagtagatcttgatatattgtgtcggcttagtctttcaaagatgcttataagaataacatgtgcgttcgttaaggtgagtgtatacactagtagaaaacgggcctttggcctggacc

SEQ ID NO: 25 - protein, 345 aa, Hordeum vulgare
MVAIVIYFSLKSRYNEEINMKVEMFLKAYGTSKPTRYTFRDVKKIARRFKDKLGQGGFGSVYKGELPNGVPVAVKMLESSTGEGEDFINEVATIGLIHHANIVRLLGFCSEGMRRALIYEFMPNESLEKYIFPQVPNISRQLLAPNKMLDIALGIARGMEYLHQGCNQRILHFDIKPHNILLDYNFNPKISDFGLAKLCARDQSIITLTAARGTMGYIAPEVYSRNFGGVSYKSDVYSFGMLVLEMVSGRRNSDPSVEYQDEVYLPEWVYERVISGHEWELTSEMTGEEKEKMRQLTIVALWCIQWNPKNRPSMTKVVNMLTGRLQNLQIPPKPFVSSENHHPRT

SEQ ID NO: 26 - DNA, 33 bp, artificial
  forward primer
tacctacccgggatgcccactgcccttaaggcc

SEQ ID NO: 27 - DNA, 39 bp, artificial
  reverse primer
tacctagcggccgcctactcatcatcctcctccgagatc

SEQ ID NO: 28 - DNA, 35 bp, artificial
  forward primer
tacctacccgggatgggggacgggaagaggtactg

SEQ ID NO: 29 - DNA, 37 bp, artificial
  reverse primer
tacctagcggccgctcactttgcgttggcgagcaacc